SEQUENCE LISTING (3 sequences)

SEQ ID NO: 1 - DNA, 39 bp, synthetic construct
  source 1..39: mol_type = other DNA
  modified_base 1: mod_base = OTHER; note = 5' Azide thymidine
  modified_base 37: mod_base = OTHER; note = Adenosine phosphorothioate
  modified_base 38: mod_base = OTHER; note = Guanosine phosphorothioate
ttttttttttttttttagacaccgttccttggacagagc

SEQ ID NO: 2 - DNA, 80 bp, synthetic construct
  source 1..80: mol_type = other DNA
  modified_base 1: mod_base = OTHER; note = 5'-Phosphate guanosine
gaacggtgtctattatgtcctatcctcagctattatgtcctatcctcagctattatgtcctatcctcagctctgtccaag

SEQ ID NO: 3 - DNA, 21 bp, synthetic construct
  source 1..21: mol_type = other DNA
  modified_base 1: mod_base = OTHER; note = 5' ATTO 647N or ATTO 565 thymidine
  modified_base 21: mod_base = OTHER; note = Inverted deoxythymidine
tattatgtcctatcctcagct

(19) United States
(12) Patent Application Publication
(10) Pub. No.: US 2024/0264154 A1
(43) Pub. Date: Aug. 8, 2024

(54) SINGLE MOLECULE ASSAYS FOR ULTRASENSITIVE DETECTION OF ANALYTES

(71) Applicants: President and Fellows of Harvard College, Cambridge, MA (US); The Brigham and Women's Hospital, Inc., Boston, MA (US); Massachusetts Institute of Technology, Cambridge, MA (US)

(72) Inventors: David R. Walt, Boston, MA (US); Connie Wu, Boston, MA (US); Richard Novak, Cambridge, MA (US); Tyler Dougan, Boston, MA (US); Rushdy Ahmad, Cambridge, MA (US)

(73) Assignees: President and Fellows of Harvard College, Cambridge, MA (US); The Brigham and Women's Hospital, Inc., Boston, MA (US); Massachusetts Institute of Technology, Cambridge, MA (US)

(21) Appl. No.: 18/618,019

(22) Filed: Mar. 27, 2024

Related U.S. Application Data
(63) Continuation of application No. PCT/US2022/045798, filed on Oct. 5, 2022.
(60) Provisional application No. 63/341,540, filed on May 13, 2022.
(60) Provisional application No. 63/252,440, filed on Oct. 5, 2021.

(51) Int. Cl. G01N 33/543; C12Q 1/6844; G01N 15/14

(52) U.S. Cl. CPC G01N 33/54326; C12Q 1/6844; G01N 15/14

(57) ABSTRACT

The invention provides ultrasensitive methods for detection and quantification of target analytes in samples. The methods can be multiplexed to allow simultaneous detection and quantification of multiple target analytes. The methods can achieve an attomolar limit of detection. The invention also provides related compositions and kits.

### RELATED APPLICATIONS

This application is a 35 § U.S.C. 111(a) continuation application which claims the benefit of priority to International Application No. PCT/US2022/045798, filed on Oct. 5, 2022, which, in turn, claims the benefit of priority to U.S. Provisional Application No. 63/252,440, filed on Oct. 5, 2021, and U.S. Provisional Application No. 63/341,540, filed on May 13, 2022. The entire contents of each of the foregoing applications are incorporated herein by reference.

### GOVERNMENT SUPPORT

This invention was made with government support under EB029777 awarded by the National Institutes of Health. The government has certain rights in the invention.

### SEQUENCE LISTING

The instant application contains a sequence listing which has been submitted electronically in XML format and is hereby incorporated by reference in its entirety. Said XML copy, created Mar. 15, 2024, is named 117823_32503_SL.xml and is 7,097 bytes in size.

### BACKGROUND OF THE INVENTION

The ability to accurately measure extremely low levels of molecules, such as proteins, nucleic acids, and metabolites, is essential for a wide range of clinical and environmental applications, including disease diagnostics, drug discovery, pathogen detection in food, environmental toxin detection, and bioprocess control. Ultrasensitive measurement techniques are especially critical in clinical diagnostics, as many potential biomarkers exist in accessible biofluids at levels well below the detection limits of current laboratory methods (Cohen, L.; Walt, D. R., Chemical Reviews 2019, 119 (1), 293-321). Digital measurement methods, such as digital enzyme-linked immunosorbent assay (ELISA), have vastly improved measurement sensitivities by up to 1000-fold over traditionally used analytical techniques such as conventional ELISA (Rissin, D. M. et al., Nature Biotechnology 2010, 28, 595; Rissin, D. M.; Walt, D. R., Journal of the American Chemical Society 2006, 128 (19), 6286-6287; Rissin, D. M.; Walt, D. R., Nano Letters 2006, 6 (3), 520-523; Yelleswarapu, V. et al., Proceedings of the National Academy of Sciences 2019, 116 (10), 4489).

However, the sensitivities of digital measurement techniques remain insufficient for many diagnostic applications, particularly for measuring disease-related proteins. For instance, while several protein biomarkers for neurological disorders have been shown to be upregulated in cerebrospinal fluid, highly invasive lumbar punctures are required for these measurements, thus making it impractical to screen individuals for early disease detection (Robey, T. T.; Panegyres, P. K., Future Neurology 2019, 14 (1), FNL6; Olsson, B. et al., The Lancet Neurology 2016, 15 (7), 673-684; Galasko, D. R.; Shaw, L. M., Nature Reviews Neurology 2017, 13 (3), 131-132; Fortea, J. et al., The Lancet Neurology 2018, 17 (10), 860-869). As only a small fraction of brain-derived proteins passes through the blood-brain barrier into circulation, highly sensitive techniques that can detect and identify rare protein biomarkers through a simple blood test are crucial for addressing this unmet diagnostic need (Hampel, H. et al., Nature Reviews Neurology 2018, 14 (11), 639-652; Simrdn, J. et al., Current Opinion in Neurobiology 2020, 61, 29-39; Parnetti, L et al., The Lancet Neurology 2019, 18 (6), 573-586). Improving analytical sensitivity is also a major challenge in other diseases for which rapid point-of-care (POC) diagnosis is essential for effective medical intervention but where easily accessible biofluids, such as saliva or urine, are required. These biofluids contain only a minimal serumnal component, necessitating ultrasensitive techniques for protein biomarker detection.

One main barrier towards increasing sensitivity in digital ELISA is low sampling efficiency. While digital ELISA methods utilize single molecule counting to improve measurement sensitivity, low sampling efficiencies limit the number of target molecules that are counted. At very low target concentrations, the Poisson noise from counting single events, √N, where N is the number of counted molecules, contributes significantly to measurement error. As an example, at a sampling efficiency of 5%, only 30 out of the 600 target molecules in 100 μL of a 10 aM sample will be counted, assuming perfect capture efficiencies. The theoretical Poisson noise-associated coefficient of variation (CV), 1/√N, is 18% at this low sampling efficiency and in reality much higher when accounting for capture efficiencies well below 100% and experimental error. This high measurement uncertainty therefore poses a major limitation for detecting rare molecules. Increasing sampling efficiencies to count more target molecules can thus greatly improve measurement precision and sensitivity but remains a challenge in digital ELISA.

Existing digital ELISA approaches utilize microwells or water-in-oil droplets to isolate individual beads carrying single target protein molecules (Rissin, D. M. et al. Nature Biotechnology 2010, 28, 595; Yelleswarapu, V. et al. Proceedings of the National Academy of Sciences 2019, 116 (10), 4489; Kim, S. H. et al., Lab on a Chip 2012, 12 (23), 4986-4991; Witters, D. et al. Lab on a Chip 2013, 13 (11), 2047-2054). The current state of the art for digital ELISA is Single Molecule Arrays (Simoa), which captures single target molecules on antibody-coated paramagnetic beads and isolates individual beads into femtoliter-sized microwells for single molecule counting (Rissin, D. M. et al. Nature Biotechnology 2010, 28, 595). A large excess number of beads over the number of target molecules in the sample is used to ensure digital measurements, where each bead has either zero or one captured target molecule and follows the Poisson distribution. Each captured molecule is labeled with a biotinylated detector antibody to form an immunocomplex sandwich, which is then labeled with the enzyme conjugate streptavidin-β-galactosidase (SOG). The beads are subsequently loaded, along with fluorogenic enzyme substrate into the microwells, each of which can fit at most one bead. Upon sealing of the microwells with oil, a high local concentration of fluorescent product is catalytically generated in each well that contains a bead carrying an SpG molecule. Thus, the number of target molecules is measured by counting “on” and “off” wells.

While Simoa can achieve sub-femtomolar limits of detection and is the current gold standard for ultrasensitive protein detection, its sensitivity is limited by low sampling efficiencies. Only about 5% of the total number of beads can be loaded by gravity (or magnetic attraction in the case of the Quanterix HD-X Analyzer) into the microwells and analyzed (Wilson, D. H. et al., Journal of Laboratory Automation 2015, 21 (4), 533-547). Other methods to improve bead loading have also been explored, including electric field-directed bead loading, hydrophilic-in-hydrophobic microwell arrays, and digital microfluidics (Barbee, K. D. et al., Lab on a Chip 2009, 9 (22), 3268-3274; Decrop, D. et al., ACS Applied Materials & Interfaces 2017, 9 (12), 10418-10426; Decrop, D. et al., Analytical Chemistry 2016, 88 (17), 8596-8603). While these methods have increased bead loading efficiencies, demonstrations of their improvements in digital immunoassay sensitivities remain limited. Magnetic-meniscus sweeping, which uses a combination of hydrodynamic and magnetic forces to increase bead loading, has improved sensitivity, but as in the other approaches, complex fabrication methods and workflows limit its use in POC applications (Kan, C. W. et al. Lab on a Chip 2020, 20 (12), 2122). Another strategy for improving sampling efficiency in digital bioassays is bead encapsulation in water-in-oil droplets. Digital droplet-based immunoassays have been demonstrated with up to 60% bead loading efficiencies and have shown equal or improved sensitivities of up to an order of magnitude higher than that of the current Simoa technology (Yelleswarapu, V. et al. Proceedings of the National Academy of Sciences 2019, 116 (10), 4489). While droplet microfluidic systems are well established for diverse applications, the need for highly controlled, high-throughput droplet generation introduces additional fabrication and processing steps that introduce more complexity when integrating into POC systems. Furthermore, as a significant fraction of droplets do not contain beads but must still be imaged, improving imaging throughput remains another challenge towards POC implementation.

Thus, there remains a need in the art for high-sensitivity and quantitative detection approaches that can be used to detect and measure the concentration of analytes, with high sampling efficiencies and simple fabrication methods and workflows for point-of-care (POC) applications.

### SUMMARY OF THE INVENTION

Measurements of low levels of analytes, such as proteins and nucleic acids, are important for clinical diagnostic applications, but remain challenging due to insufficient sensitivity. Ultrasensitive digital ELISA techniques, such as Single Molecule Arrays (Simoa), have been developed to achieve detection of very low concentrations of disease-related proteins, but still face limitations such as large and complex instrumentation, high cost, and complex workflows, making point-of-care applications impractical. Furthermore, there are still many potential disease biomarkers that exist below the detection limits of current digital detection methods.

Described herein are ultrasensitive detection methods that address the above-mentioned challenges. The vastly simplified readout process and improved cost-effectiveness of the present methods can facilitate potential integration into a POC system. In particular, the present invention provides a streamlined digital ELISA technology, Molecular On-bead Signal Amplification for Individual Counting (MOSAIC), that achieves attomolar sensitivities and requires only common laboratory infrastructure, thus vastly increasing the accessibility of ultrasensitive protein detection. The invention utilizes on-bead signal generation and, thus, does not require bead isolation into individual containers such as microwells or droplets for signal compartmentalization, and eliminates the requirement for complex microfabrication or droplet generation.

Furthermore, the present invention focuses on the use of flow cytometry as a detection approach, and flow cytometry is readily accessible by many research labs and can also be adapted to microfluidic systems at low costs, making the invention more amenable to rapid incorporation into existing laboratory infrastructures and point-of-care formats (Asghari, M. et al. Scientific Reports 2017, 7 (1) 12342). By integrating methods for localized signal amplification of single molecules with the rapid, high-throughput detection capabilities of flow cytometry, low attomolar limits of detection were obtained with an order of magnitude improvement over the gold standard digital ELISA method, Simoa. While other digital measurement strategies with flow cytometric readouts have previously been developed, these methods have been limited to femtomolar limits of detection or above, with attomolar sensitivities yet to be demonstrated for protein detection. A key advantage of MOSAIC over previous digital ELISA methods is the much faster, automated signal readout-less than one minute per sample. Automation via the 96-well plate sampling modes that are already built into many benchtop flow cytometers further provides a streamlined workflow.

In addition to applying a rapid on-bead signal amplification strategy, the high sampling efficiency of MOSAIC can be exploited to systematically reduce assay bead numbers for enhancing sensitivity to detect low attomolar protein concentrations. The improved sensitivity of MOSAIC enables the measurements of previously undetectable analytes in biological fluids, such as low-abundance cytokines in saliva. Furthermore, MOSAIC expands the multiplexing capabilities of digital ELISA, as the number of bead types that can be analyzed within a single sample is no longer limited by the total number of microwells or other compartments. As an example of increased multiplexing in MOSAIC, eight protein targets have been simultaneously measured with attomolar to low femtomolar sensitivities using low volumes of plasma.

The enhanced sensitivity, simplicity, and versatility of MOSAIC compared to existing digital ELISA methods provide a considerable advance towards making ultrasensitive protein detection widely accessible and discovering previously undetectable biomarkers for diverse clinical applications.

Accordingly, the present invention provides, in one aspect, a method of detecting a target analyte in a sample, the method comprising: (a) contacting a sample containing or suspected of containing the target analyte with a plurality of beads comprising a capture moiety that specifically binds to the target analyte, under conditions and for a time sufficient for the target analyte in the sample to bind to the capture moiety, wherein a plurality of the beads are associated with zero target analyte molecule; wherein a plurality of the beads are associated with one target analyte molecule; and wherein at least about 20% of the beads are associated with either zero or one target analyte molecule; (b) contacting the product of step (a) with a detecting moiety that binds to the target analyte, (c) contacting the product of step (b) with a signal amplification moiety that binds to the detecting moiety to generate a detectable signal for each bead carrying the target analyte; and (d) detecting the detectable signal by flow cytometry, thereby detecting the target analyte in the sample.

In some embodiments, the bead comprises a magnetic bead, a paramagnetic bead, a non-magnetic bead, a porous bead, or a glass bead.

In some embodiments, the capture moiety comprises an antibody, an aptamer, an antibody mimetic, a polypeptide, a nucleic acid, a molecularly-imprinted polymer, a receptor, a binding protein, or a small molecule.

In some embodiments, the detecting moiety comprises an antibody, an aptamer, an antibody mimetic, a polypeptide, a nucleic acid, a molecularly-imprinted polymer, a receptor, a binding protein, or a small molecule.

In some embodiments, the signal amplification moiety comprises an enzyme and/or a nucleic acid molecule.

In some embodiments, the detectable signal is generated by rolling circle amplification followed by hybridization with a complementary fluorescently labeled DNA probe; rolling circle transcription; hybridization chain reaction; loop-mediated isothermal amplification; radical polymerization; tyramide signal amplification (TSA); enzyme-catalyzed proximity labeling (PL) polymerization; labeling with a pre-amplified signal with fluorescently labeled enzymes, nanoparticles or nucleic acid concatemers; polymerization-based signal amplification; or magnetic bead-quantum dot immunoassays.

In some embodiments, at least about 20%, 30%, 40%, 50%, 60%, 70%, 80%, 90%, 95%, 98%, or 99% of the beads are associated with either zero or one target analyte molecule.

In some embodiments, the detecting moiety and the signal amplification moiety are linked directly.

In some embodiments, the detecting moiety and the signal amplification moiety are linked by a non-covalent affinity binding pair, wherein the detecting moiety is linked to a first member of the non-covalent affinity binding pair, and the signal amplification moiety is linked to a second member of the non-covalent affinity binding pair.

In some embodiments, the non-covalent affinity binding pair is biotin-streptavidin, biotin-avidin, ligand-receptor, antigen-antibody, or antibody binding protein-antibody.

In some embodiments, the beads comprising the capture moiety for the target analyte are different from beads comprising a capture moiety for a non-target analyte, optionally, with different colors, shapes, or sizes.

In some embodiments, the method further comprises detecting the beads comprising the capture moiety by flow cytometry.

In some embodiments, the method reduces cross-reactivity or non-specific binding. In some embodiments, the cross-reactivity or non-specific binding is reduced by detecting the beads and the detectable signal by flow cytometry.

In some embodiments, the target analyte is a protein, a nucleic acid, a polysaccharide, a lipid, a cell, a fatty acid, a therapeutic agent, an organism, a virus, a toxin, a peptide, an oligosaccharide, a lipoprotein, a glycoprotein, a glycan, or a hormone.

In some embodiments, the sample comprises a biological sample, an environmental sample, or a synthetic substance.

In some embodiments, the biological sample is (i) a body fluid selected from the group consisting of lymph, whole blood, plasma, serum, a blood fraction containing peripheral blood mononuclear cells, urine, saliva, semen, sweat, lacrimal fluid, synovial fluid, cerebrospinal fluid, feces, mucous, vaginal fluid, and spinal fluid, or (ii) a breast tissue, a liver tissue, a pancreatic tissue, a cervix tissue, a lung tissue, a renal tissue, a colonic tissue, a brain tissue, a muscle tissue, a synovial tissue, skin, a hair follicle, bone marrow, a tumor tissue, a tissue lysate or homogenate, or an organ lysate or homogenate.

In some embodiments, the biological sample is plasma.

In some embodiments, the biological sample is saliva.

In some embodiments, the method further comprises measuring a concentration of the target analyte in the sample, wherein the concentration of the target analyte in the sample is related to the level of the detectable signal.

In some embodiments, the method further comprises detecting or measuring a concentration of an additional target analyte in the sample.

In some embodiments, the additional target analyte comprises one, two, three, four, five, six, seven, eight, nine, ten or more target analytes.

In some embodiments, the additional target analyte is a protein, a nucleic acid, a polysaccharide, a lipid, a cell, a fatty acid, a therapeutic agent, an organism, a virus, a toxin, a peptide, an oligosaccharide, a lipoprotein, a glycoprotein, a glycan, or a hormone.

In some embodiments, the method further comprises contacting the sample with (i) a plurality of beads comprising an additional capture moiety that specifically binds to the additional target analyte; (ii) an additional detecting moiety that binds to the additional target analyte, and (iii) an additional signal amplification moiety that binds to the additional detecting moiety to generate an additional detectable signal.

In another aspect, the invention provides a method of detecting a first target analyte and a second target analyte in a sample, the method comprising: (a) contacting a sample containing or suspected of containing the first target analyte and/or the second target analyte with (i) a plurality of first beads comprising a first capture moiety that specifically binds to the first target analyte, and (ii) a plurality of second beads comprising a second capture moiety that specifically binds to the second target analyte, under conditions and for a time sufficient for the first target analyte in the sample to bind to the first capture moiety and for the second target analyte in the sample to bind to the second capture moiety, wherein a plurality of the first beads are associated with zero first target analyte molecule; wherein a plurality of the first beads are associated with one first target analyte molecule; wherein at least about 20% of the first beads are associated with either zero or one first target analyte molecule; and wherein a plurality of the second beads are associated with zero second target analyte molecule; wherein a plurality of the second beads are associated with one second target analyte molecule; wherein at least about 20% of the second beads are associated with either zero or one second target analyte molecule; (b) contacting the product of step (a) with (i) a first detecting moiety that binds to the first target analyte, and (ii) a second detecting moiety that binds to the second target analyte; (c) contacting the product of step (b) with (i) a first signal amplification moiety that binds to the first detecting moiety to generate a first detectable signal for each bead carrying the first target analyte, and (ii) a second signal amplification moiety that binds to the second detecting moiety to generate a second detectable signal for each bead carrying the second target analyte; and (d) detecting the first detectable signal and the second detectable signal by flow cytometry, thereby detecting the first target analyte and the second target analyte in the sample.

In some embodiments, the first detectable signal and the second detectable signal are different signals, optionally, with different colors.

In some embodiments, the method further comprises detecting the first beads comprising the first capture moiety and the second beads comprising the second capture moiety by flow cytometry.

In some embodiments, the first beads comprising the first capture moiety and the second beads comprising the second capture moiety are different, optionally, with different colors, shapes, or sizes.

In some embodiments, the method reduces cross-reactivity or non-specific binding. In some embodiments, the cross-reactivity or non-specific binding is reduced by detecting the beads and the detectable signal by flow cytometry.

In some embodiments, the method has a limit of detection of about 0.1 aM to about 1 mM.

In some embodiments, the limit of detection is about 0.1 aM to about 1 mM, about 0.1 aM to about 1 μM, about 0.1 aM to about 1 nM, about 0.1 aM to about 1 μM, about 0.1 aM to about 1 fM, about 0.1 aM to about 900 aM, about 0.1 aM to about 800 aM, about 0.1 aM to about 700 aM, about 0.1 aM to about 600 aM, about 0.1 aM to about 500 aM, about 0.1 aM to about 400 aM, about 0.1 aM to about 300 aM, about 0.1 aM to about 200 aM, or about 0.1 aM to about 100 aM.

In some embodiments, the limit of detection is about 1 fM, about 900 aM, about 800 aM, about 700 aM, about 600 aM, about 500 aM, about 400 aM, about 300 aM, about 200 aM, about 100 aM, about 90 aM, about 80 aM, about 70 aM, about 60 aM, about 50 aM, about 40 aM, about 30 aM, about 20 aM, about 10 aM, or about 1 aM, or about 0.1 aM.

In some embodiments, the signal detection takes less than about one minute per sample, less than about 45 seconds per sample, or less than about 30 seconds per sample.

In some embodiments, the sample is contacted with about 2,000 to about 100,000 beads. In some embodiments, the sample is contacted with about 2,000 beads, about 5,000 beads, about 10,000 beads, about 20,000 beads, about 50,000 beads, or about 100,000 beads.

In some embodiments, the beads and the sample are incubated for about 1 min to about 48 h, about 1 min to about 10 h, or about 1 h to about 4 h. In some embodiments, the beads and the sample are incubated for about 10 minutes, about 20 minutes, about 30 minutes, about 40 minutes, about 50 minutes, about 1 h, about 2 h, about 3 h, about 4 h, or about 5 h.

Other features and advantages of the invention will be apparent from the following Detailed Description, the drawings, and the claims.

### BRIEF DESCRIPTION OF THE DRAWINGS

FIG. 1 depicts a schematic of an exemplary single molecule assay of the invention (molecular on-bead signal amplification for individual counting (MOSAIC)). Single target molecules are first captured with an excess number of antibody-coated paramagnetic beads, such that each bead carries either zero or one target molecule according to a Poisson distribution. Upon formation of single immunocomplex sandwiches with a biotinylated detector antibody and labeling with a streptavidin-DNA conjugate, rolling circle amplification is carried out to generate a long DNA concatemer attached to each immunocomplex. Fluorescently labeled DNA probes are hybridized to the RCA product, allowing “on” and “off” beads to be counted via flow cytometry.

FIGS. 2A-2D depict the analytical sensitivities of MOSAIC assays across different analytes and assay bead numbers. FIG. 2A provides the calibration curves for IL-10 MOSAIC assays using different assay bead numbers. The signal readout is denoted by average molecules per bead (AMB). FIG. 2B depicts the effect of MOSAIC assay bead number on the limit of detection (LOD) and signal-to-background for IL-10 detection. LOD values were calculated as three standard deviations above the background AMB. Relative signal-to-background for each assay bead number was determined as the signal-to-background of a specific calibrator normalized to the signal-to-background of the same calibrator using 100 000 assay beads. FIG. 2C provides the calibration curve for the corresponding IL-10 Simoa assay, using 100 000 assay beads and 400 000 helper beads. FIG. 2D depicts the effects of MOSAIC assay bead number on LOD and relative signal-to-background across additional analytes. All error bars represent the standard deviations of three replicates, with six replicates performed for the blank.

FIGS. 3A-3D depict that increasing sampling efficiency improves analytical sensitivity and precision. FIGS. 3A-3B set forth the limits of detection for calibration curves generated from subsets of varying bead numbers randomly sampled from MOSAIC calibration curves for IL-10 (FIG. 3A) and IFN-γ (FIG. 3B). Each color denotes the starting total assay bead number. Each point represents the median of 100 randomly sampled subsets, with error bars representing the interquartile range. Open circles denote values for which the upper quartile had a positive infinity value due to very high measurement coefficients of variation (CVs) at very low bead subset sizes. FIGS. 3C-3D provide the measurement CVs of the background signal for randomly sampled bead subsets of varying bead numbers for IL-10 (FIG. 3C) and IFN-γ (FIG. 3D).

FIGS. 4A-4D depict measurements of IFN-γ concentrations in human plasma and saliva using MOSAIC and Simoa. FIGS. 4A-4D provide the measured IFN-γ concentrations in 17 human plasma (FIG. 4A, FIG. 4B) and 26 saliva (FIG. 4C, FIG. 4D) samples between MOSAIC and Simoa assays. Concentrations shown represent the endogenous IFN-γ concentrations accounting for the four-fold dilution factor, and the limits of detection (LODs), and lower limits of quantification (LLOQs) also reflect the four-fold dilution factor. The Pearson correlation coefficients were 0.80 and 0.31 for the plasma and saliva (among detectable values) samples, respectively. The low correlation coefficient in saliva may be attributed to the small fraction of detectable samples using Simoa as well as multiple samples with IFN-γ levels near the LLOQ of Simoa or MOSAIC. Red dashed lines denote assay LODs, which were calculated as three standard deviations above the AMB of the background (buffer only). Samples with measurements below the assay LOD were assigned a value equal to the LOD. Error bars represent the standard deviation of two replicates.

FIGS. 5A-5D depict multiplexing with MOSAIC technology. FIG. 5A depicts the correlation of cytokine measurements in human plasma between a four-plex MOSAIC assay and the corresponding four-plex Simoa assay. Concentration, LOD, and LLOQ values reflect the four-fold dilution factor used in both assays. Samples with measurements below the assay LOD were assigned a value equal to the LOD. FIGS. 5B-5C provide the measured concentrations of eight protein analytes in human plasma (top) and saliva (bottom) using an eight-plex MOSAIC assay (FIG. 5B) and two four-plex Simoa assays (FIG. 5C). Concentrations shown are the measured concentration values in the 16-fold and 8-fold diluted plasma and saliva samples, respectively. Measurement below the assay LOD are assigned a value equal to the LOD and denoted by open symbols. FIG. 5D provides a schematic of multiplexing with MOSAIC. Beads coated with antibodies to different target analytes are encoded by using different fluorescent dyes with different wavelengths, intensities, and/or using multiple bead sizes. Upon capture of the single analyte molecules on each bead type, formation of single immunocomplex sandwiches, and labeling with streptavidin-DNA, rolling circle amplification is carried out and the mixture of beads is analyzed by flow cytometry. Beads are differentiated by a series of gates in different fluorescence channels, and the average molecules per bead for each bead type is then determined from the intensities in the fluorescence channel corresponding to the probe color.

FIGS. 6A-6E depict calibration curves for MOSAIC assays performed for individual cytokines across different assay bead numbers. Error bars represent the standard deviation of three to six replicates.

FIGS. 7A-7E depict calibration curves for Simoa assays performed for individual cytokines. Assay limits of detection are denoted by the red dashed lines. Error bars represent the standard deviation of three replicates, and six replicates of the blank.

FIGS. 8A-8C depict MOSAIC assays performed with four-hour target capture times. FIGS. 8A-8B provide the calibration curves for IL-10 (FIG. 8A) and IFN-γ (FIG. 8B) MOSAIC assays across different assay bead numbers with four-hour target capture times. Beads were incubated with sample for four hours, with detector antibody added during the last hour of target capture. Error bars represent the standard deviation of three replicates for each calibrator and six replicates for the blank. FIG. 8C sets forth the limits of detection and lower limits of quantification of IL-10 and IFN-γ MOSAIC assays with four-hour target capture times.

FIGS. 9A-9C depict MOSAIC and Simoa assays performed in human saliva. FIGS. 9A-9B provide the calibration curves for IFN-γ (FIG. 9A) and IL-12p70 (FIG. 9B) MOSAIC and Simoa assays performed in saliva, using StartingBlock™ Blocking Buffer (Thermo Fisher Scientific) as the diluent buffer. Assay limits of detection (LODs) for IFN-γ were 29.6 and 86.9 aM for MOSAIC and Simoa, respectively. Assay LODs for IL-12p70 were 44.2 and 304.3 aM for MOSAIC and Simoa, respectively. 10,000 assay beads were used for both MOSAIC assays, and 100,000 assay beads with 400,000 helper beads were used for the corresponding Simoa assays. FIG. 9C depicts the measured IL-12p70 concentrations in 26 saliva samples, using MOSAIC and Simoa. Concentrations and assay LODs, denoted by the red dashed lines, represent the assay measurements multiplied by the dilution factor.

FIGS. 10A-10D depict four-plex MOSAIC assay. FIGS. 10A-10B provide the calibration curves for four-plex MOSAIC (FIG. 10A) and Simoa (FIG. 10B) assays for IL-6, IL-1β, IL-10, and IFN-γ. FIG. 10C sets forth the limits of detection and lower limits of quantification of the four-plex MOSAIC and Simoa assays. FIG. 10D provides graphs of dropout curves for each analyte in the four-plex MOSAIC assay. Each graph shows the signal response of each bead with increasing amounts of the individual analytes in the multiplex assay, with error bars representing the standard deviation of duplicate measurements.

FIGS. 11A-11D depict representative calibration curves for eight-plex MOSAIC assay and two four-plex Simoa assays for IFN-γ, IL-10, IL-5, IL-6, IL-10, IL-12p70, IL-18, and VEGF. FIGS. 11A-11B provide representative eight-plex MOSAIC assay calibration curves obtained in the sample diluents used for plasma (FIG. 11A) and saliva (FIG. 11B) measurements. FIGS. 11C-11D provide the corresponding four-plex Simoa assay calibration curves obtained in the sample diluents used for plasma (FIG. 11C) and saliva (FIG. 11D) measurements. Error bars represent the standard deviation of three replicates, with six replicates performed for the blank.

FIG. 12 depicts dilution linearity of the eight-plex MOSAIC assay in three individual human plasma samples. The range of r² values from linear regression is shown for each analyte. For plasma #1, the 4× dilution factor measurement was excluded from linear regression, as there was poor dilution linearity between 4×and 8×dilutions. A final dilution factor of 16×was therefore selected for all reported plasma measurements to ensure consistent measurement accuracies. Error bars represent the standard deviation of duplicate measurements.

FIG. 13 depicts dropout curves for each analyte in the eight-plex MOSAIC assay. Each graph shows the signal response of each bead with increasing amounts of the individual analytes, with error bars representing the standard deviation of duplicate measurements.

FIGS. 14A-14B depict correlations of measurements by MOSAIC and Simoa in plasma (FIG. 14A) and saliva (FIG. 14B) for each of the analytes in the eight-plex MOSAIC assay. Two separate four-plex Simoa assays were performed as comparison. The limit of detection (LOD) and lower limit of quantification (LLOQ) values correspond to the effective LOD and LLOQ values accounting for the 16-fold dilution factor in the plasma samples. The Pearson correlation coefficient is shown for each analyte, determined from the measured concentrations of all samples. The weaker correlation between MOSAIC and Simoa for analytes whose concentrations were near or below the LLOQ of either method can be attributed to several factors, including lower measurement precision near the LLOQ or LOD. While we have validated the eight-plex MOSAIC assay via spike and recovery and dilution linearity experiments, differences in matrix effects on the MOSAIC and Simoa assays may have also contributed to weaker correlation at lower concentration ranges. Error bars represent the standard deviation of duplicate measurements.

FIGS. 15A-15D depict representative gating strategy used for identifying beads by flow cytometry for MOSAIC assays. Gating was performed using FlowJo™ software. Representative gates are shown for 488 multiplex beads (Quanterix Corp.). FIG. 15A depicts the gating strategy when an initial forward versus side scatter gate was applied to remove the majority of debris. FIG. 15B depicts the gating strategy when beads were further identified by fluorescence channels. FIG. 15C depicts the gating strategy when a final gate was applied to identify single beads. FIG. 15D provides representative scatterplots and histograms of fluorescence intensities for different IL-10 concentrations at 20,000 assay beads, using ATTO 647N-labeled DNA probes. Average molecule per bead (AMB) values were subsequently determined from the fluorescence intensity values using Python as described in the Examples.

FIG. 16 depicts dilution linearity of the eight-plex MOSAIC assay in three individual human saliva samples. The range of r² values from linear regression is shown for each analyte. For saliva #1, the 4× dilution factor measurement was excluded from linear regression, as there was poor dilution linearity between 4× and 8× dilutions. A final dilution factor of 8× was selected for all reported saliva measurements to ensure consistent measurement accuracies. Error bars represent the standard deviation of duplicate measurements.

FIG. 17 depicts the bead gating strategy used for eight-plex MOSAIC assay. A series of gates in different fluorescence channels were used to distinguish bead types and remove any aggregates consisting of different bead types. The gated populations highlighted in pink represent each bead type before further gating of single beads and subsequent analysis of fluorescence intensities in the probe channel.

FIG. 18 depicts the schematics of both non-barcoded multiplex MOSAIC and barcoded multiplex MOSAIC formats. For barcoded multiplex MOSAIC format, a unique DNA template is conjugated to each detector antibody in a multiplex MOSAIC assay and paired with a corresponding unique fluorescent dye-conjugated probe in the RCA reaction. In contrast, in the non-barcoded multiplex MOSAIC format, all detector antibodies are biotinylated and labeled with the same streptavidin-DNA conjugate and fluorescent probe, thus masking cross-reactive binding events.

FIG. 19A and FIG. 19B depict the dropout curves with increasing concentrations of each individual target protein for non-barcoded multiplex MOSAIC (FIG. 19A) and barcoded multiplex MOSAIC (FIG. 19B). The inclusion of only correct capture bead-probe color pairs in barcoded multiplex MOSAIC enables elimination of cross-reactive signals and, thus, providing more accurate multiplex measurements.

### DETAILED DESCRIPTION OF THE INVENTION

The invention provides methods and compositions for detection or measuring the concentration of a target analyte.

Quantitative and ultra-sensitive detection of protein biomarkers in minimally invasive biofluids such as blood or saliva has the potential to revolutionize medical diagnostics with earlier disease diagnoses, treatment monitoring, and disease reoccurrence monitoring. Techniques such as digital enzyme-linked immunosorbent assays (ELISA) and single molecule arrays (Simoa) allow for ultra-sensitive detection of low-abundance analytes, including proteins, nucleic acids, and other biologically relevant small molecules. However, these techniques still face limitations such as large and complex instrumentation, high cost, and complex workflows, making point-of-care applications impractical. Furthermore, there are still many potential disease biomarkers that exist below the detection limits of current digital detection methods.

The inventors of the present invention have developed an innovative, high-throughput, and highly accessible single molecule measurement platform that can detect low- to mid-attomolar protein concentrations. By addressing the challenge of low efficiencies in sampling rare target molecules in digital ELISA approaches, the sensitivity was enhanced by over tenfold over the current Simoa technology, which is presently the gold standard for ultrasensitive protein detection. The attomolar limits of detection (LODs) achieved by the present method correspond to at least 3-4 orders of magnitude improvement over conventional immunoassays. By localizing a non-diffusible fluorescent signal to each bead carrying a target molecule, this platform not only eliminates the need for bead loading into microwells or droplets for signal compartmentalization, but also enables significantly more beads to be analyzed for improved sampling efficiency and thereby enhances sensitivity.

This simple approach allows about 50-60% on average of the total beads to be analyzed—an about tenfold increase over the ˜5% sampling efficiency of the current Simoa technology. At low sample concentrations, particularly with capture efficiencies well below 100% (˜1-3% across all capture and labeling steps in the present assays developed in this work), improved sampling is critical for minimizing Poisson noise-associated measurement CVs. The significantly improved sampling efficiency of the present methods also allows the use of fewer assay beads compared to conventional Simoa, increasing the fraction of “on” beads” and thereby the signal to background. Further improvements in sensitivity can be attained by using affinity reagents with lower dissociation constants and decreasing nonspecific binding of the affinity reagents and streptavidin-DNA label. With the development of better affinity reagents and methods to reduce nonspecific binding, the present methods can potentially detect down to zeptomolar protein concentrations. With attomolar sensitivity, the present methods can pave the way towards discovery of new biomarkers and biological mechanisms underlying various diseases, such as tuberculosis.

Achieving an order of magnitude or more improvement in sensitivity with the present methods also holds important implications for the discovery of new blood-based biomarkers for many other cancer types and neurological disorders. A diagnostic blood test for neurodegenerative diseases such as Alzheimer's and Parkinson's diseases would prove especially critical for widespread screening and early diagnosis, which are currently very difficult due to the need for highly invasive lumbar punctures. In many cases where biomarker levels become detectable only after significant disease progression, the enhanced sensitivity of the present methods can accelerate disease diagnosis in early stages for improved health outcomes.

Importantly, the present methods also increase the simplicity of digital bioassay signal readout, which upon further development can potentially be integrated into a POC platform and thus address challenges of low sensitivity in current POC diagnostics. While increasing sampling efficiency for enhanced sensitivity in digital immunoassays has also been demonstrated in bead droplet arrays and droplet digital ELISA methods, these methods introduce additional complexity in fabrication and processing steps. In contrast, the readout process for the present methods requires only a flow cytometer, which is readily accessible by many research labs and can also be adapted to microfluidic systems at low costs, making the invention more amenable to rapid incorporation into existing laboratory infrastructures and point-of-care formats.

Furthermore, another key advantage of the present invention over previous digital ELISA methods, such as the dropcast single molecule assays, is the much faster, automated signal readout. Although the dropcast single molecule assays (dSimoa) enabled a bead dropcasting method for counting single molecules captured on the beads, these methods require long imaging times per sample to capture all the beads across multiple fields of view, thus limiting throughput. In contrast, the present invention allows for a much faster sample reading speed. The signal readout only takes about less than one minute per sample. Automation via the 96-well plate sampling modes that are already built into many benchtop flow cytometers further provides a streamlined workflow.

### Definitions

As used herein, the term “about” refers to a value that is within 10% above or below the value being described.

By “target analyte” is meant any atom, molecule, ion, molecular ion, compound, particle, cell, virus, complex, or fragment thereof to be either detected, measured, quantified, or evaluated. A target analyte may be contained in a sample (e.g., a liquid sample (e.g., a biological sample or an environmental sample)). Exemplary target analytes include, without limitation, a small molecule (e.g., an organic compound, a steroid, a hormone, a hapten, a biogenic amine, an antibiotic, a mycotoxin, an organic pollutant, a nucleotide, an amino acid, a monosaccharide, or a secondary metabolite), a protein (including a glycoprotein or a prion), a nucleic acid, a polysaccharide, a lipid, a fatty acid, a cell, a gas, a therapeutic agent, an organism (e.g., a pathogen), a virus, a toxin, a peptide, an oligosaccharide, a lipoprotein, a glycoprotein, a glycan, or a hormone. The target analyte may be naturally occurring or synthetic.

The term “small molecule,” as used herein, means any molecule having a molecular weight of less than 5000 Da. For example, in some embodiments, a small molecule is an organic compound, a steroid, a hormone, a hapten, a biogenic amine, an antibiotic, a mycotoxin, a cyanotoxin, a nitro compound, a drug residue, a pesticide residue, an organic pollutant, a nucleotide, an amino acid, a monosaccharide, or a secondary metabolite.

The terms “nucleic acid” and “polynucleotide,” as used interchangeably herein, refer to at least two covalently linked nucleotide monomers. The term encompasses, e.g., deoxyribonucleic acid (DNA), ribonucleic acid (RNA), hybrids thereof, and mixtures thereof. Nucleotides are typically linked in a nucleic acid by phosphodiester bonds, although the term “nucleic acid” also encompasses nucleic acid analogs having other types of linkages or backbones (e.g., phosphorothioate, phosphoramide, phosphorodithioate, O-methylphosphoroamidate, morpholino, locked nucleic acid (LNA), glycerol nucleic acid (GNA), threose nucleic acid (TNA), and peptide nucleic acid (PNA) linkages or backbones, and the like). The nucleic acids may be single-stranded, double-stranded, or contain portions of both single-stranded and double-stranded sequence. A nucleic acid can contain any combination of deoxyribonucleotides and ribonucleotides, as well as any combination of bases, including, for example, adenine, thymine, cytosine, guanine, uracil, and modified or non-canonical bases.

By “protein” herein is meant at least two covalently linked amino acids, which includes proteins, polypeptides, oligopeptides and peptides. The protein may be made up of naturally occurring amino acids and peptide bonds, or synthetic peptidomimetic structures. Thus “amino acid,” or “peptide residue,” as used herein, means both naturally occurring and synthetic amino acids. For example, homo-phenylalanine, citrulline and norleucine are considered amino acids for the purposes of the invention. The side chains may be in either the (R) or the (S) configuration. In some embodiments, the amino acids are in the (S) or L-configuration. If non-naturally occurring side chains are used, non-amino acid substituents may be used, for example to prevent or retard in vivo degradation. The term “portion” includes any region of a protein, such as a fragment (e.g., a cleavage product or a recombinantly-produced fragment) or an element or domain (e.g., a region of a polypeptide having an activity) that contains fewer amino acids than the full-length or reference polypeptide (e.g., about 5%, 10%, 15%, 20%, 30%, 40%, 50%, 60%, 70%, 80%, 90%, 95%, or 99% fewer amino acids).

The terms “bead,” “particle,” and “microsphere,” as used interchangeably herein, mean a small discrete particle. Suitable beads include, but are not limited to, paramagnetic beads, magnetic beads, non-magnetic beads, porous beads, plastic beads, ceramic beads, glass beads, polystyrene beads, methylstyrene beads, acrylic polymer beads, carbon graphited beads, titanium dioxide beads, latex or cross-linked dextrans such as SEPHAROSE beads, cellulose beads, nylon beads, cross-linked micelles, and TEFLON® beads. In some embodiments, spherical beads are used, but it is to be understood that non-spherical or irregularly-shaped beads may be used.

The term “capture moiety,” as used herein, means any molecule, particle, or the like that is capable of specifically binding a target analyte. A capture moiety may be conjugated, captured, attached, bound, or affixed to the bead. For example, in some embodiments, a capture moiety is an antibody (e.g., a full-length antibody (e.g., an IgG, IgA, IgD, IgE, or IgM antibody) or an antigen-binding antibody fragment (e.g., an scFv, an Fv, a dAb, a Fab, an Fab′, an Fab′₂, an F(ab′)2, an Fd, an Fv, or an Feb)), an aptamer, an antibody mimetic (e.g., an affibody, an affilin, an affimer, an affitin, an alphabody, an anticalin, an avimer, a DARPin, a fynomer, a Kunitz domain peptide, a monobody, or a nanoCLAMP), an antibody IgG binding protein (e.g., protein A, protein G, protein L, or recombinant protein A/G), a polypeptide, a nucleic acid, or a small molecule.

The term “detecting moiety” or “detection moiety” as used herein, means any molecule, particle, or the like that is capable of specifically binding to or otherwise specifically associating with a target analyte or another molecule that binds to or otherwise associates with the target analyte (e.g., a capture moiety). For example, in some embodiments, a detecting moiety is an antibody (e.g., a full-length antibody (e.g., an IgG, IgA, IgD, IgE, or IgM antibody) or an antigen-binding antibody fragment (e.g., an scFv, an Fv, a dAb, a Fab, an Fab′, an Fab′₂, an F(ab′)₂, an Fd, an Fv, or an Feb)), an aptamer, an antibody mimetic (e.g., an affibody, an affilin, an affimer, an affitin, an alphabody, an anticalin, an avimer, a DARPin, a fynomer, a Kunitz domain peptide, a monobody, or a nanoCLAMP), a molecularly-imprinted polymer, a receptor, a polypeptide, a nucleic acid, or a small molecule.

The term “signal amplification moiety” as used herein, means any molecule, particle, or the like that is capable of specifically binding to or otherwise specifically associating with a detecting moiety, and is capable of generating a detectable, e.g., amplified, signal that allows for detection. The signal can be any detectable signal, e.g., optically detectable labels such as fluorescent or chemiluminescent, or colorimetric labels, detectable by flow cytometry or optical assays, or can be any other label, e.g., gold beads or other label detectable by non-optical assays (e.g., using surface plasmon resonance or other methods). In some embodiments, the signal amplification moiety comprises an enzyme and/or a DNA molecule, e.g., a DNA primer and template, e.g., a concatemer or a long continuous DNA molecule that contains multiple copies of the same DNA sequence linked in series. The signal amplification moiety further comprises a probe with a detectable label, e.g., a fluorescently labeled DNA probe, which is capable of hybridizing to the concatemer generated.

A first moiety “specifically binds” (or grammatical variants thereof) a second moiety if the first moiety (e.g., a capture moiety) binds to the second moiety with specificity sufficient to differentiate between the second moiety (e.g., a target analyte) and other components or contaminants of the test sample. The binding is generally sufficient to remain bound under the conditions of the assay, including wash steps to remove non-specific binding, although in some embodiments, wash steps are not desired; i.e., for detecting low affinity binding partners. In some embodiments, a first moiety specifically binds to a second moiety with an equilibrium dissociation constant (K_{D}) of about 10⁻⁵ M, 10⁻⁶ M, 10⁻⁷M, 10⁻⁸⁷ M, 10⁻⁹ M, 10⁻¹⁰M, 10⁻¹¹ M, 10⁻²M, 10⁻¹³M, 10⁻¹⁴M, 10⁻¹⁵ M, or lower.

The term “cross reactivity” refers to the non-specific binding of the beads comprising the capture moiety for the target analyte with a non-target analyte, e.g., a different analyte, or a contaminant, or other components of the test sample.

The term “non-covalent affinity binding pair” refers to a pair of moieties, e.g., a first member and a second member, that bind and form a non-covalent complex. Exemplary non-covalent affinity binding pairs include, without limitation, biotin-biotin binding protein (e.g., biotin-streptavidin and biotin-avidin), ligand-receptor, antigen-antibody or antigen binding fragment, hapten-anti-hapten, immunoglobulin (Ig) binding protein-Ig, dioxigenin, SNAP-tag, CLIP-tag, or other complementary binding partners. The members of a non-covalent affinity binding pair may have any suitable binding affinity. For example, the members of an affinity binding pair may bind with an equilibrium dissociation constant (K_{D} or Kd) of about 10⁻⁵ M, 10⁻⁶ M, 10⁻⁷ M, 10⁻⁸ M, 10⁻⁹ M, 10⁻¹⁰ M, 10⁻¹¹ M, 10⁻¹²M, 10⁻¹³ M, 10⁻¹⁴M, 10⁻¹⁵ M, or lower.

A “pathogen” is an agent that can cause a disease or illness to its host, including, without limitation, a virus (e.g., a parvovirus (e.g., an adeno-associated virus (AAV)), a retrovirus (e.g., a lentivirus (e.g., human immunodeficiency virus (HIV))), a herpesvirus, an adenovirus, and the like), a bacterium (e.g., Mycobacterium tuberculosis, or E. coli), a protozoon, a fungus, or a prion.

As used herein, “subject” means any animal. In one embodiment, the subject is a human. Other animals that can be subjects include but are not limited to non-human primates (e.g., monkeys, gorillas, and chimpanzees), domesticated animals (e.g., horses, pigs, donkeys, goats, rabbits, sheep, cattle, yaks, alpacas, and llamas), and companion animals (e.g., cats, lizards, snakes, dogs, fish, hamsters, guinea pigs, rats, mice, and birds).

As used herein, “biomarker” and “marker” interchangeably refer to an analyte (e.g., a small molecule, DNA, RNA, protein, carbohydrate, or glycolipid-based molecular marker), the expression or presence of which in a subject's sample can be detected by methods described herein and is useful, for example, for determining a prognosis, or for monitoring the responsiveness or sensitivity of a mammalian subject to a therapeutic agent.

As used herein, “biological sample” refers to any biological sample obtained from or derived from a subject, including body fluids, body tissue (e.g., tumor tissue), cells, or other sources. Body fluids are, e.g., lymph, whole blood (including fresh, frozen, or dried and rehydrated), plasma (including fresh, frozen, or dried and rehydrated), serum (including fresh, frozen, or dried and rehydrated), a blood fraction containing peripheral blood mononuclear cells, urine, saliva, semen, sweat, lacrimal fluid, synovial fluid, cerebrospinal fluid, feces, mucous, vaginal fluid, and spinal fluid. Samples also include breast tissue, liver tissue, pancreatic tissue, cervix tissue, renal tissue, colonic tissue, brain tissue, muscle tissue, synovial tissue, skin, hair follicle, bone marrow, tumor tissue, a tissue lysate or homogenate, or an organ lysate or homogenate. Methods for obtaining tissue biopsies and body fluids from mammals are well known in the art.

By “environmental sample” is meant any sample that is obtained from an environment, e.g., a water sample, soil sample, air sample, extraterrestrial materials, and the like. An environmental sample may contain biological molecules or organisms.

### Methods of the Invention

The invention provides methods of detecting a target analyte in a sample. The methods can also involve measuring a concentration of a target analyte in a sample.

In one aspect, the invention provides a method of detecting a target analyte in a sample. The method comprises (a) contacting a sample containing or suspected of containing the target analyte with a plurality of beads comprising a capture moiety that specifically binds to the target analyte, under conditions and for a time sufficient for the target analyte in the sample to bind to the capture moiety, wherein a plurality of the beads are associated with zero target analyte molecule; wherein a plurality of the beads are associated with one target analyte molecule; and wherein at least about 20% of the beads are associated with either zero or one target analyte molecule; (b) contacting the product of step (a) with a detecting moiety that binds to the target analyte, (c) contacting the product of step (b) with a signal amplification moiety that binds to the detecting moiety to generate a detectable signal for each bead carrying the target analyte; and (d) detecting the detectable signal by flow cytometry, thereby detecting the target analyte in the sample.

The sample containing or suspected of containing the target analyte is first contacted with a plurality of beads conjugated to capture moieties that are capable of specifically binding to an analyte of interest, under conditions that allow binding of the analyte to the beads to form a bead-analyte complex. Once the bead-analyte complex is formed, the methods include contacting the analyte with a detecting moiety and a signal amplification moiety, and generating an on-bead, non-diffusible detectable signal, e.g., a fluorescent signal, which allows for detection of each bead carrying a single target analyte by flow cytometry. By localizing the signal on the beads, the present methods eliminate the need for signal compartmentalization into microwells or droplets.

In some embodiments, steps (a), (b), (c) of the methods of the present invention, or any combination thereof, are performed sequentially or simultaneously.

In some embodiments, at least about 20%, 30%, 40%, 50%, 60%, 70%, 80%, 90%, 91%, 92%, 93%, 94%, 95%, 96%, 97%, 98% or 99% of the beads, are associated with either zero or one target analyte molecule.

The method of the present invention also includes measuring the concentration of a target analyte in a sample. In some embodiments, the concentration is proportional, e.g., directly proportional or inversely proportional, to an intensity level of at least one signal indicative of the presence of a target analyte.

In some embodiments, the method of the invention has a limit of detection of about 0 mM to about 5 mM, e.g., about 0.1 aM to about 5 mM, about 0.1 aM to about 4 mM, about 0.1 aM to about 3 mM, about 0.1 aM to about 2 mM, about 0.1 aM to about 1 mM, about 0.1 aM to about 1 μM, about 0.1 aM to about 1 nM, about 0.1 aM to about 1 μM, about 0.1 aM to about 1 fM, about 0.1 aM to about 900 aM, about 0.1 aM to about 800 aM, about 0.1 aM to about 700 aM, about 0.1 aM to about 600 aM, about 0.1 aM to about 500 aM, about 0.1 aM to about 400 aM, about 0.1 aM to about 300 aM, about 0.1 aM to about 200 aM, or about 0.1 aM to about 100 aM. In some embodiments, the limit of detection of the method of the present invention is about 1 fM, about 900 aM, about 800 aM, about 700 aM, about 600 aM, about 500 aM, about 400 aM, about 300 aM, about 200 aM, about 100 aM, about 90 aM, about 80 aM, about 70 aM, about 60 aM, about 50 aM, about 40 aM, about 30 aM, about 20 aM, about 10 aM, about 1 aM, or about 0.1 aM.

The methods of the present invention may further include detecting or measuring a concentration of one or more additional target analyte(s) in the sample. In some embodiments, the methods may include detecting or measuring a concentration of about 2, about 3, about 4, about 5, about 6, about 7, about 8, about 9, about 10, about 11, about 12, about 14, about 16, about 18, about 20, or more different target analytes.

In some embodiments, the additional target analyte is a small molecule, a protein, a nucleic acid, a polysaccharide, a lipid, a cell, a fatty acid, a therapeutic agent, an organism, a virus, a toxin, a peptide, an oligosaccharide, a lipoprotein, a glycoprotein, a glycan, or a hormone. The additional target analyte may be any suitable target analyte as described herein or known in the art. In some embodiments, the methods further include contacting the sample with (i) a plurality of beads with an additional capture moiety that specifically bind to the additional target analyte; (ii) an additional detecting moiety that binds to the additional target analyte, and (iii) an additional signal amplification moiety that binds to the additional detecting moiety to generate an additional detectable signal.

In one aspect, the present invention provides a method of detecting a first target analyte and a second target analyte in a sample, the method comprising: (a) contacting a sample containing or suspected of containing the first target analyte and/or the second target analyte with (i) a plurality of first beads comprising a first capture moiety that specifically binds to the first target analyte, and (ii) a plurality of second beads comprising a second capture moiety that specifically binds to the second target analyte, under conditions and for a time sufficient for the first target analyte in the sample to bind to the first capture moiety and for the second target analyte in the sample to bind to the second capture moiety, wherein a plurality of the first beads are associated with zero first target analyte molecule; wherein a plurality of the first beads are associated with one first target analyte molecule; wherein at least about 20% of the first beads are associated with either zero or one first target analyte molecule; and wherein a plurality of the second beads are associated with zero second target analyte molecule; wherein a plurality of the second beads are associated with one second target analyte molecule; wherein at least about 20% of the second beads are associated with either zero or one second target analyte molecule; (b) contacting the product of step (a) with (i) a first detecting moiety that binds to the first target analyte, and (ii) a second detecting moiety that binds to the second target analyte; (c) contacting the product of step (b) with (i) a first signal amplification moiety that binds to the first detecting moiety to generate a first detectable signal for each bead carrying the first target analyte, and (ii) a second signal amplification moiety that binds to the second detecting moiety to generate a second detectable signal for each bead carrying the second target analyte; and (d) detecting the first detectable signal and the second detectable signal by flow cytometry, thereby detecting the first target analyte and the second target analyte in the sample.

In another aspect, the present invention provides a method of detecting a first target analyte, a second target analyte and a third or more target analyte in a sample, the method comprising: (a) contacting a sample containing or suspected of containing the first target analyte, the second target analyte and/or the third or more target analyte with (i) a plurality of first beads comprising a first capture moiety that specifically binds to the first target analyte, (ii) a plurality of second beads comprising a second capture moiety that specifically binds to the second target analyte, and (iii) a plurality of third or more beads comprising a third or more capture moiety that specifically binds to the third or more target analyte, under conditions and for a time sufficient for the first target analyte in the sample to bind to the first capture moiety, for the second target analyte in the sample to bind to the second capture moiety, and for the third or more target analyte in the sample to bind to the third or more capture moiety, wherein a plurality of the first beads are associated with zero first target analyte molecule; wherein a plurality of the first beads are associated with one first target analyte molecule; wherein at least about 20% of the first beads are associated with either zero or one second target analyte molecule; wherein a plurality of the second beads are associated with zero second target analyte molecule; wherein a plurality of the second beads are associated with one second target analyte molecule; wherein at least about 20% of the second beads are associated with either zero or one second target analyte molecule; and wherein a plurality of the third beads are associated with zero third target analyte molecule; wherein a plurality of the third beads are associated with one third target analyte molecule; wherein at least about 20% of the third beads are associated with either zero or one third target analyte molecule; (b) contacting the product of step (a) with (i) a first detecting moiety that binds to the first target analyte, (ii) a second detecting moiety that binds to the second target analyte, and (iii) a third or more detecting moiety that binds to the third or more target analyte; (c) contacting the product of step (b) with (i) a first signal amplification moiety that binds to the first detecting moiety to generate a first detectable signal for each bead carrying the first target analyte, (ii) a second signal amplification moiety that binds to the second detecting moiety to generate a second detectable signal for each bead carrying the second target analyte, and (iii) a third or more signal amplification moiety that binds to the third or more detecting moiety to generate a third or more detectable signal for each bead carrying the third or more target analyte; and (d) detecting the first detectable signal, the second detectable signal, and the third or more detectable signal by flow cytometry, thereby detecting the first target analyte, the second target analyte, and the third or more target analyte in the sample.

Any of the preceding methods may involve providing a prognosis or a diagnosis of a disease, e.g., tuberculosis, for a subject based on the concentration of the one or more target analyte(s) in the sample. Any of the preceding methods may involve selecting a therapy for a patient based on the concentration of the one or more target analyte(s) in the sample. Any of the preceding methods may involve treating a subject with a therapy based on the concentration of the one or more target analyte(s) in the sample. For example, in a particular embodiment, the methods may be used to prognose or diagnose tuberculosis, a disease for which the ability to detect and diagnose at an early stage is critical to prevent development of active tuberculosis and the spread of the disease.

In some embodiments, the methods of the invention include detecting and/or quantifying one or more target analytes, e.g., one or more molecules expressed by Mycobacterium tuberculosis in a sample. In other embodiments, the methods of the present invention include a prognosis or a diagnosis of tuberculosis for a subject based on the presence and/or concentration of one or more target analytes in a sample. The methods may also involve selecting a therapy or treating a subject with a therapy based on the presence and/or concentration of one or more target analytes in a sample.

### Beads

The present methods include the use of micro or nanoparticle beads conjugated to capture moieties that bind to a desired analyte. The beads can be made of various materials. In general, any polymeric or plastic materials can be used to create the microparticles, microbeads, or nanoparticles, including materials such as polystyrene and polyethylene. In some embodiments, microparticles can be formed of biologically-compatible polymer materials such as polyacrylates, polymethacrylates, and/or polyamides. In certain embodiments, metallic, metal-oxide, semiconductor, and/or semiconductor oxide micro- and/or nanoparticles formed from one or more of Au, Ag, Pt, Al, Cu, Ni, Fe, Cd, Se, Ge, Pd, Sn, iron oxide, TiO₂, Al₂O₃, and SiO₂ can be made in many sizes and used. For example, monocrystalline iron oxide nanoparticles (MIONs) and crosslinked iron oxide (CLIO) particles can be used. Any suitable beads can be used in the context of the invention, including, without limitation, magnetic beads, paramagnetic beads, non-magnetic beads, porous beads, plastic beads, ceramic beads, glass beads, polystyrene beads, methylstyrene beads, acrylic polymer beads, carbon graphited beads, titanium dioxide beads, latex or cross-linked dextrans such as SEPHAROSE beads, cellulose beads, nylon beads, cross-linked micelles, and TEFLON® beads. In certain embodiments, the bead is a paramagnetic bead. In some embodiments, the beads can have different shapes, sizes and/or colors. In some embodiments, the beads comprising capture moiety for one analyte have a different shape, size and/or color than beads for a different analyte, such that the beads can be detected or distinguished, e.g., by flow cytometry, to eliminate non-specific binding or cross reactivity. In some embodiments, spherical beads are used, but non-spherical or irregularly-shaped beads may be used. In some embodiments, the beads comprise or are conjugated to a detectable signal or a label, e.g., a fluorescent label, a chemiluminescent label, or a colorimetric label. In some embodiments, the detectable signal or label may be detected by flow cytometry or optical assays, or any other detection methods described herein or known in the art. The color of the labels, e.g., fluorescent labels, on the beads may be different for each different analyte to be detected.

In any of the preceding methods, the bead is a magnetic bead (e.g., a paramagnetic bead). In some embodiments, the beads have a size (e.g., a diameter) of about 0.01 μm to about 10 μm, e.g., about 0.01 μm, about 0.1 μm, about 0.2 μm, about 0.3 μm, about 0.4 μm, about 0.5 μm, about 0.6 μm, about 0.7 μm, about 0.8 μm, about 0.9 μm, about 1 μm, about 1.5 μm, about 2 μm, about 2.5 μm, about 3 μm, about 3.5 μm, about 4 μm, about 4.5 μm, about 6 μm, about 6.5 μm, about 7 μm, about 7.5 μm, about 8 μm, about 8.5 μm, about 9 μm, about 9.5 μm, or about 10 μm. In some embodiments, the beads have a size of about 1 μm to about 5 μm, about 1 μm to about 4 μm, about 1 μm to about 3 μm, or about 1 μm to about 2 μm.

Any of the preceding methods may involve contacting the sample with about 1,000 to about 5,000,000 beads, e.g., about 1000, about 2000, about 5000, about 10,000, about 20,000, about 30,000, about 40,000, about 50,000, about 60,000, about 70,000, about 80,000, about 90,000, about 100,000, about 200,000, about 300,000, about 400,000, about 500,000, about 600,000, about 700,000, about 800,000, about 900,000, about 1,000,000, about 2,000,000, about 3,000,000, about 4,000,000, or about 5,000,000 beads. In some embodiments, the method may involve contacting the sample with about 2,000 to about 100,000 beads, about 10,000 to about 5,000,000 beads, about 10,000 to about 4,000,000 capture probes, about 10,000 to about 3,000,000 beads, about 10,000 to about 2,000,000 capture probes, about 10,000 to about 1,000,000 beads, about 10,000 to about 500,000 capture probes, about 10,000 to about 400,000 beads, about 10,000 to about 300,000 beads, about 10,000 to about 200,000 beads, or about 10,000 to about 100,000 beads. In some embodiments, the method may involve contacting the sample with about 2,000 beads, about 5,000 beads, about 10,000 beads, about 20,000 beads, about 50,000 beads, or about 100,000 beads. In some embodiments, the method may involve contacting the sample with about 20,000 beads.

The sensitivity and/or the sampling efficiency of the methods of the present invention can be adjusted based on the number of beads. In some embodiments, reducing the bead number can improve the sensitivity of the methods. In other embodiments, increasing the bead number improves the sensitivity of the methods. The number of beads used can also be optimized for each analyte being detected.

Capture Moiety, Detecting Moiety and Signal Amplification Moiety The beads for use in the methods of the present invention are coated with e.g., conjugated to, capture moieties that can specifically bind to an analyte of interest. Once the capture moiety binds to the target analyte, the bead-analyte complex is formed. A detecting moiety and a signal amplification moiety are used to generate an on-bead, non-diffusible detectable signal for each bead carrying a single target analyte.

The capture and/or detecting moieties are capable of specifically binding to or otherwise specifically associating with a target analyte. Any suitable capture and/or detecting moiety may be used in the context of the present invention. For example, in some embodiments, the capture moiety and/or the detecting moiety can be an antibody, an aptamer, an antibody mimetic, a polypeptide, a nucleic acid, a molecularly-imprinted polymer, a receptor, a binding protein, or a small molecule. The antibody may be a full-length antibody (e.g., an IgG, IgA, IgD, IgE, or IgM antibody) or an antigen-binding antibody fragment (e.g., an scFv, an Fv, a dAb, a Fab, an Fab′, an Fab′₂, an F(ab′)₂, an Fd, an Fv, or an Feb). The antibody mimetic may be wherein the antibody mimetic is an affibody, an affilin, an affimer, an affitin, an alphabody, an anticalin, an avimer, a DARPin, a fynomer, a Kunitz domain peptide, a monobody, or a nanoCLAMP.

In some embodiments, the capture and/or detecting moiety is an antibody or antigen-binding portion thereof or an aptamer that binds to the analyte, e.g., wherein the analyte is a protein or peptide.

In some embodiments, the capture and/or detecting moiety is an oligonucleotide that is complementary to a portion of a nucleic acid of interest. In some embodiments, the capture and/or detecting moiety is a ligand-binding portion of a protein, e.g., of a receptor, wherein the analyte is a molecule such as a hormone.

The method further comprises an on-bead signal amplification step. Specifically, the method comprises contacting the sample with a signal amplification moiety that binds to the detecting moiety in order to generate a detectable signal which allows for detection of each bead carrying a single target analyte. The signal can be any detectable signal. In some embodiments, the signal comprises optically detectable labels such as fluorescent or chemiluminescent, or colorimetric labels detectable by flow cytometry or optical assays.

In some embodiments, the signal amplification moiety comprise an enzyme, e.g., a phi29 DNA polymerase, and/or a nucleic acid molecule, e.g., a DNA primer and template, e.g., a concatemer or a long continuous DNA molecule that contains multiple copies of the same DNA sequence linked in series. The signal amplification moiety further comprises a probe with a detectable label, e.g., a fluorescently labeled DNA probe or a ratiometric combination of fluorescently labeled DNA probes, which is capable of hybridizing to the concatemer generated. In some embodiments, the detectable labels comprise different colors for different target analytes. In some embodiments, the different target analytes can be detected by using combinations of different beads, e.g., bead with different colors, fluorescent intensity, shapes or sizes, and different detectable labels, e.g., labels with different colors or different ratios of colors, which can be distinguished by flow cytometry.

In some embodiments, the method reduces cross-reactivity or non-specific binding, e.g., by detecting the beads and the detectable signal by flow cytometry. In some embodiments, a unique pair of fluorescent label-encoded capture bead and DNA template-conjugated detector antibody is used for each analyte. Each unique DNA template-conjugated antibody, when amplified during the RCA reaction, is labeled with a specific fluorescent dye-conjugated DNA probe or ratiometric combination of dye-conjugated DNA probes. Each probe color or ratiometric combination of colors can be distinguished using multiple detection channels in flow cytometry. Upon capture of single analyte molecules on beads, a mixture of detector antibodies conjugated with unique primer-template sequences is added, followed by washing and resuspension in an RCA reaction that contains a mixture of fluorescent dye-conjugated DNA probes. Each analyte will correspond to a unique pair of (1) capture bead color, fluorescence intensity, or size; and (2) fluorescent probe color or ratio of colors. As a result, only the “correct” matched pairs of capture bead and probe signal will be classified as “on” beads for each analyte, whereas the “wrong” pairs of capture bead and probe colors, i.e., cross-reactive binding events, would be eliminated from further analysis.

In some embodiments, the detectable signal is generated by rolling circle amplification (RCA). For RCA, a streptavidin label or a detector antibody is conjugated with a DNA primer and template. The generated DNA concatemer attached to each bead-analyte complex can be hybridized with a large number of complementary fluorescently labeled DNA probes for detection. In these methods, the sensitivity can be tuned by increasing or decreasing RCA time.

Other nucleic acid amplification methods can also be used to generate the on-bead signal, e.g., hybridization chain reaction, loop-mediated isothermal amplification, radical polymerization, Enzyme-catalyzed proximity labeling (PL) polymerization (see, e.g., Branon et al., Nat Biotechnol. 2018, 36(9):880-887); polymerization-based signal amplification (e.g., visible-light-induced polymerization, e.g., as described in Badu-Tawiah et al., Lab Chip, 2015, 15, 655); magnetic bead-quantum dot immunoassays (Kim et al., ACS Sens. 2017, 2, 6, 766-772); or immunosignal hybridization chain reaction (isHCR) (Lin et al., Nat Methods. 2018 April; 15(4):275-278). Branched DNA assays can also be used, as described in Dunbar and Das, J Clin Virol. 2019; 115: 18-31.

Alternatively, the amplified on-bead signal can be generated using a proximal labeling methods, such as tyramide signal amplification (TSA), biotin ligase, and engineered ascorbate peroxidase. TSA, also referred to as Catalyzed Reporter Deposition (CARD), is a highly sensitive method enabling the detection of analytes present in low abundance. TSA is used in immunohistochemistry and in situ hybridization experiments, and has been used for digital ELISA (Akama et al., Anal. Chem. 2016, 88 (14), 7123-7129). In TSA, HRP (e.g., bound to a second binding moiety) catalyzes the conversion of labeled tyramide into a reactive radical which then covalently binds to nearby tyrosine residues, generating a high-density detectable signal.

In some embodiments, the methods include contacting the detecting moiety with a pre-amplified signal such as a labeled polymer or nanoparticle or nucleic acid concatemers; see, e.g. Tang et al., Analyst, 2013, 138, 981-990; Hansen et al., Anal Bioanal Chem. 2008; 392(1-2): 167-175; Wu et al., Chem 2017, 2, 760-790; Gormley et al., Nano Lett. 2014, 14, 11, 6368-6373 (radicals generated by either enzymes or metal ions are polymerized to form polymers that entangle multiple gold nanoparticles (AuNPs)); Melnychuk and Klymchenko, J. Am. Chem. Soc. 2018, 140, 34, 10856-10865 (dye-loaded polymeric nanoparticles.)

In some embodiments, the detecting moiety is linked directly to the signal amplification moiety. In some embodiments, the detecting moiety and the signal amplification moiety are linked indirectly, e.g., by a non-covalent affinity binding pair, wherein the detecting moiety is linked to a first member of the non-covalent affinity binding pair, and the signal amplification moiety is linked to a second member of the non-covalent affinity binding pair. In some embodiments, the non-covalent affinity binding pair is biotin-streptavidin, biotin-avidin, dioxigenin, SNAP-tag, CLIP-tag, ligand-receptor, antigen-antibody, or antibody binding protein-antibody, or any other complementary binding partners known in the art. In some embodiments, the detecting moiety is attached a biotin molecule, and the signal amplification moiety, e.g., a DNA concatemer, is conjugated to a streptavidin molecule.

### Detection

The present invention utilizes on-bead signal generation from single captured target molecules to enable signal molecule counting by flow cytometry. By localizing a non-diffusible fluorescent signal to each bead carrying a target molecule, this method allows “on” and “off” beads to be counted by flow cytometry in a rapid manner.

The present invention differs from past digital detection methods in several ways: (1) it does not require bead isolation into individual containers such as microwells or droplets for signal compartmentalization, as the signal is localized to each bead, thus eliminating the requirement for complex microfabrication or droplet generation; (2) it increases sampling efficiency of rare target molecules as it is able to count a higher percentage of beads than the current Simoa technology (at least 30% compared to ˜5%), thus improving the limit of detection by about an order of magnitude; (3) flow cytometry is readily accessible by many research labs and can also be adapted to microfluidic systems at low costs, making the invention more amenable to rapid incorporation into existing laboratory infrastructures and point-of-care formats.

In addition, the invention differs from other flow cytometry-based immunoassays such as Luminex assays: (1) it achieves digital detection by capturing single target molecules on beads instead of multiple target molecules per bead as in existing flow cytometry-based immunoassays, thus enabling much lower detection limits by several orders of magnitude; (2) it uses signal amplification to yield a detectable signal for each individual target molecule, rather than relying on fluorescently-labeled secondary antibodies which require multiple target molecules per bead to generate a detectable signal by flow cytometry.

Another key advantage of the present invention over previous digital ELISA methods is the much faster, automated signal readout. Automation via the 96-well plate sampling modes that are already built into many benchtop flow cytometers further provides a streamlined workflow.

In some embodiments, the signal detection takes less than about five minute per sample. In some embodiments, the signal detection takes less than about four minute per sample. In some embodiments, the signal detection takes less than about three minute per sample. In some embodiments, the signal detection takes less than about two minute per sample. In some embodiments, the signal detection takes less than about one minute per sample. In some embodiments, the signal detection takes less than about 30 seconds per sample.

The on-bead signal generated by the methods of the present invention can be detected and/or quantified, and the detection and/or quantification can be related to the presence, the quantity, and/or the concentration of target analytes in the sample being tested.

In some embodiments, the on-bead signal is detected by a flow cytometer. A flow cytometer is an apparatus of supplying a particle suspension into a narrow tube, separating the suspended particles one by one, irradiating laser on each particle and detecting and measuring the fluorescence emission from the particle excited by the laser and the scattering of the laser by the particle.

The flow cytometer for use in the present invention is not particularly limited, and any conventional flow cytometer may be used as it is. It has, for example, a laser source (e.g., He/Ne or argon) emitting a single-wavelength light in one or more detection regions. Laser sources emitting lights at different wavelengths may be installed in the different detection regions. The laser may be converged by using a beam-shaping lens.

Before irradiation with the laser, beads are focused into a single-file line. In a traditional flow cytometer, a sheath fluid (e.g., IsoFlow sheath fluid (trade name, manufactured by Beckmann Coulter), Dako Sheath Fluid (trade name, manufactured by Dako Japan, etc.)), and a sample solution containing a target analyte are introduced into a flow cell, where the sheath fluids and the sample solution form laminar flow without mutual mixing. During flow in the flow cell, the sample solution flows, while forming a sample core as it is held between the sheath fluids, and is irradiated by laser when it passes through a laser irradiation zone. However, the beads may also be aligned using Dean focusing, viscoelastic focusing, physical confinement, or other methods.

In some embodiments, the flow cytometer allows for acquisition of the detection data for a single target molecule. In other embodiments, the flow cytometer allows for simultaneous measurement of multiple targets in a single measurement operation. In some embodiments, the one or more target analytes can be detected by using combinations of different beads, e.g., beads with different colors, shapes or sizes, and different detectable labels, e.g., labels with different colors, that can be distinguished by flow cytometry.

The flow cytometer for use in the present invention may be a commercially available flow cytometer such as FACS Calibur (manufactured by Becton, Dickinson and Company), PERFLOW Ana (manufactured by The Furukawa Electric Co., Ltd.), EPICS ALTRA (manufactured by Beckmann Coulter), CyAn (manufactured by Dako Japan), or JSAN manufactured by Bay bioscience Co., Ltd.). “Flow cytometer” is also understood to include other devices which reproduce some or all features of a flow cytometer sufficiently well to allow detection of beads and on-bead signal.

### Target Analytes

As would be appreciated by a person of ordinary skill in the art, a large number of target analytes can be detected and, optionally, quantified using the methods of the invention. Any suitable target analyte can be investigated using the methods of the invention. The target analytes listed below are provided as non-limiting examples. The target analyte may be naturally occurring or synthetic.

In some embodiments, the target analyte comprises, without limitation, a protein (e.g., an antibody, a cytokine (e.g., an interleukin (e.g., IL-1, IL-2, IL-3, IL-4, IL-5, IL-6, IL-7, IL-7, IL-9, IL-10, IL-11, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-35, or IL-36), a lymphokine, a monokine, an interferon (IFN, e.g., IFN-beta and IFN-gamma), a colony stimulating factor (e.g., CSF, G-CSF, GM-CSF, and the like), a chemokine, a tumor necrosis factor (TNF, including TNF-alpha and TNF-beta), a bone morphogenetic protein (BMP), and the like), a receptor (e.g., an interleukin receptor, a receptor tyrosine kinase, and the like), a ligand, an enzyme (e.g., a polymerase, a cathepsin, a calpain, an aminotransferase (e.g., aspartate aminotransferase (AST) or alanine aminotransferase (ALT)), a protease (e.g., a caspase), a lipase, an oxidoreductase, a kinase, nucleotide cyclases, a transferase, a hydrolase, a lyase, an isomerase, and the like), or a prion), a nucleic acid (e.g., DNA or RNA, e.g., microRNA), a polysaccharide, a lipid, a cell (e.g., a prokaryotic cell (e.g., a bacterium (e.g., Mycobacterium tuberculosis, or E. coli)) or a eukaryotic cell (e.g., a fungal cell or a human cell), including tumor cells), a fatty acid, a glycoprotein, a biomolecule, a therapeutic agent (e.g., an antibody, a fusion protein (e.g., an Fc fusion protein), a cytokine, a soluble receptor, and the like), an organism (e.g., a pathogen), a virus (e.g., a parvovirus (e.g., an adeno-associated virus (AAV)), a retrovirus, a herpesvirus, an adenovirus, a lentivirus, and the like), or a small molecule. In some embodiments, the target analyte may be a molecule expressed by a bacterium, e.g., Mycobacterium tuberculosis. In some embodiments, the target analyte may be post-translationally modified (e.g., phosphorylated, methylated, glycosylated, ubiquitinated, and the like).

In some embodiments, the target analyte has a molecular weight of at least about 1000 Da, e.g., at least about 1500 Da, at least about 2000 Da, at least about 2500 Da, at least about 3000 Da, at least about 3500 Da, at least about 4000 Da, at least about 4500 Da, or at least about 5000 Da.

In some embodiments, the target analyte may have one or more binding sites that can be recognized by the capture moiety and the detecting moiety. The target analyte may be detected in a sandwich assay format based on the methods of the present invention. In some embodiments, the beads may be associated with either zero or one target analyte molecule, or the beads may be associated with one or more, e.g., 1, 2, 3, 4, or 5, or more target analyte molecules.

Any of the methods described herein may further include detecting and, optionally, quantifying a target analyte, for example, in a multiplexed assay. For example, in some embodiments, the methods may include detecting and, optionally, quantifying, about 1, about 2, about 3, about 4, about 5, about 6, about 7, about 8, about 9, about 10, about 11, about 12, about 14, about 16, about 18, about 20, or more different target analytes. In some embodiments, the one or more target analytes can be detected by using combinations of different beads, e.g., beads with different colors, shapes or sizes, and different detectable labels, e.g., labels with different colors, that can be distinguished by flow cytometry.

In some embodiments, the target analyte may be post-translationally modified (e.g., phosphorylated, methylated, glycosylated, ubiquitinated, and the like).

In any of the preceding methods, the concentration of the target analyte in the sample can range from about 0 mM to about 5 mM, e.g., about 0.1 aM to about 5 mM, about 0.1 aM to about 4 mM, about 0.1 aM to about 3 mM, about 0.1 aM to about 2 mM, about 0.1 aM to about 1 mM, about 0.1 aM to about 1 μM, about 0.1 aM to about 1 nM, about 0.1 aM to about 1 μM, about 0.1 aM to about 1 fM, about 0.1 aM to about 900 aM, about 0.1 aM to about 800 aM, about 0.1 aM to about 700 aM, about 0.1 aM to about 600 aM, about 0.1 aM to about 500 aM, about 0.1 aM to about 400 aM, about 0.1 aM to about 300 aM, about 0.1 aM to about 200 aM, or about 0.1 aM to about 100 aM.

In any of the preceding methods, the incubating time for the beads and the sample can be adjusted to enhance the signal-to-background ratio and analytical sensitivity. In some embodiments, the incubating can be performed for about 1 min to about 48 h, about 1 min to about 10 h, or about 1 h to about 4 h. In some embodiments, the incubating time is about 1 min, about 5 min, about 10 min, about 20 min, about 30 min, about 40 min, about 50 min, about 60 min, about 2 h, about 3 h, about 4 h, about 5 h, about 6 h, about 7 h, about 8 h, about 9 h, about 10 h, about 11 h, about 12 h, about 13 h, about 14 h, about 15 h, about 16 h, about 17 h, about 18 h, about 19 h, about 20 h, about 21 h, about 22 h, about 23 h, about 24 h, about 25 h, about 26 h, about 27 h, about 28 h, about 29 h, about 30 h, about 40 h, or about 48 h.

In some embodiments, the beads and the sample are incubated for about 10 minutes, about 20 minutes, about 30 minutes, about 40 minutes, about 50 minutes, about 1 h, about 2 h, about 3 h, about 4 h, or about 5 h. In one embodiment, the incubating time is about 1 hour.

### Samples

Any suitable sample may be used in the context of the present invention. For example, in some embodiments, the sample is a liquid sample (e.g., a biological sample or an environmental sample). Exemplary biological samples include, without limitation, body fluids, body tissue (e.g., tumor tissue), cells, or other sources. Exemplary body fluids include, without limitation, e.g., lymph, whole blood (including fresh or frozen), plasma (including fresh or frozen), serum (including fresh or frozen), a blood fraction containing peripheral blood mononuclear cells, urine, saliva, semen, sweat, lacrimal fluid, synovial fluid, cerebrospinal fluid, feces, mucous, vaginal fluid, and spinal fluid. Samples also include breast tissue, liver tissue, pancreatic tissue, cervix tissue, lung tissue, renal tissue, colonic tissue, brain tissue, muscle tissue, synovial tissue, skin, hair follicle, bone marrow, tumor tissue, a tissue lysate or homogenate, and an organ lysate or homogenate. Methods for obtaining tissue biopsies and body fluids from mammals are well known in the art. In other embodiments, the sample may be an environmental sample, e.g., a water sample, soil sample, air sample, extraterrestrial materials, or the like.

The volume of the fluid sample analyzed may potentially be any amount within a wide range of volumes, depending on a number of factors such as, for example, the number of capture probes used/available, the number of detection probes, and the like. As non-limiting examples, the sample volume may be about 0.01 μl, about 0.1 μl, about 1 μl, about 5 μl, about 10 μl, about 100 μl, about 1 ml, about 5 ml, about 10 ml, or the like. In some cases, the volume of the fluid sample is between about 0.01 μl and about 10 ml, between about 0.01 μl and about 1 ml, between about 0.01 μl and about 100 μl, or between about 0.1 μl and about 10 μl.

In some embodiments, the fluid sample may be diluted prior to use in a method described herein. For example, in embodiments where the source of an analyte molecule is a body fluid (e.g., blood, plasma, or serum), the fluid may be diluted with an appropriate solvent (e.g., a buffer such as PBS buffer). A fluid sample may be diluted about 1-fold, about 2-fold, about 3-fold, about 4-fold, about 5-fold, about 6-fold, about 10-fold, about 50-fold, about 100-fold, or greater, prior to use. The sample may be added to a solution comprising the plurality of capture probes or detectable moieties, or the plurality of capture probes or detectable moieties may be added directly to or as a solution to the sample.

### Compositions

The invention provides compositions which can be used in the detection and, optionally, quantification of target analytes in a sample. For example, the invention provides a composition that includes: (a) a bead (e.g., a paramagnetic bead), the bead being linked to a plurality of capture moiety (e.g., an antibody), (b) a detecting moiety (e.g., an antibody), the detection moiety being linked to a first member of a non-covalent affinity binding pair (e.g., a biotin moiety); and (c) a signal amplification moiety (e.g., a phi29 DNA polymerase, a DNA primer, a DNA concatemer, and a fluorescently labeled probe), the signal amplification moiety (e.g., the DNA concatemer) being linked to a second member of the non-covalent affinity binding pair (e.g., a streptavidin moiety), wherein the detecting moiety is bound to one of the target analytes captured by the capture moiety on the bead, and the signal amplification moiety is bound to the detecting moiety by binding of the first member of the non-covalent affinity binding pair to the second member of the non-covalent affinity binding pair.

### Kits

The invention provides kits for detecting or measuring a concentration of a target analyte in a fluid sample. The kit may include, for example, a plurality of beads (e.g., paramagnetic beads). The plurality of beads provided may have a variety of properties and parameters, as described herein. The plurality of beads may be coated with capture moieties that specifically bind to a target analyte. The plurality of beads may also be conjugated to a detectable signal or label, e.g., a fluorescent label, for detection. The kit may also include a detecting moiety that can specifically bind to the target molecule once the target molecule is captured by the capture moiety on the bead. The kit may further include a signal amplification moiety that can generate an on-bead non-diffusible signal which is detectable by flow cytometry. The capture moiety, the detecting moiety and the signal amplification moiety provided may have a variety of properties and parameters, as described herein.

The kit may also include a reaction vessel for collecting the sample with the detectable signal. The reaction vessels may be configured to receive and contain the beads in the sample.

The kits and articles of manufacture described herein may be configured for carrying out any of the methods or assays as described herein, e.g., in the Examples.

In some embodiments, the kit may include instructions for use of components described herein. That is, the kit can include a description of use of the beads and reaction vessels, for example, for use with a system to determine a measure of the concentration of target analyte(s) in a fluid sample. As used herein, “instructions” can define a component of instruction and/or promotion, and typically involve written instructions on or associated with packaging of the invention. Instructions also can include any oral or electronic instructions provided in any manner such that a user of the kit will clearly recognize that the instructions are to be associated with the kit. Additionally, the kit may include other components depending on the specific application, as described herein.

### EXAMPLES

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### Example 1: MOSAIC: High-Throughput, High-Multiplex Digital Protein Detection with Attomolar Sensitivity

A major challenge in many clinical diagnostic applications is the measurement of low abundance proteins and other biomolecules in biological fluids such as plasma or saliva. The advent of digital technologies such as the digital enzyme-linked immunosorbent assay (ELISA) has enabled 1000-fold increases in sensitivity over conventional protein detection methods. However, current digital ELISA technologies still possess insufficient sensitivities for biomarkers that exist below their detection limits, and require specialized instrumentation or time-consuming workflows that have limited their widespread implementation. To address these challenges, the present inventors have developed a more sensitive, streamlined, and rapid digital ELISA platform, Molecular On-bead Signal Amplification for Individual Counting (MOSAIC), that attains low attomolar limits of detection, with an order of magnitude enhancement in sensitivity over these other methods. MOSAIC uses a rapid, automatable flow cytometric readout that vastly increases throughput and is easily integrated into existing laboratory infrastructure. As MOSAIC provides high sampling efficiencies for rare target molecules, assay bead number can readily be tuned to enhance signal to background with high measurement precision. Furthermore, the solution-based signal readout of MOSAIC expands the number of bead types that can simultaneously be analyzed for higher order multiplexing with femtomolar sensitivities or below, in contrast to microwell or droplet-based digital methods. As proof-of-principle demonstrations, MOSAIC was applied towards improving the detectability of low abundance cytokines in saliva and ultrasensitive multiplexed measurements of eight protein analytes in plasma. The attomolar sensitivity, rapid throughput, and broad multiplexing capabilities of MOSAIC provide a highly accessible and versatile ultrasensitive platform that can potentially accelerate protein biomarker discovery as well as diagnostic testing for diverse disease applications.

### Development of the MOSAIC Platform.

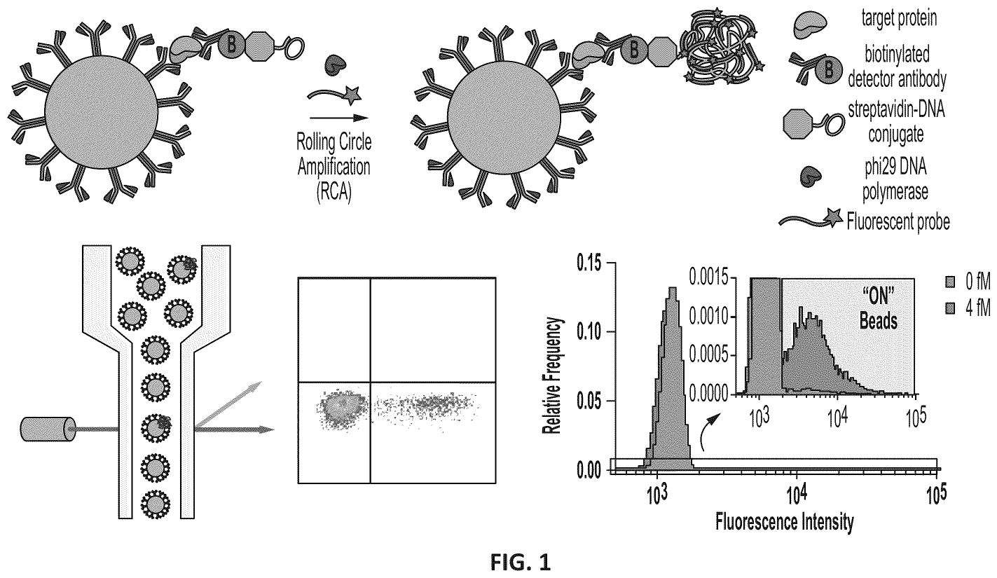

To establish an ultrasensitive digital ELISA platform with a rapid flow cytometric signal readout, a method for generating a localized fluorescent signal from single target molecules captured on an excess number of antibody-coated beads was utilized (FIG. 1).⁶ Upon formation of single immunocomplex sandwiches on the beads and labeling with streptavidin conjugated to a DNA primer-template pair, rolling circle amplification (RCA) is carried out to form a DNA concatemer attached to each immunocomplex sandwich. The incorporation of a fluorescently labeled DNA probe in the RCA reaction enables in situ hybridization to the concatemer, thus producing a strong fluorescent signal on each bead carrying a target molecule. As the amplified signal is attached to each immunocomplex sandwich, individual beads are subsequently analyzed by flow cytometry for counting of fluorescent “on” and “off” beads.

To evaluate the feasibility of single molecule counting using this readout method, MOSAIC was first applied towards the detection of interleukin-10 (IL-10), an anti-inflammatory cytokine with diverse roles across cancers, autoimmune diseases, and infectious diseases.^{12,13} The “on” beads spanned a wide range of fluorescence intensities due to the heterogeneous size distribution of RCA concatemers. The cutoff value for distinguishing “on” from “off” beads was determined by fitting a two-Gaussian mixture model to the fluorescence intensity values using expectation maximization.¹⁴ At lower concentrations, almost all beads fell into the lower (“off”) Gaussian, and beads were counted as “on” if they were more than five standard deviations above the mean of this Gaussian. At higher concentrations, labels were predicted for the beads based on the likelihood that they were drawn from the “off” or “on” Gaussian. Using this algorithm, the inventors were able to consistently quantify the average molecule per bead (AMB), a measure of the fraction of “on” beads.

### High Sampling Efficiencies in MOSAIC Enable Improved Sensitivities.

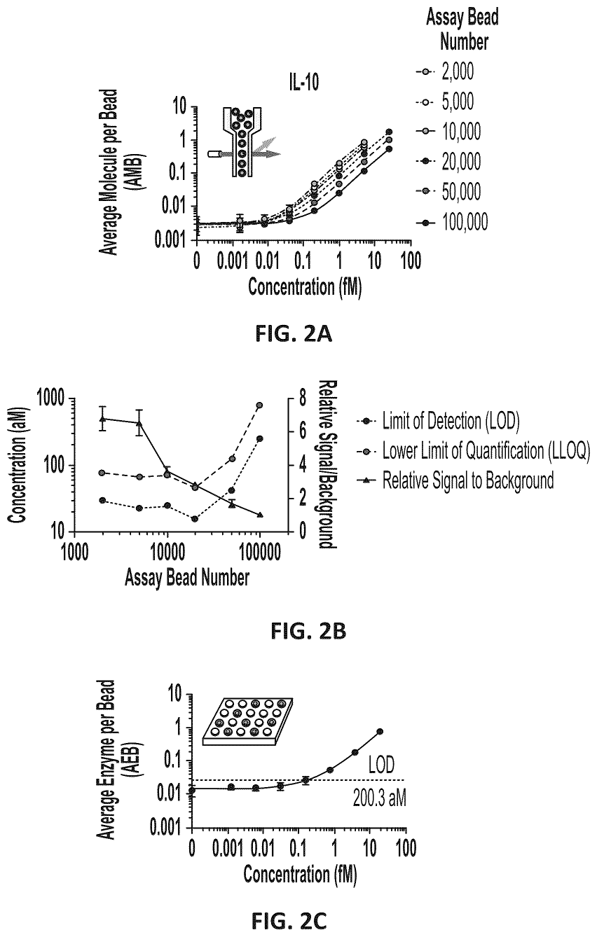

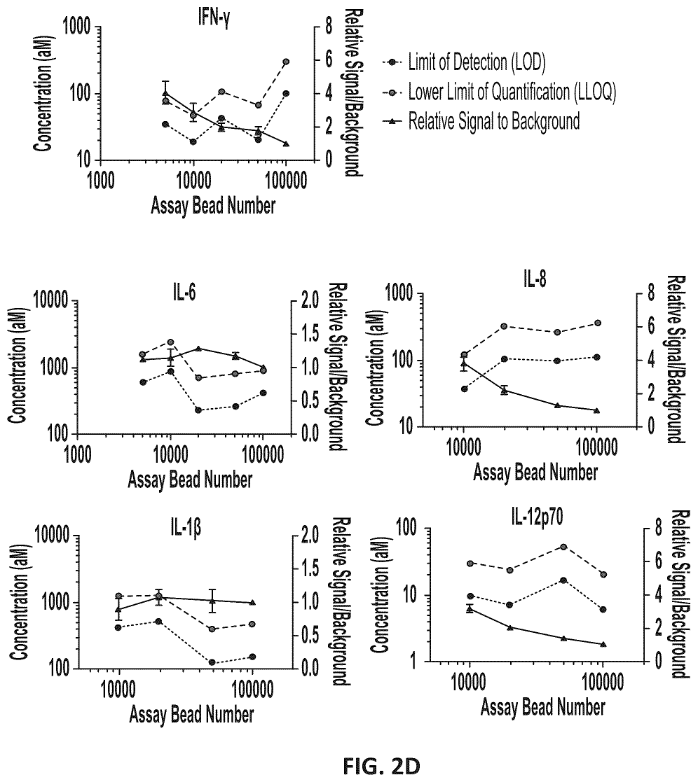

Importantly, because nearly the entire bead solution can be analyzed by flow cytometry, MOSAIC attains high sampling efficiencies. Approximately 50-60% of the total initial assay beads are analyzed per sample, representing an over tenfold increase in sampling efficiency over Simoa. The analytical sensitivity of MOSAIC may be maximized by reducing the number of total assay beads, increasing the ratio of target molecules to beads and thereby signal-to-background ratio, while maintaining sufficient measurement precision. To exploit the possibility of enhancing sensitivity using lower bead numbers in MOSAIC, assay bead number was systematically varied from 100,000 to 2,000 for IL-10. The signal-to-background ratio improved with decreasing bead number (FIG. 2A). To quantify the improvement in signal-to-background, the signal-to-background ratio of the 0.2 fM calibrator was normalized in each calibration curve to that of the 100,000-bead calibration curve. This relative signal-to-background increased as the number of beads was reduced, and a corresponding improvement in the limit of detection (LOD) was observed, with approximately an order of magnitude enhancement in sensitivity compared to 100,000 beads (FIG. 2B). As bead number was further reduced to 2,000 beads, however, the analytical sensitivity did not improve further, due to increasing effects of Poisson noise on measurement precision at very low bead numbers. Maximal sensitivity was attained at 20,000 beads, with an LOD of 15.9 aM, representing an over 12-fold enhancement in sensitivity over the corresponding Simoa assay (FIG. 2C and Table 1). While standard Simoa assays typically use 500,000 total assay beads, for a closer comparison to MOSAIC, 100,000 assay beads were used with 400,000 helper beads for improved signal to background, despite fewer total beads analyzed.

TABLE 1
Limit of detection (LOD) and lower limit of quantification
(LLOQ) values of optimized MOSAIC assays and the corresponding
Simoa assays for various analytes. LOD and LLOQ values
are calculated as three and ten standard deviations,
respectively, above the background.

| | MOSAIC | Limit of | | Lower Limit of | |
|---|---|---|---|---|---|
| | Assay | Detection (aM) | | Quantification (aM) | |
| | Bead | MOSAIC | Simoa | MOSAIC | Simoa |
| Analyte | Number | (3×) | (3×) | (10×) | (10×) |
| IL-10 | 20,000 | 15.9 | 200.3 | 45.9 | 753.3 |
| IFN-γ | 10,000 | 18.9 | 125.1 | 47.0 | 441.8 |
| IL-6 | 20,000 | 227.9 | 661.2 | 699.0 | 1965.5 |
| IL-1β | 50,000 | 124.0 | 690.2 | 401.5 | 2032.8 |
| IL-8 | 10,000 | 37.9 | 179.9 | 120.2 | 639.1 |
| IL-12p70 | 100,000 | 5.8 | 69.6 | 19.9 | 217.6 |

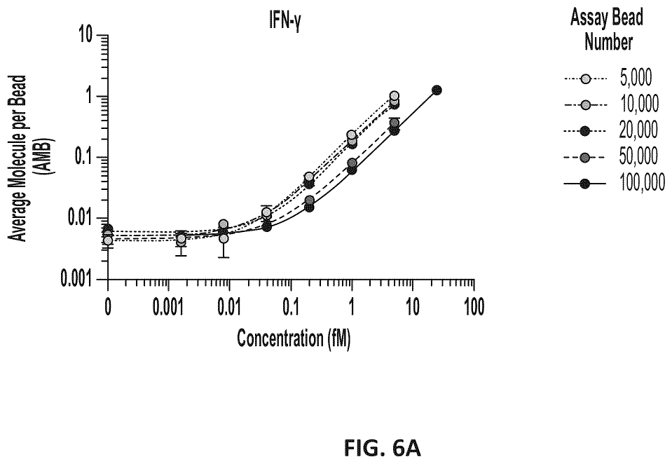

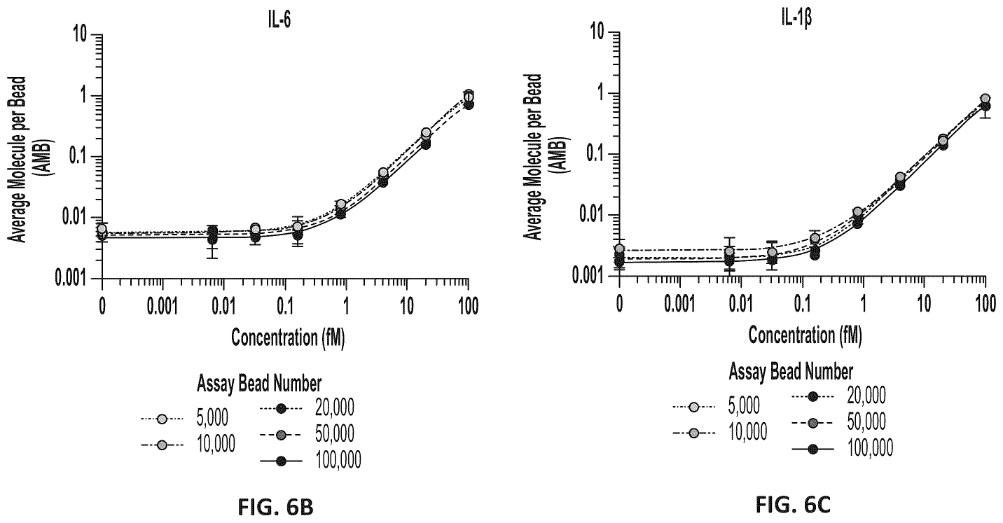

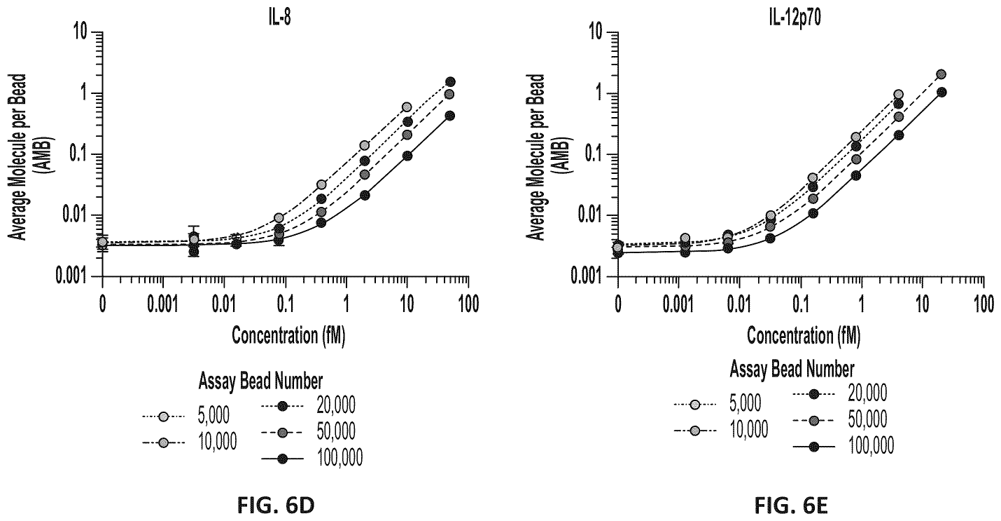

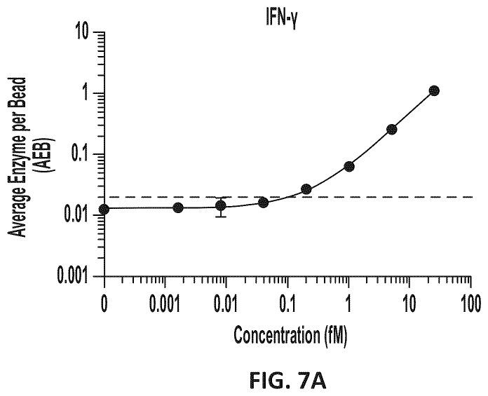

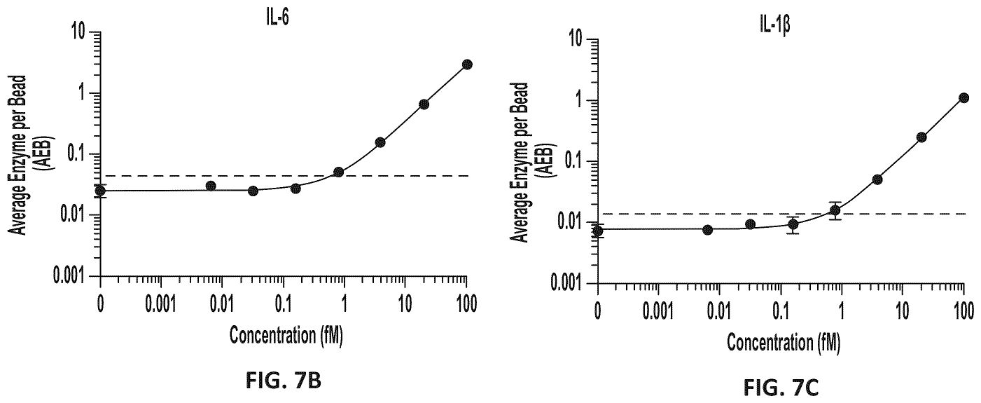

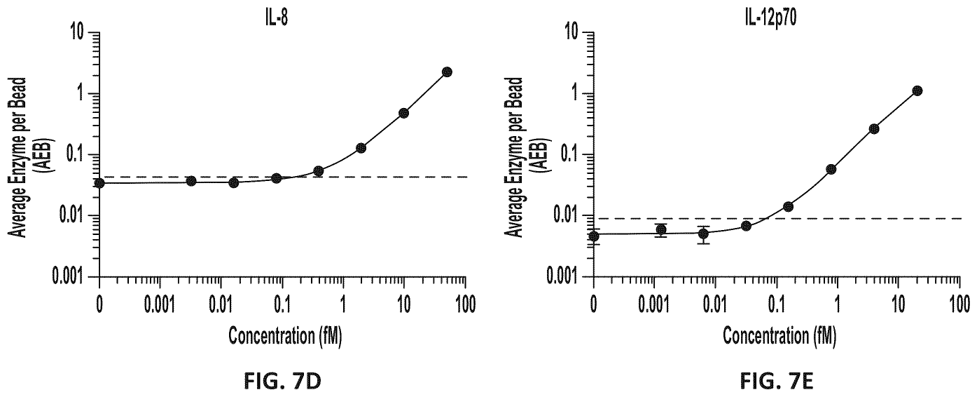

To assess whether reducing bead number can similarly enhance the sensitivity of MOSAIC for other analytes, the MOSAIC platform was expanded to additional cytokines and performed similar variations of bead number for each analyte (FIG. 2D, FIGS. 6A-6E, and Table 2). Improved signal-to-background upon decreasing bead number from 100,000 to 10,000 was also observed for interferon-gamma (IFN-γ), interleukin-8 (IL-8), and interleukin-12 p70 (IL-12p70), with optimal sensitivity achieved at 10,000 beads for IFN-γ and IL-8. For IL-12p70, despite the increased signal-to-background, sensitivity remained similar across all bead numbers, with the lowest LOD of 5.8 aM attained using 100,000 assay beads. For IL-6 and IL-10, there was little increase in the signal-to-background as bead number was reduced from 100,000. Consistent with this observation, the LOD did not improve much with decreasing assay bead number. It was observed that only three- to five-fold improvements in sensitivity over the corresponding Simoa assays for these analytes, as expected from the lack of signal-to-background enhancement; these improvements can be attributed to increased sampling efficiencies. Our results demonstrate that MOSAIC achieves 3-12 times higher sensitivity over Simoa, the current state-of-the-art for ultrasensitive protein detection (Table 1 and FIGS. 7A-7E). Due to the much higher sampling efficiencies of MOSAIC compared to standard digital ELISA, much lower bead numbers down to 10,000 beads can be used to enhance the signal-to-background for several analytes, while maintaining sufficient measurement precision. While the extent of signal-to-background improvement is dependent upon the particular antibody pair, assay bead number can be readily optimized for each analyte.

TABLE 2
Analytical sensitivities for all single-plex MOSAIC assays.
Limits of detection (LODs) and lower limits of quantification (LLOQs) of
MOSAIC assays performed for all cytokines across different assay bead numbers.

| Assay | | | | | | |
|---|---|---|---|---|---|---|
| Bead | IL-10 | | IFN-γ | | IL-6 | |
| Number | LOD (aM) | LLOQ (aM) | LOD (aM) | LLOQ (aM) | LOD (aM) | LLOQ (aM) |
| 100,000 | 255.5 | 809.1 | 99.9 | 304.5 | 421.3 | 900.1 |
| 50,000 | 43.3 | 124.7 | 20.7* | 67.4* | 263.7 | 809.9 |
| 20,000 | 15.9 | 45.9 | 43.1 | 107.5 | 227.9* | 699.0* |
| 10,000 | 24.8 | 72.1 | 18.9 | 47.0 | 877.1* | 2,447.0* |
| 5,000 | 22.7 | 67.6 | 34.8 | 78.0 | 607.6 | 1,578.2 |
| 2,000 | 30.1 | 77.3 | — | — | — | — |
| Assay | | | | | | |
| Bead | IL-1β | | IL-8 | | IL-12p70 | |
| Number | LOD (aM) | LLOQ (aM) | LOD (aM) | LLOQ (aM) | LOD (aM) | LLOQ (aM) |
| 100,000 | 152.0 | 467.9 | 109.9 | 359.8 | 5.8 | 19.9 |
| 50,000 | 124.0 | 401.5 | 98.8 | 262.2 | 16.7 | 52.9 |
| 20,000 | 517.1 | 1,280.4 | 105.2 | 322.4 | 6.9 | 23.5 |
| 10,000 | 425.5 | 1,236.4 | 37.9 | 120.2 | 9.6 | 30.5 |

To test the reproducibility of the MOSAIC assays, identical calibration curves were measured on different days for a few select curves, which resulted in similar AMB values and LODs. As a result, the average LODs of the two separate calibration curves are reported for these curves. As further confirmation of the reproducibility of these assays, the LODs obtained from combining all replicates were also assessed among separate curves into a single calibration curve, which resulted in similar values as the reported average LOD values.

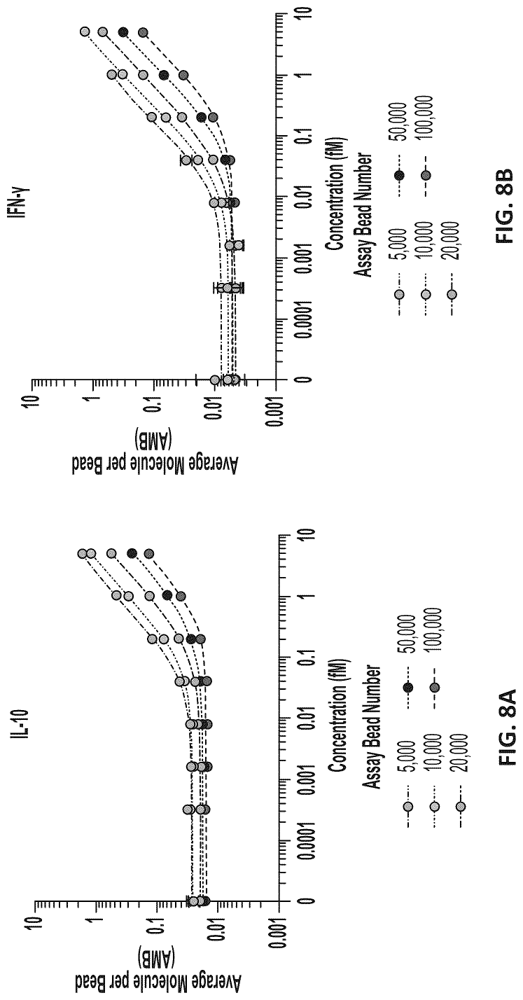

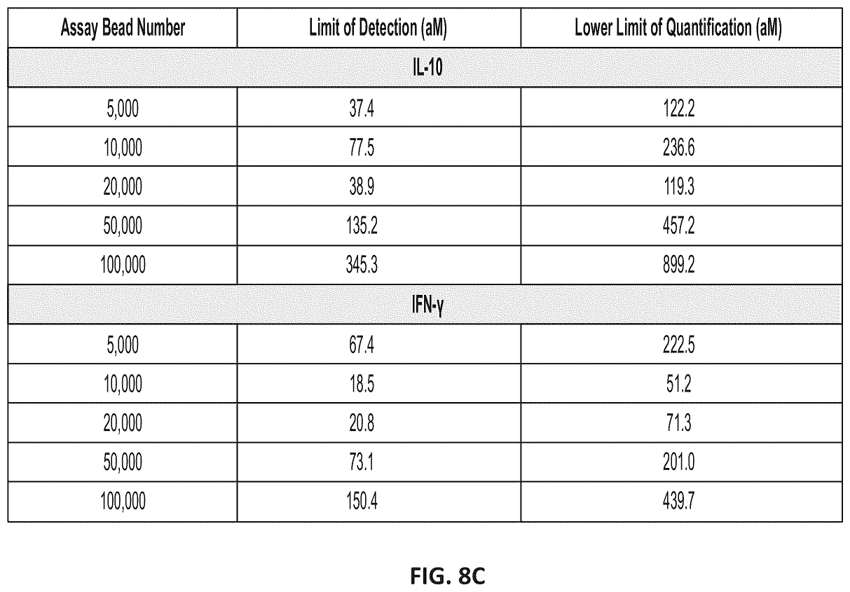

As decreasing the number of assay beads also reduces the number of capture antibody molecules present, capture kinetics may be slower. It was thus investigated whether longer target capture times could further enhance the signal-to-background ratio and analytical sensitivity. For both IL-10 and IFN-γ, however, increasing target capture times from one hour to four hours resulted in similar or slightly worse LODs (FIGS. 8A-8C). Therefore, one hour target capture times was used in subsequent experiments for a more efficient workflow.

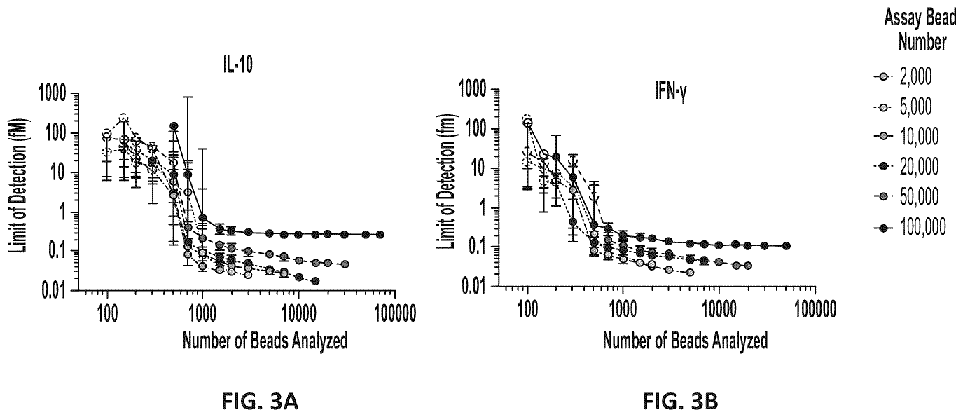

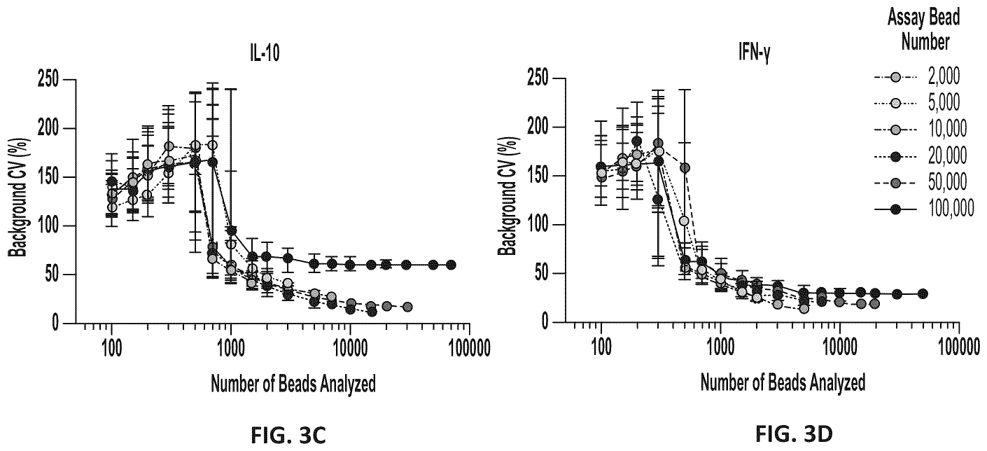

To determine the extent of sensitivity improvement arising from improved sampling efficiencies in MOSAIC, random sampling of subsets of beads was performed among the calibration curves obtained for IL-10 and IFN-γ and examined the effects of number of beads analyzed on sensitivity and precision. Consistent with the high Poisson noise that occurs at low event numbers, the LODs obtained when only a few hundred beads were analyzed were very high, across all starting assay bead numbers (FIGS. 3A-B). For each subset size, random sampling was repeated 100 times, and high variation in calculated LODs among replicate subsets was observed when 1,000 or fewer beads were analyzed, in line with the poor precision and reproducibility expected at these low sampling efficiencies. As an increasing percentage of assay beads are analyzed, analytical sensitivities improve considerably, with smaller gains in sensitivity as the number of analyzed beads increases past several thousand. Furthermore, the improvement in sensitivity with increasing sampling efficiency corresponded to a decrease in the measurement coefficients of variation of the background signal (FIGS. 3C-3D). The results thus empirically support the important role of improved sampling efficiencies in the enhanced sensitivity of MOSAIC.

### MOSAIC Improves Detectability of Low-Abundance Analytes in Biological Fluids.

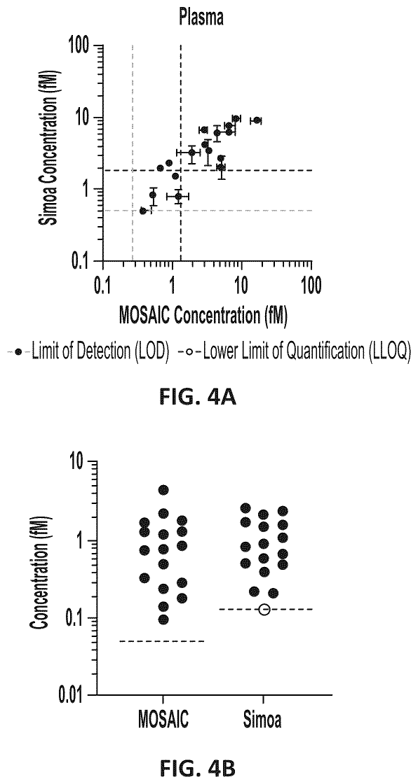

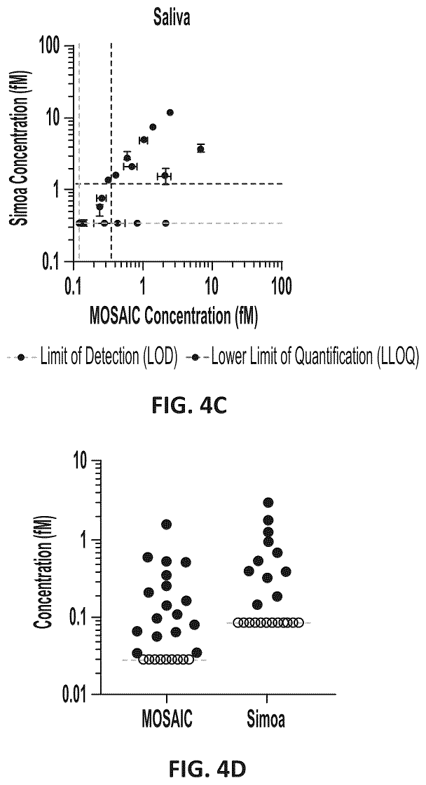

Next, the performance of MOSAIC was explored in biological fluids to see whether its enhanced sensitivity can improve the detectability of low abundance biomarkers. As a representative cytokine, IFN-γ was measured using both MOSAIC and the corresponding Simoa assay in a cohort of human plasma samples (FIGS. 4A-4B). The IFN-γ concentrations measured by MOSAIC, using 10,000 assay beads, showed good correlation with those measured by Simoa, supporting the accuracy of the MOSAIC assay. Furthermore, although IFN-γ was generally detectable in plasma using both methods, one out of the 17 plasma samples remained undetectable by Simoa, while the more sensitive MOSAIC assay achieved 100% detectability.

To further assess whether the superior sensitivity of MOSAIC can improve the detectability of low abundance analytes in biological fluids, MOSAIC was applied to saliva. As saliva contains a minimal serum component filtered from blood through the salivary glands, many potential biomarkers exist at much lower levels in saliva than in blood, necessitating ultrasensitive techniques. As a proof of concept, MOSAIC was used to measure the levels of IFN-γ in a cohort of saliva samples. While IFN-γ was detectable in only 42% (11/26) of the saliva samples using Simoa, the enhanced sensitivity of the low-bead MOSAIC assay improved the detectability of IFN-γ to over 65% (17/26) of the saliva samples (FIGS. 4C-4D). All saliva samples with detectable IFN-γ levels using Simoa were detectable by MOSAIC, with positively correlated measurements. The wider variability between MOSAIC and Simoa measurements in saliva compared to plasma may be attributed to differing matrix interference effects with the different assays. However, recoveries within an acceptable range of 70-130% were observed when performing spike and recovery experiments in saliva (Table 3). The ability of MOSAIC to detect IFN-γ levels in several saliva samples that were undetectable by Simoa despite being above its LOD may be attributed not only to its enhanced signal-to-background and sensitivity at low bead numbers, but also to higher sampling efficiencies that increase measurement precision at low concentrations. In addition, the more extensive washing performed in MOSAIC compared to Simoa, in order to minimize RCA products amplified from excess streptavidin-DNA, may contribute to improved removal of interfering components.

TABLE 3
Recoveries of spiked recombinant human IFN-γ and IL-
12p70 proteins for the MOSAIC assays performed in saliva
in three individual human saliva samples. Saliva samples
diluted four-fold in StartingBlock ™ Blocking
Buffer (Thermo Fisher Scientific). Recoveries are reported
as the mean ± standard deviation of duplicate measurements.

| | | | |
|---|---|---|---|
| Spiked Protein | Saliva #1 | Saliva #2 | Saliva #3 |

IFN-γ

| | | | |
|---|---|---|---|
| 0.2 fM | 84.6 ± 5.1% | 75.2 ± 11.2% | 70.7 ± 6.5% |
| 2 fM | 82.3 ± 6.5% | 78.1 ± 3.2% | 66.2 ± 17.9% |

IL-12p70

| | | | |
|---|---|---|---|
| 0.2 fM | 63.4 ± 0.0% | 40.2 ± 2.1% | 70.3 ± 8.1% |
| 2 fM | 78.2 ± 0.5% | 55.2 ± 2.6% | 73.3 ± 2.4% |

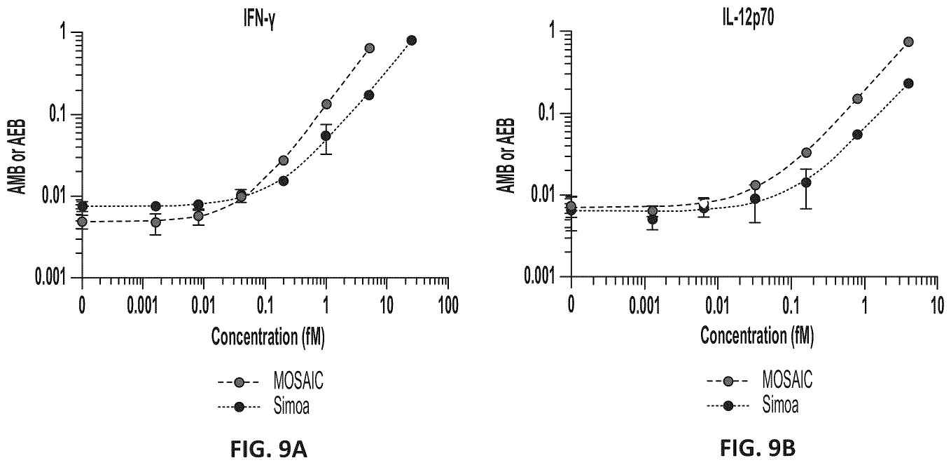

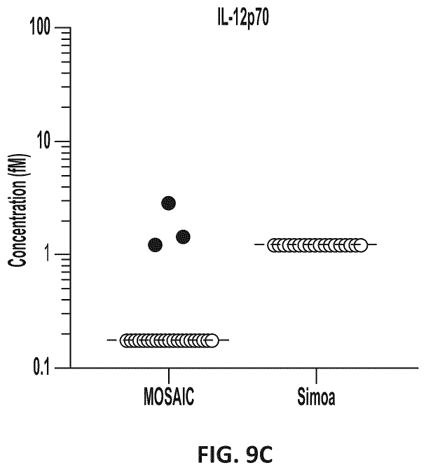

MOSAIC's ability to detect attomolar concentrations of endogenous proteins, which are undetectable by Simoa, highlights its potential diagnostic utility for very low abundance biomarkers. Using MOSAIC, improved detectability of an even lower-abundance analyte in saliva, IL-12p70, was also achieved from 0% by Simoa to approximately 12%, or 3/26 saliva samples (FIGS. 9A-9C). While the sensitivity of MOSAIC remained insufficient to detect IL-12p70 in the majority of the tested saliva samples, the results demonstrate that its enhanced sensitivity can begin to uncover the “tip of the iceberg” of such rare analytes in saliva but also underscore the need for even more sensitive methods.

### Multiplexing Capabilities of MOSAIC.

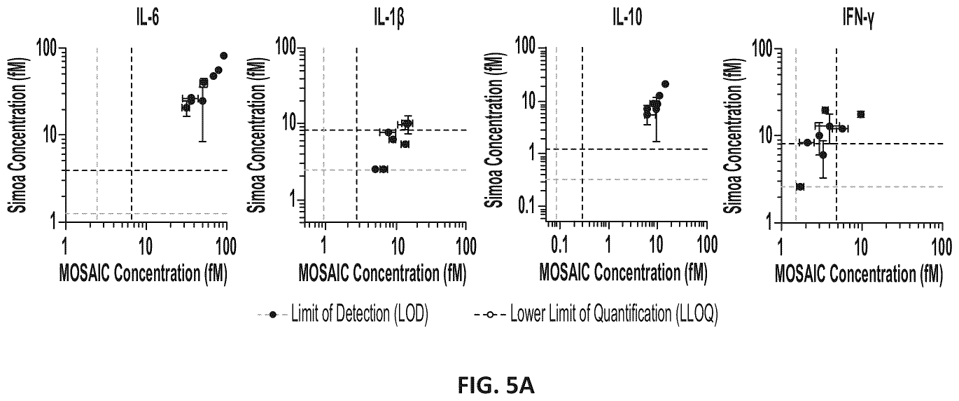

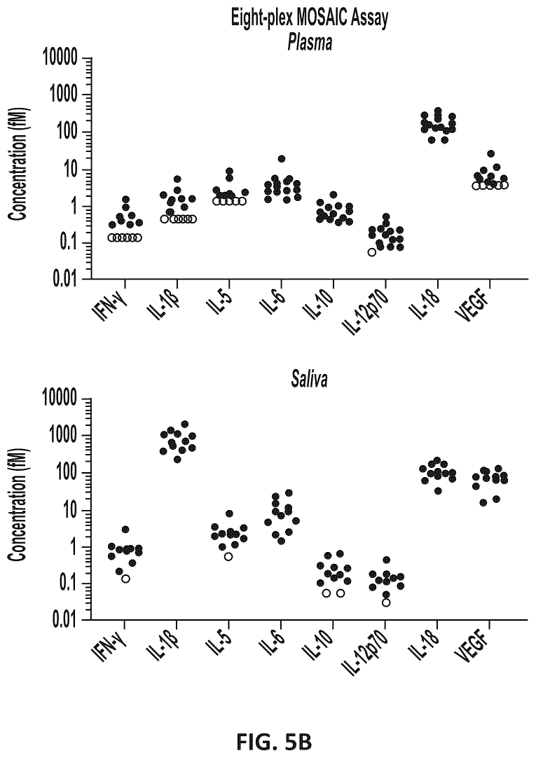

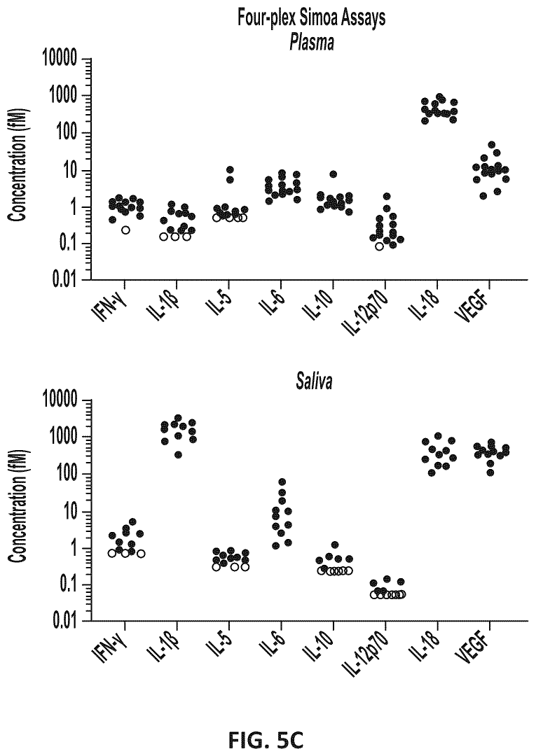

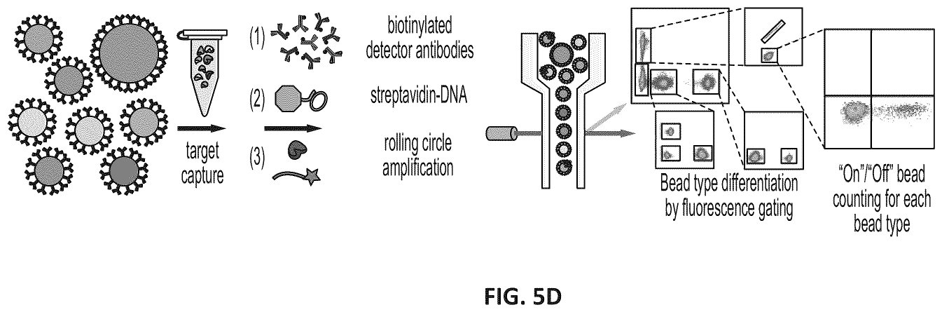

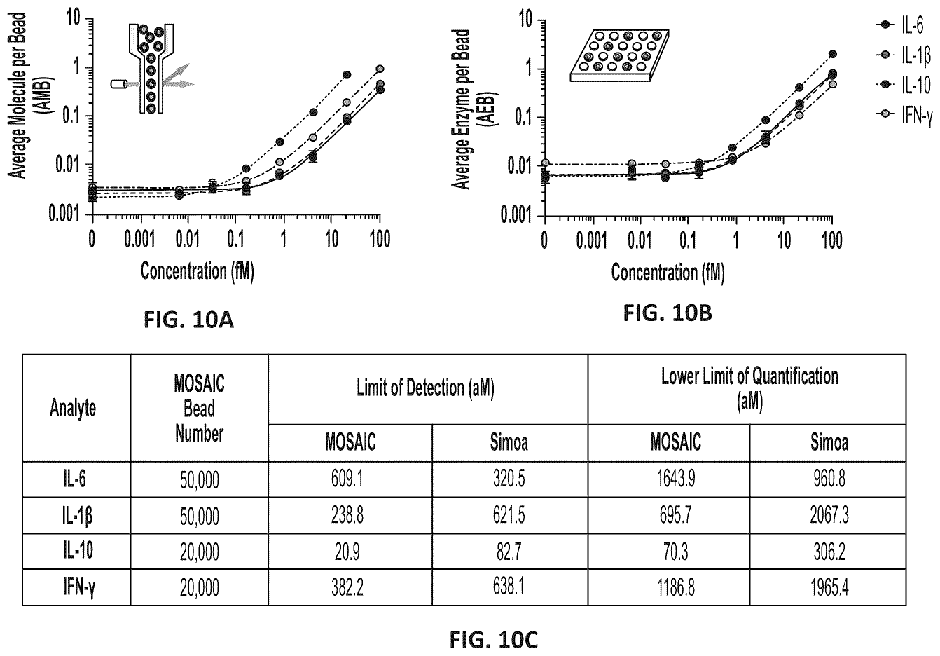

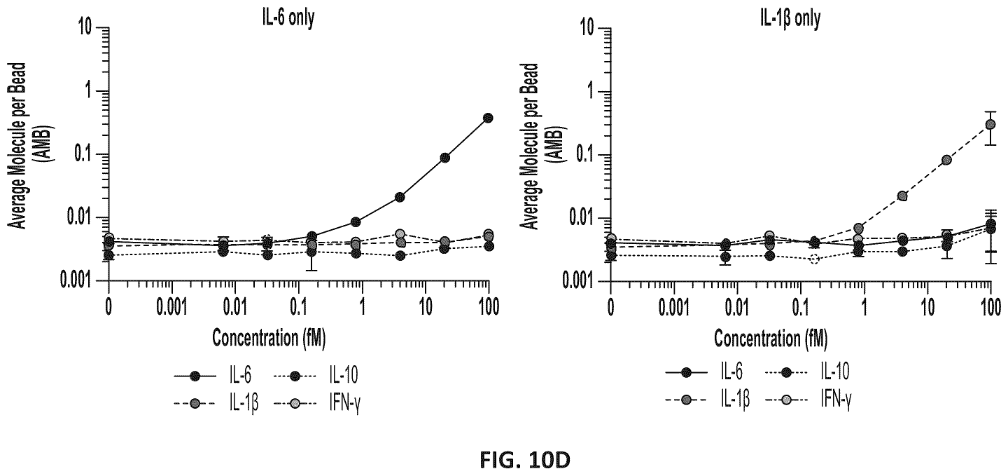

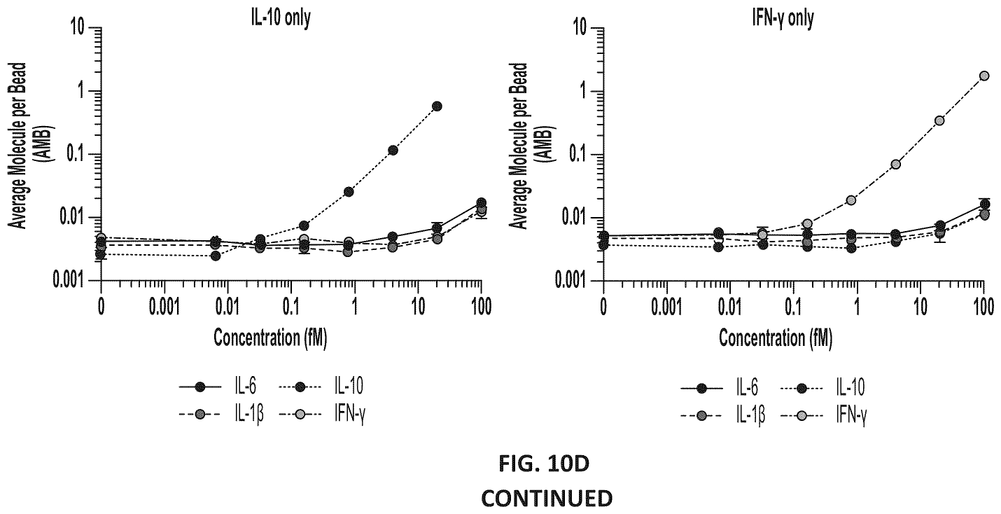

In addition to the enhanced sensitivity of MOSAIC, its on-bead signal generation strategy and flow cytometric readout can expand the multiplexing capabilities of digital ELISA. Current digital immunoassays are typically limited in the number of analytes that can be multiplexed, in part due to constraints in the total number of compartments for individual target isolation.¹⁵ In contrast, the solution-based readout of MOSAIC enables, in principle, an unlimited number of bead types to be analyzed in one sample with readily tunable sensitivities and dynamic ranges via assay bead number for each analyte. To verify the multiplexing capabilities of MOSAIC, we first developed a multiplexed assay for IL-6, IL-1β, IL-10, and IFN-γ, using beads encoded with different fluorescent dyes. This assay demonstrated mid- to high-attomolar sensitivities and acceptable recoveries in human plasma, with little cross-reactivity in the concentration ranges of the cytokines present in plasma (Table 4 and FIGS. 10A-10D). As further validation, its measurements in plasma correlated well with those of the corresponding four-plex Simoa assay, particularly for analyte concentrations well above the LLOQs of both methods (FIG. 5A).

TABLE 4
Recoveries of spiked recombinant proteins in for the four-plex MOSAIC assay
reported in FIG. 5 in human plasma at four-fold dilution. Recoveries are
reported as the mean ± standard deviation of duplicate measurements.

| Spiked | | | Spiked | | |
|---|---|---|---|---|---|
| Protein | Plasma #1 | Plasma #2 | Protein | Plasma #1 | Plasma #2 |
| IL-6 | | | IL-1β | | |
| 10 fM | 52.6 ± 19.0% | 82.0 ± 13.2% | 10 fM | 60.2 ± 6.0% | 71.8 ± 10.9% |
| 50 fM | 66.3 ± 0.3% | 74.9 ± 10.5% | 50 fM | 59.8 ± 0.8% | 54.6 ± 2.1% |
| IL-10 | | | IFN-γ | | |
| 1 fM | 85.4 ± 31.0% | 163.0 ± 22.9% | 10 fM | 101.8 ± 10.9% | 99.7 ± 8.3% |
| 10 fM | 90.2 ± 1.5% | 91.9 ± 9.5% | 50 fM | 105.9 ± 2.4% | 88.6 ± 0.6% |

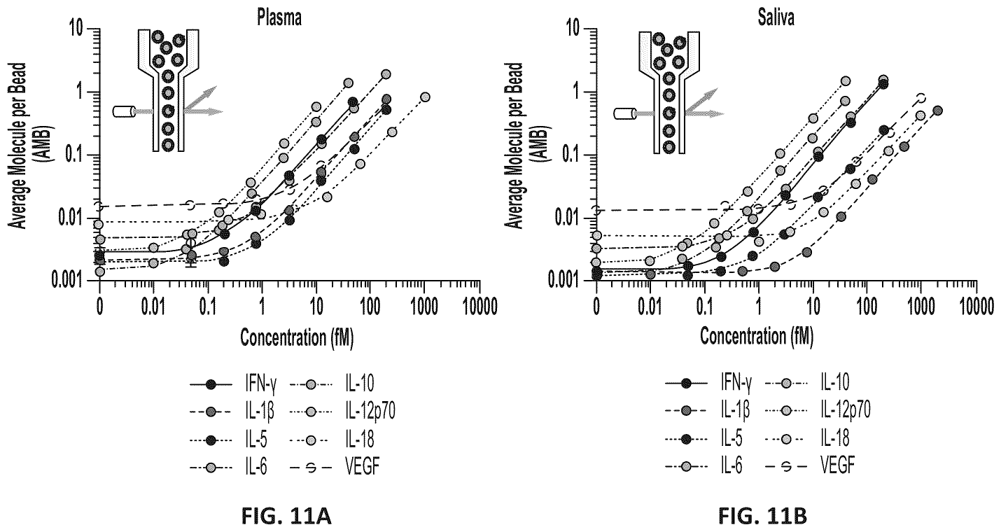

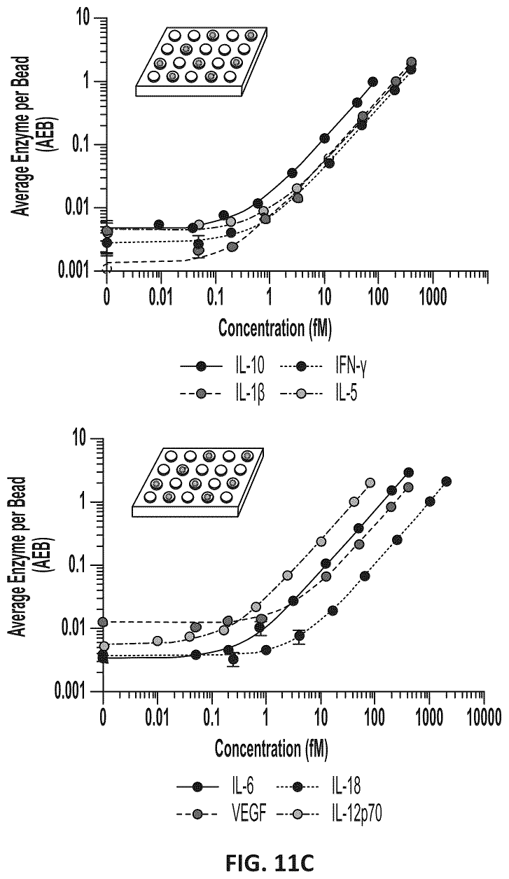

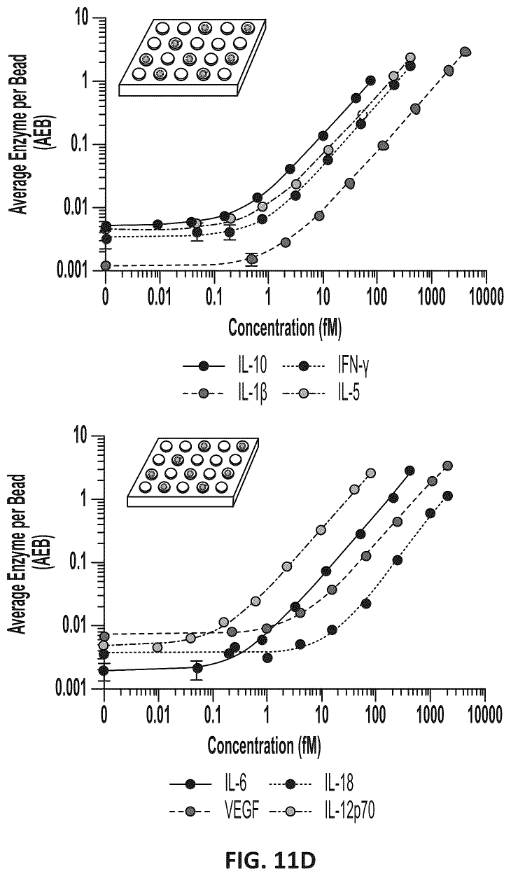

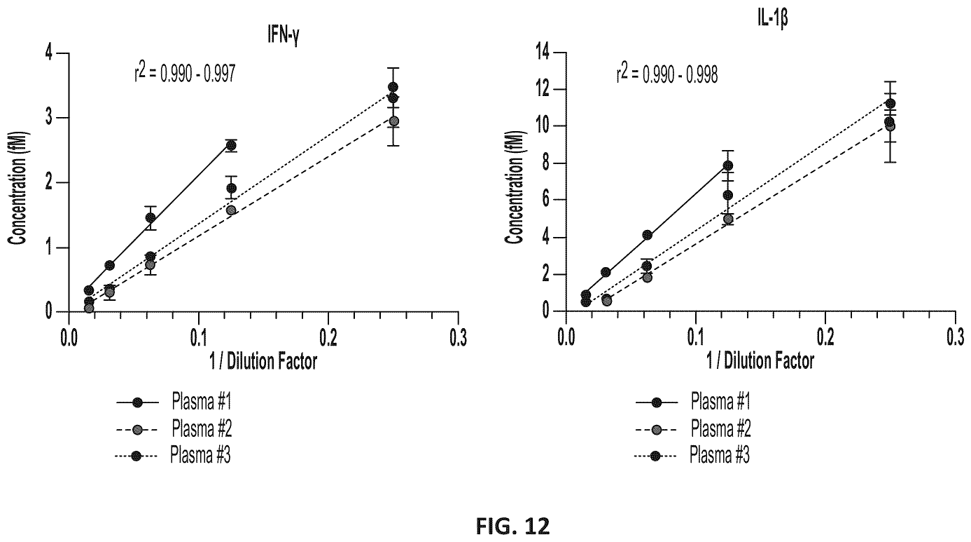

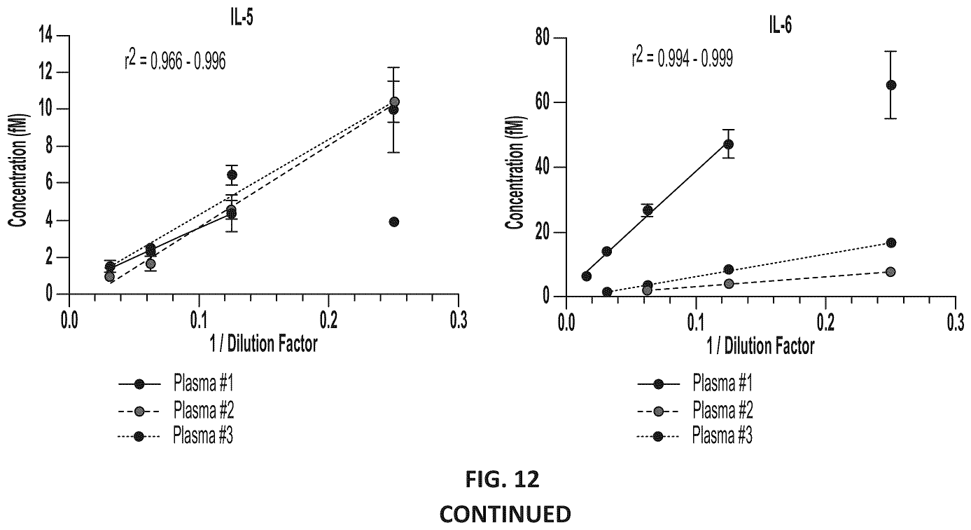

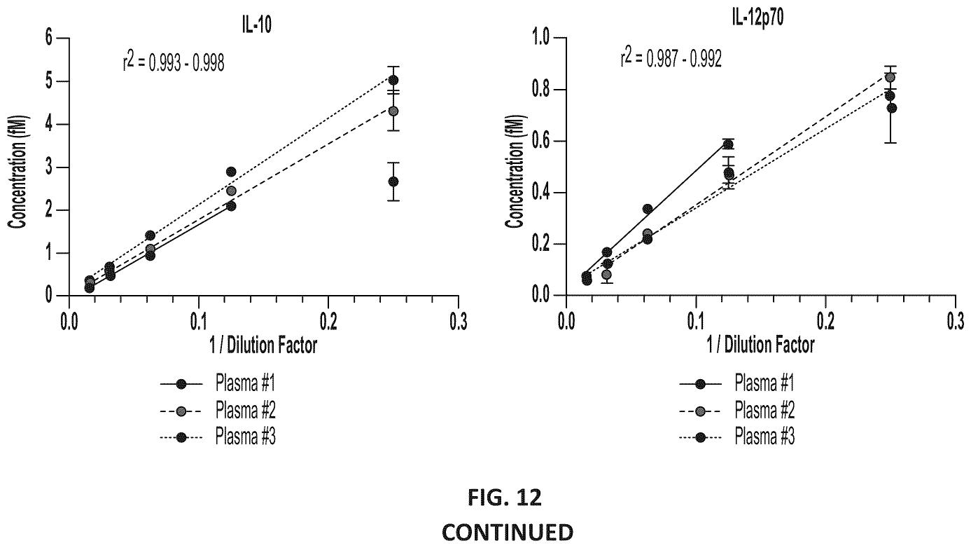

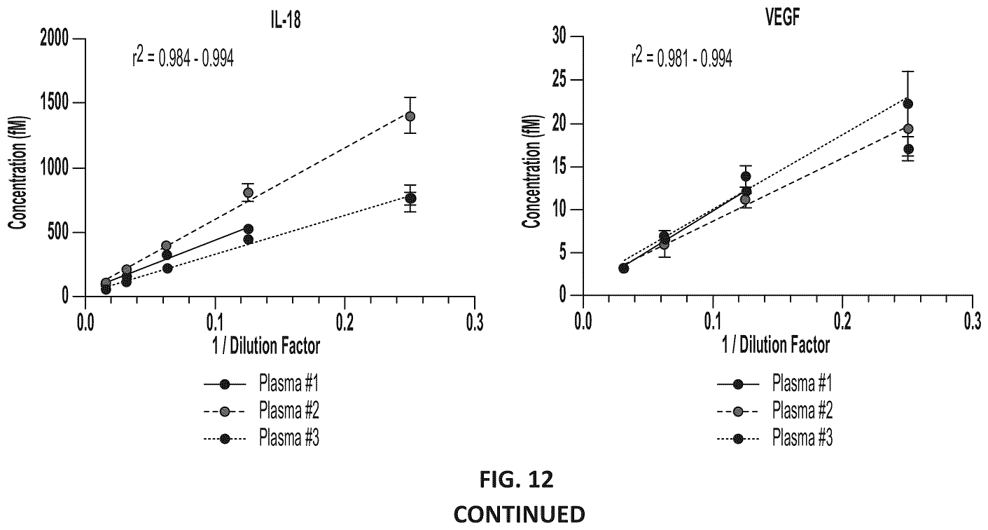

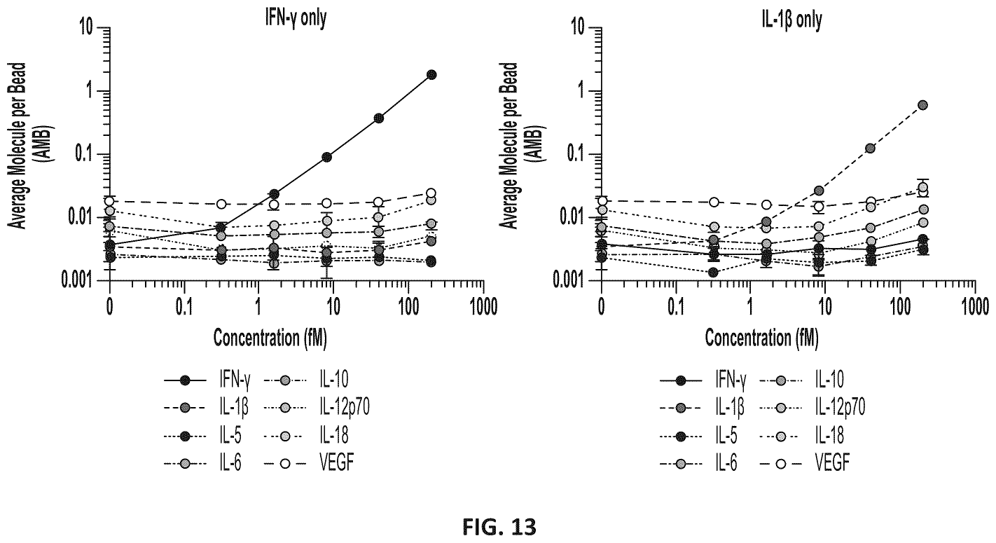

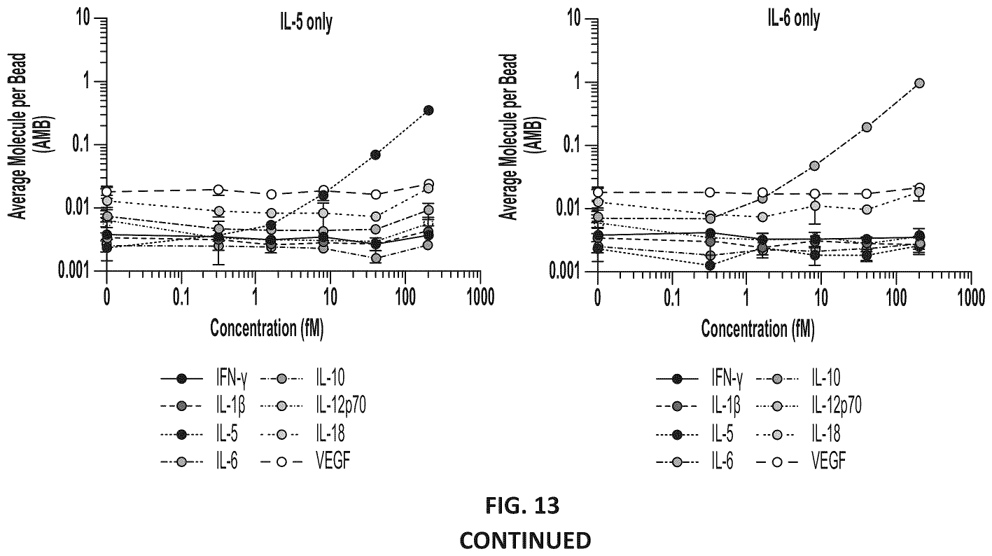

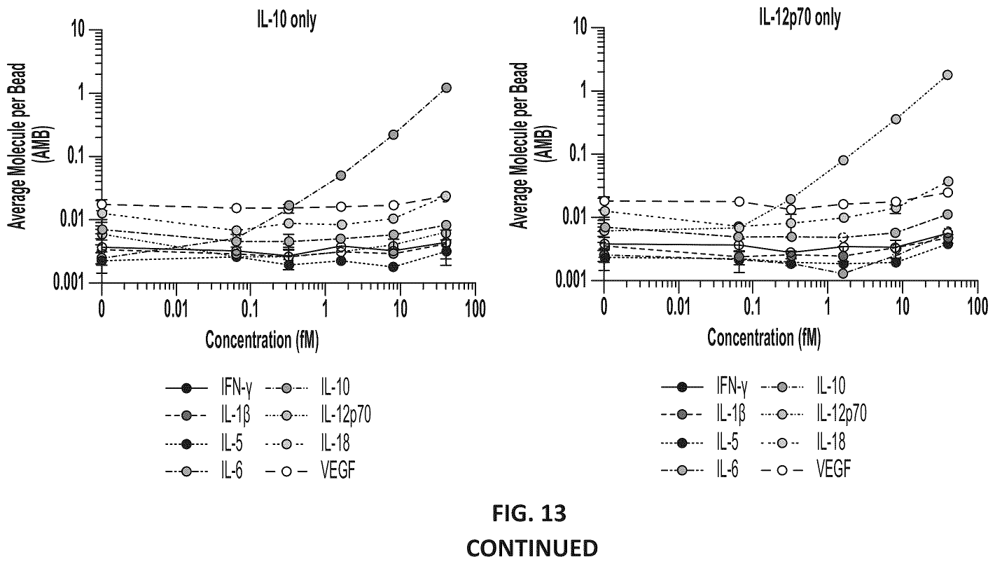

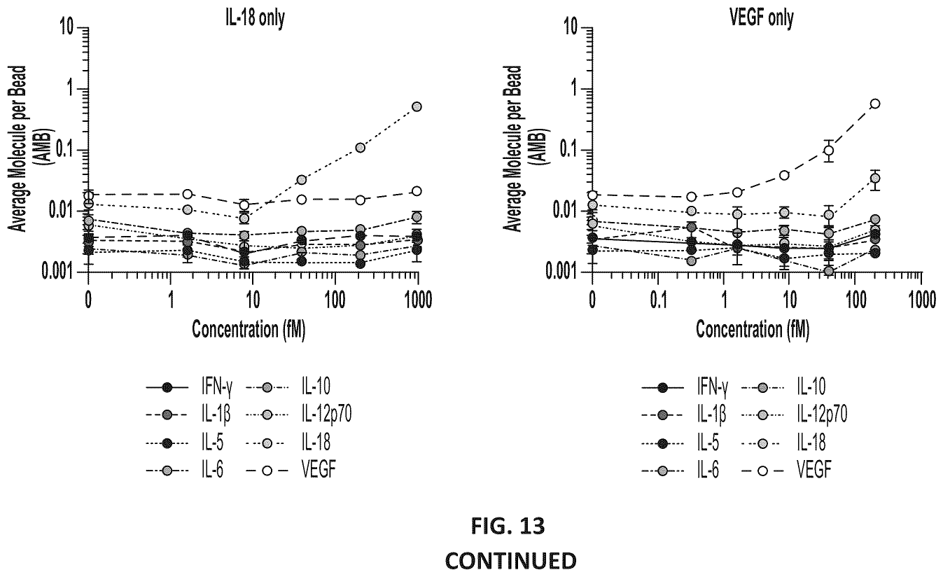

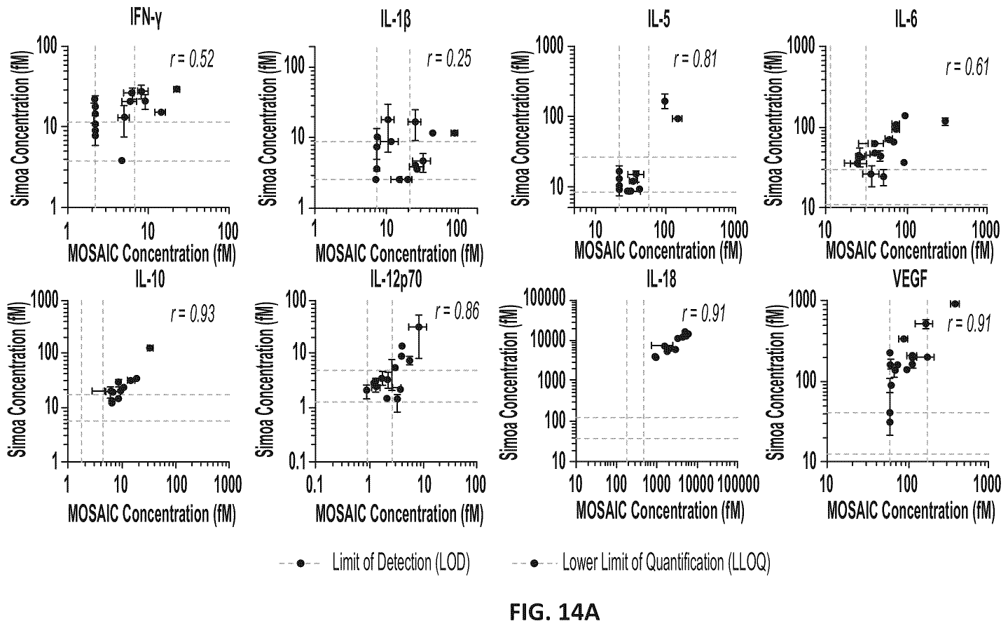

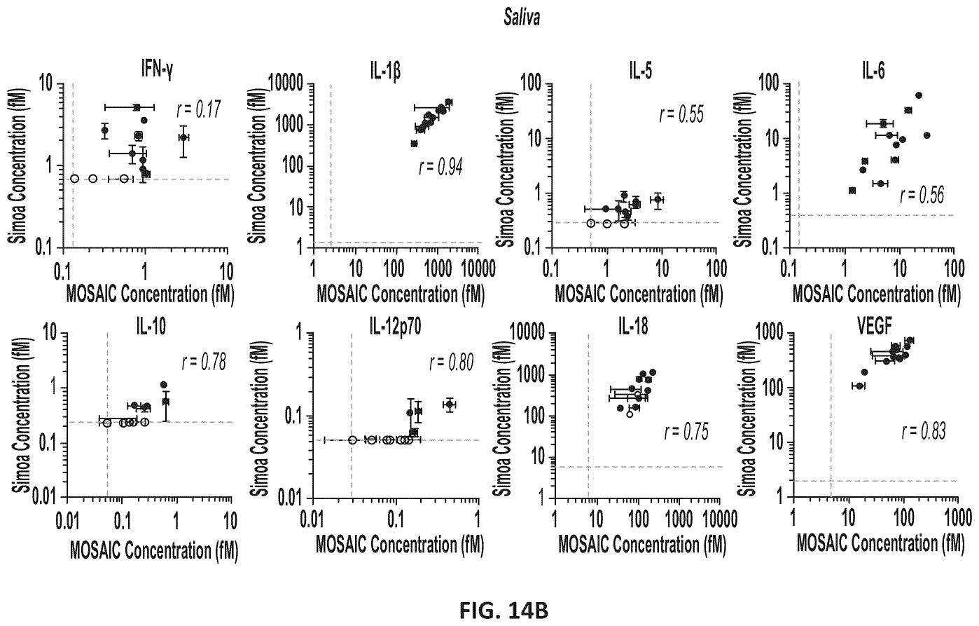

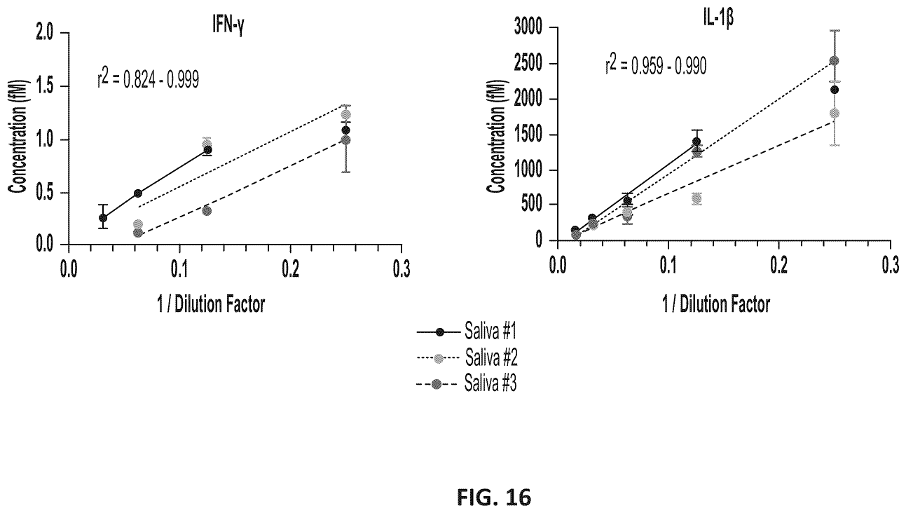

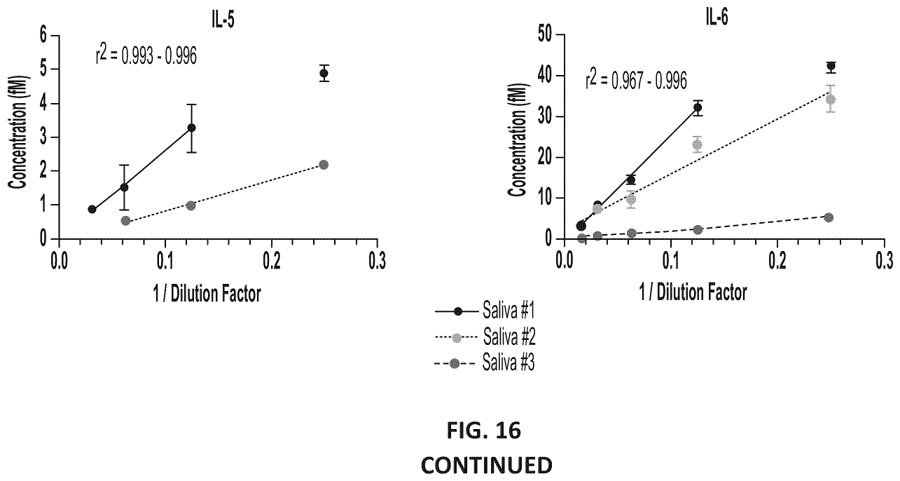

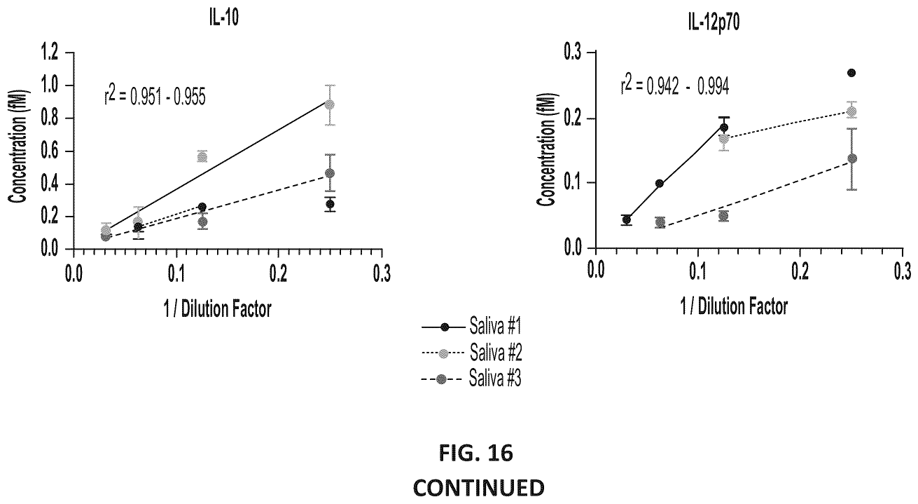

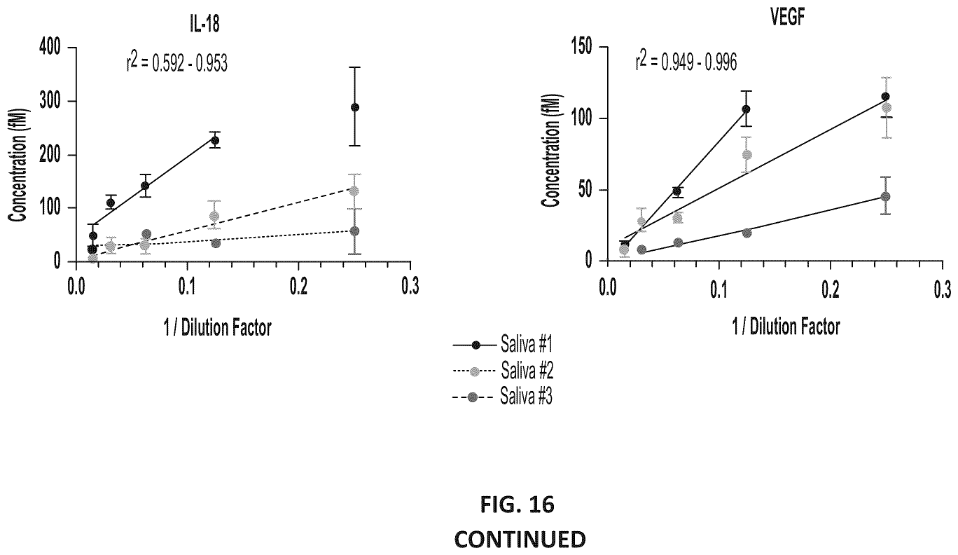

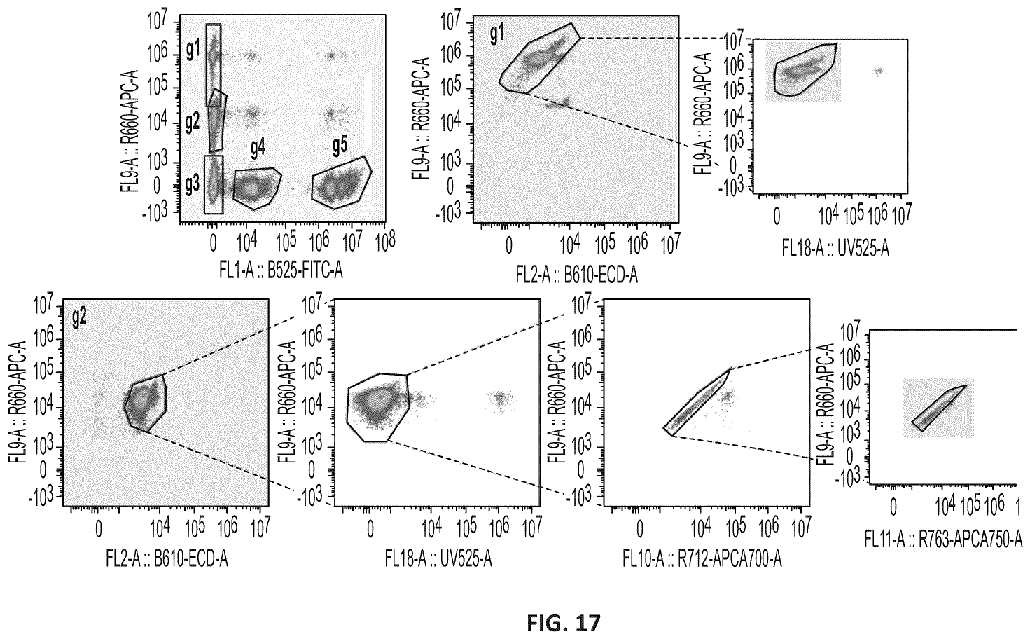

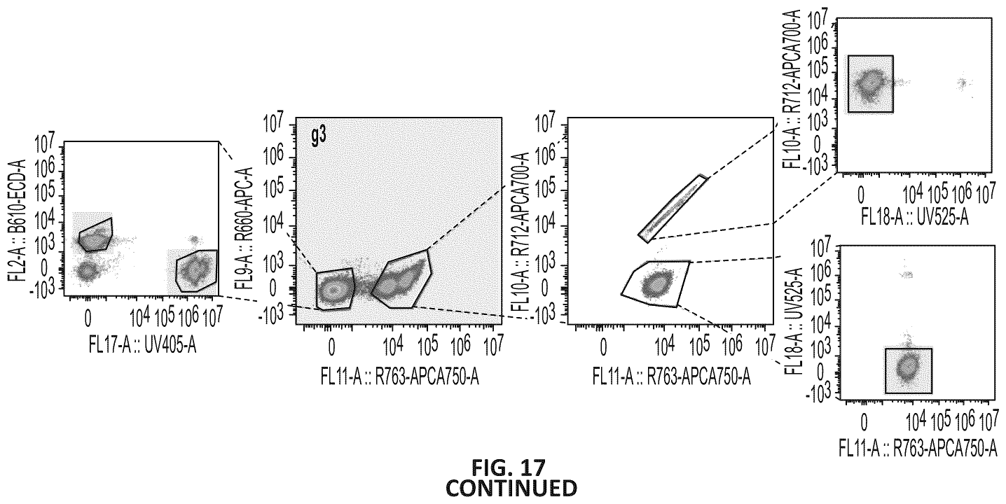

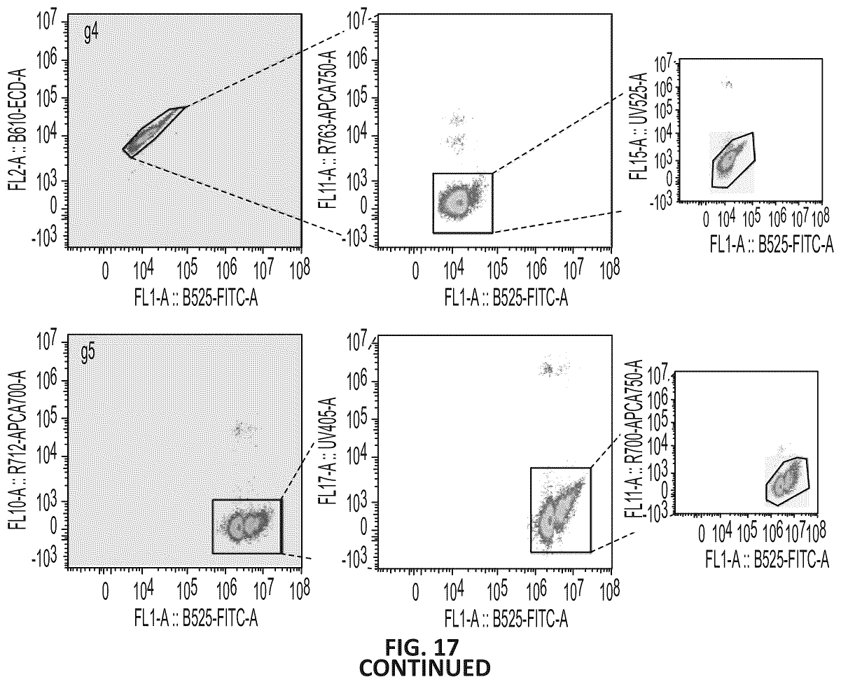

Having established the ability of MOSAIC to measure multiple analytes simultaneously with high accuracy, the increased multiplexing versatility of MOSAIC was then explored by incorporating additional beads with different fluorescent dyes and intensities. As a proof of principle, MOSAIC assays were integrated for the cytokines IL-6, IL-1β, IL-10, IFN-γ, IL-12p70, IL-5, and IL-18, and vascular endothelial growth factor (VEGF) into an eight-plex assay, which has not previously been reported with digital ELISA methods. High analytical sensitivities were maintained, with LOD values ranging from mid-attomolar to low femtomolar concentrations (FIGS. 11A-11D and Tables 5A-5B). The assay was applied to human plasma, using a 16-fold dilution factor to ensure acceptable recoveries across all analytes in spike and recovery experiments and consistent dilution linearity (Tables 6A, 6B and 7 and FIG. 12 and FIG. 16). Importantly, there was little cross-reactivity across the measured concentration ranges (FIG. 13). The exceptional sensitivity of MOSAIC further enables the use of higher dilution factors to minimize potential false signals due to cross-reactivity, in addition to allowing more clinical information to be collected from limited sample volumes. Using less than 15 μL of plasma across two replicates, these eight proteins were measured in a cohort of plasma samples and compared the measurements to those of two four-plex Simoa assays. The measured concentrations were generally correlated between the two methods for analytes with high detectability, further supporting the accuracy of the high-multiplex MOSAIC assay (FIGS. 5B-5C and FIGS. 14A-B). Thus, MOSAIC enables ultrasensitive multiplexed measurements of a broad biomarker panel using very small sample volumes.

TABLE 5A
Limit of detection (LOD) and lower limit of quantification (LLOQ) values of the
eight-plex MOSAIC assay and corresponding four-plex Simoa assays in the sample
diluent used for plasma measurements. Values are reported as the median [range]
of three calibration curves performed on separate days. LOD and LLOQ values are
calculated as three and ten standard deviations, respectively, above the background.

| | Limit of | | Lower Limit of | |
|---|---|---|---|---|
| | Detection (aM) | | Quantification (aM) | |
| Analyte | MOSAIC | Simoa | MOSAIC | Simoa |
| IFN-γ | 166.0 | 308.7 | 524.9 | 1,053.2 |
| | [136.6-194.4] | [240.3-717.6] | [434.3-614.3] | [714.4-2,192.7] |
| IL-1β | 445.5 | 322.1 | 1,357.8 | 976.3 |
| | [230.4-520.9] | [161.1-364.7] | [748.9-1,380.6] | [555.9-1,175.1] |
| IL-5 | 942.0 | 532.0 | 2,995.2 | 1,683.2 |
| | [539.4-1,373.3] | [352.1-1,139.5] | [1,523.4-3,470.6] | [1,175.6-3,920.6] |
| IL-6 | 707.2 | 426.7 | 1,919.7 | 1,350.7 |
| | [87.5-1,570.6] | [344.8-670.2] | [297.7-4,687.8] | [1,043.1-1,856.8] |
| IL-10 | 59.1 | 377.4 | 157.2 | 1,106.7 |
| | [33.2-107.2] | [216.4-381.0] | [106.3-287.1] | [682.4-1,257.3] |
| IL-12p70 | 54.6 | 83.6 | 163.1 | 312.0 |
| | [23.0-271.4] | [80.8-125.3] | [72.7-807.8] | [287.8-375.5] |
| IL-18 | 11,141.6 | 1,497.2 | 29,356.8 | 4,265.7 |
| | [4,922.8-37,997.9] | [1,319,0-2,460.8] | [16,539.1-114,204.2] | [3,958.0-7,678.9] |
| VEGF | 3,056.7 | 986.4 | 10,325.7 | 2,864.1 |
| | [1,965.9-3,673.4] | [766.5-1,163.7] | [6,963.4-10,831.0] | [2,583.8-3,002.8] |

TABLE 5B
Limit of detection (LOD) and lower limit of quantification (LLOQ)
values of the eight-plex MOSAIC assay and corresponding four-
plex Simoa assays in the sample diluent used for saliva measurements.
LOD and LLOQ values are calculated as three and ten standard
deviations, respectively, above the background.

| | |
|---|---|
| Limit of | Lower Limit of |
| Detection (aM) | Quantification (aM) |

| Analyte | MOSAIC | Simoa | MOSAIC | Simoa |
|---|---|---|---|---|
| IFN-γ | 131.1 | 698.4 | 386.6 | 2,204.4 |
| IL-1β | 2,537.1 | 1,381.0 | 7,711.1 | 4,307.7 |
| IL-5 | 524.8 | 282.2 | 1,640.4 | 999.4 |
| IL-6 | 157.1 | 388.2 | 507.9 | 1,163.6 |
| IL-10 | 53.8 | 238.9 | 154.9 | 765.8 |
| IL-12p70 | 29.9 | 52.4 | 88.8 | 160.8 |
| IL-18 | 6,288.5 | 5,863.5 | 16,242.8 | 16,201.3 |
| VEGF | 4,906.6 | 1,951.4 | 16,221.4 | 6,341.2 |

TABLE 6A
Recoveries of spiked recombinant human proteins for
the eight-plex MOSAIC assay in human plasma at
eight-fold dilution. Recoveries are reported
as the mean ± standard deviation of
duplicate measurements.

| | | | |
|---|---|---|---|
| Spiked Protein | Plasma #1 | Plasma #2 | Plasma #3 |

IFN-γ

| | | | |
|---|---|---|---|
| 2 fM | 80.3 ± 2.4% | 98.5 ± 2.4% | 82.8 ± 2.0% |
| 10 fM | 79.3 ± 1.0% | 76.8 ± 0.6% | 76.0 ± 3.8% |
| 50 fM | 67.8 ± 0.5% | 76.8 ± 9.0% | 71.3 ± 2.2% |

IL-1β

| | | | |
|---|---|---|---|
| 2 fM | 80.0 ± 1.8% | 124.2 ± 8.5% | 58.3 ± 11.4% |
| 10 fM | 67.4 ± 3.4% | 76.0 ± 1.0% | 58.8 ± 3.4% |
| 50 fM | 50.1 ± 3.9% | 64.3 ± 10.4% | 53.7 ± 5.4% |

IL-5

| | | | |
|---|---|---|---|
| 2 fM | 70.0 ± 5.5% | 58.3 ± 9.6% | 2.3 ± 0.3% |
| 10 fM | 66.1 ± 14.8% | 42.7 ± 3.9% | 19.1 ± 2.6% |
| 50 fM | 43.0 ± 6.0% | 43.2 ± 7.6% | 25.5 ± 0.7% |

IL-6

| | | | |
|---|---|---|---|
| 2 fM | 96.4 ± 37.0% | 180.7 ± 79.9% | 82.8 ± 15.2% |
| 10 fM | 87.5 ± 14.1% | 103.1 ± 14.3% | 83.6 ± 2.5% |
| 50 fM | 73.1 ± 0.6% | 82.8 ± 9.4% | 76.2 ± 4.2% |

IL-10

| | | | |
|---|---|---|---|
| 0.4 fM | 56.9 ± 43.0% | 137.0 ± 42.4% | 71.6 ± 6.5% |
| 2 fM | 78.9 ± 5.5% | 79.0 ± 8.8% | 73.0 ± 6.0% |
| 10 fM | 63.4 ± 6.7% | 68.4 ± 9.7% | 65.3 ± 4.0% |

IL-12p70

| | | | |
|---|---|---|---|
| 0.4 fM | 79.5 ± 13.1% | 81.7 ± 19.7% | 92.9 ± 21.6% |
| 2 fM | 75.4 ± 6.6% | 72.0 ± 4.1% | 75.7 ± 1.2% |
| 10 fM | 63.6 ± 0.1% | 70.4 ± 6.2% | 70.3 ± 0.0% |

IL-18

| | | | |
|---|---|---|---|
| 50 fM | 76.2 ± 7.3% | 121.7 ± 5.0% | 60.3 ± 8.7% |
| 250 fM | 31.3 ± 16.8% | 75.6 ± 1.6% | 54.5 ± 4.9% |

VEGF

| | | | |
|---|---|---|---|
| 10 fM | 70.7 ± 25.2% | 84.4 ± 10.2% | 61.8 ± 5.2% |
| 50 fM | 58.7 ± 8.4% | 81.7 ± 8.8% | 72.2 ± 0.1% |

TABLE 6B
Recoveries of spiked recombinant human proteins for
the eight-plex MOSAIC assay in human saliva at
eight-fold dilution. Recoveries are reported
as the mean ± standard deviation of
duplicate measurements.

| | | | |
|---|---|---|---|
| Spiked Protein | Saliva #1 | Saliva #2 | Saliva #3 |

IFN-γ

| | | | |
|---|---|---|---|
| 2 fM | 96.1 ± 5.0% | 113.7 ± 13.6% | 47.9 ± 16.6% |
| 10 fM | 70.6 ± 5.7% | 91.3 ± 7.4% | 29.3 ± 4.7% |
| 50 fM | 76.3 ± 6.3% | 83.5 ± 1.0% | 32.8 ± 10.9% |

IL-1β

| | | | |
|---|---|---|---|
| 20 fM | — | 57.3 ± 42.2% | — |
| 100 fM | — | 91.6 ± 17.0% | — |
| 500 fM | 112.0 ± 66.0% | 81.5 ± 4.6% | — |

IL-5

| | | | |
|---|---|---|---|
| 2 fM | 84.2 ± 38.4% | 68.4 ± 27.5% | 31.3 ± 10.3% |
| 10 fM | 51.6 ± 4.4% | 94.4 ± 2.2% | 13.2 ± 5.4% |
| 50 fM | 73.1 ± 12.7% | 79.7 ± 1.9% | 21.6 ± 13.3% |

IL-6

| | | | |
|---|---|---|---|
| 2 fM | — | 113.0 ± 9.7% | 90.5 ± 2.5% |
| 10 fM | 49.1 ± 12.1% | 99.3 ± 4.8% | 51.7 ± 21.9% |
| 50 fM | 83.3 ± 18.7% | 92.4 ± 3.2% | 57.0 ± 21.6% |

IL-10

| | | | |
|---|---|---|---|
| 0.4 fM | 91.2 ± 7.0% | 138.5 ± 35.8% | 60.1 ± 1.8% |
| 2 fM | 68.2 ± 4.0% | 93.8 ± 8.5% | 34.0 ± 8.0% |
| 10 fM | 77.4 ± 8.7% | 82.5 ± 1.6% | 37.7 ± 15.3% |

IL-12p70

| | | | |
|---|---|---|---|
| 0.4 fM | 85.5 ± 16.2% | 104.8 ± 16.0% | 34.5 ± 2.4% |
| 2 fM | 52.5 ± 0.1% | 83.4 ± 1.8% | 22.6 ± 5.2% |
| 10 fM | 58.4 ± 4.8% | 82.1 ± 1.9% | 24.5 ± 9.9% |

IL-18

| | | | |
|---|---|---|---|
| 50 fM | — | 79.7 ± 37.9% | — |
| 250 fM | 37.1 ± 36.1% | 60.3 ± 2.1% | 17.6 ± 23.7% |

VEGF

| | | | |
|---|---|---|---|
| 50 fM | 29.0 ± 20.1% | 101.2 ± 7.7% | 22.2 ± 25.0% |
| 250 fM | 66.4 ± 20.5% | 91.5 ± 4.4% | 39.5 ± 24.1% |

TABLE 7
Recoveries of spiked recombinant human proteins for
the eight-plex MOSAIC assay in human plasma at 16-
fold dilution. Recoveries are reported as the
mean ± standard deviation of duplicate
measurements.

| | | | |
|---|---|---|---|
| Spiked Protein | Plasma #1 | Plasma #2 | Plasma #3 |

IFN-γ

| | | | |
|---|---|---|---|
| 2 fM | 86.5 ± 1.9% | 90.1 ± 2.8% | 84.6 ± 4.2% |
| 10 fM | 69.5 ± 8.1% | 67.4 ± 4.3% | 72.2 ± 7.3% |
| 50 fM | 78.4 ± 1.5% | 72.7 ± 1.5% | 79.1 ± 3.0% |

IL-1β

| | | | |
|---|---|---|---|
| 2 fM | 88.0 ± 15.7% | 80.4 ± 28.8% | 79.5 ± 7.4% |
| 10 fM | 55.3 ± 8.0% | 56.5 ± 6.8% | 61.3 ± 2.0% |
| 50 fM | 60.0 ± 1.9% | 55.2 ± 2.1% | 61.2 ± 1.5% |

IL-5

| | | | |
|---|---|---|---|
| 2 fM | 102.4 ± 3.9% | 202.8 ± 52.8% | 145.1 ± 15.3% |
| 10 fM | 55.8 ± 4.0% | 79.8 ± 40.8% | 84.1 ± 11.4% |
| 50 fM | 72.1 ± 2.8% | 73.3 ± 0.1% | 72.0 ± 1.3% |

IL-6

| | | | |
|---|---|---|---|
| 2 fM | 105.8 ± 20.8% | 112.3 ± 16.4% | 96.2 ± 3.4% |
| 10 fM | 74.2 ± 6.5% | 77.3 ± 0.8% | 77.9 ± 2.4% |
| 50 fM | 82.1 ± 2.8% | 78.9 ± 0.2% | 78.3 ± 0.6% |

IL-10

| | | | |
|---|---|---|---|
| 0.4 fM | 77.8 ± 9.4% | 81.4 ± 3.5% | 83.1 ± 26.0% |
| 2 fM | 54.0 ± 7.9% | 57.3 ± 14.5% | 53.4 ± 6.5% |
| 10 fM | 68.8 ± 9.3% | 55.3 ± 3.6% | 53.7 ± 12.5% |

IL-12p70

| | | | |
|---|---|---|---|
| 0.4 fM | 112.1 ± 7.9% | 97.3 ± 2.5% | 88.7 ± 11.2% |
| 2 fM | 69.4 ± 3.6% | 68.3 ± 3.9% | 74.0 ± 4.6% |
| 10 fM | 77.5 ± 0.9% | 67.7 ± 0.4% | 73.4 ± 0.5% |

IL-18

| | | | |
|---|---|---|---|
| 50 fM | — | 49.9 ± 55.9% | 72.7 ± 25.7% |
| 250 fM | 48.7 ± 13.6% | 60.3 ± 6.8% | 61.7 ± 6.6% |

VEGF

| | | | |
|---|---|---|---|
| 10 fM | 48.7 ± 20.1% | 88.2 ± 1.1% | 91.7 ± 10.1% |
| 50 fM | 85.7 ± 1.1% | 81.0 ± 4.1% | 84.7 ± 5.9% |

### Discussion

The ability to measure very low levels of protein biomarkers remains a critical challenge in clinical applications such as cancer, infectious disease, and neurodegenerative disease diagnostics. While digital measurement technologies have enabled 1000-fold increases in sensitivity, proteins at attomolar or lower concentrations remain inaccessible, limiting efforts in biomarker discovery and early detection of diseases in which biomarker levels may be very low during initial stages. One notable example is saliva, which is collected non-invasively and contains diverse proteins of potential diagnostic value but at much lower levels than in plasma, representing an exceptional challenge to existing digital ELISA technologies. The proof-of-principle measurements of IFN-γ and IL-12p70 in saliva highlight the potential utility of MOSAIC in unveiling uncharted territories of rare biomarker candidates. Importantly, because of the high sampling efficiencies of MOSAIC, the inventors were able to reduce the number of assay beads to enhance the signal-to-background while maintaining sufficient measurement precision. While reducing bead numbers improved sensitivity to differing extents for different analytes, the results show that bead number can be readily tuned for each analyte according to the desired sensitivity and dynamic range required for the specific application.

Importantly, MOSAIC circumvents the requirement for specialized instrumentation, which has restricted widespread implementation of digital ELISA technologies. By eliminating the need to isolate beads into individual compartments, MOSAIC enables single molecule counting using flow cytometry, thus translating digital ELISA to existing, widely available laboratory infrastructure. A key advantage of MOSAIC over previous digital ELISA methods, including the recently developed dropcast single molecule assays and droplet digital ELISA, is the much faster, automated signal readout-less than one minute per sample. The dropcast single molecule assays (dSimoa) enabled a simple bead dropcasting method for counting single molecules captured on the beads, increasing sampling efficiencies and analytical sensitivity by approximately tenfold. However, these methods require long imaging times per sample to capture all the beads across multiple fields of view, thus limiting throughput. Automation via the 96-well plate sampling modes that are already built into many benchtop flow cytometers further provides a streamlined workflow.

In addition to enhanced sensitivity and an accessible workflow, MOSAIC introduces increased multiplexing capabilities to digital ELISA. The ability to simultaneously measure multiple analytes in a single sample can accelerate sample throughput and biomarker signature discovery and is especially critical in applications with limited sample volumes, such as neonatal saliva or fingerprick blood. Multiplexing in digital ELISA as well as in a recently developed ultrasensitive planar ELISA platform has shown great utility in various diagnostic applications¹⁶⁻¹⁸, but the number of bead types that can be analyzed simultaneously in bead confinement methods is limited by the total number of compartments and low bead analysis efficiencies. In contrast, the solution-based readout of MOSAIC eliminates this physical constraint on the total number of bead types that can be analyzed in a single sample. As sampling efficiencies in MOSAIC are unaffected by the total number of beads, the desired dynamic range for each analyte in a multiplex MOSAIC assay is readily tunable by the assay bead number, with no upper limit on the number of beads or bead types in each sample. In addition to multiplexing by color and fluorescence intensity, the versatile flow cytometric readout provides the ability to distinguish beads by size, thus expanding the palette of multiplexable bead types. While cross-reactivity remains a potential limitation in higher order multiplex assays, the attomolar sensitivity of MOSAIC can enable higher dilution factors to be used while maintaining good target detectability, thus keeping measurements in concentration ranges where cross-reactivity is minimal. In addition, smaller multiplex panels may be used with subsequent combination of all beads into a single sample for signal readout, as well as sequential target capture.¹⁹

Despite the low attomolar sensitivities of MOSAIC, further work is required to achieve even higher sensitivity and detectability for rare analytes. With the development and screening of higher-affinity reagents, sub-attomolar LODs may be attained. Furthermore, while washing steps in the current MOSAIC workflow are automated using a microplate washer, future work will explore integration of the entire assay into an automated liquid handling platform for improved measurement precision.

In summary, by leveraging the power of on-bead signal amplification and the multifaceted capabilities of flow cytometry, MOSAIC provides an ultrasensitive protein detection method with attomolar sensitivity, higher-order multiplexing, and rapid high-throughput readout, accessible to any lab with a flow cytometer.

### Methods

### Materials

All affinity reagents, recombinant proteins, and DNA oligos used in this work are listed below. Buffers and paramagnetic beads were purchased from Quanterix Corporation and Bangs Laboratories. Custom DNA oligos were purchased from Integrated DNA Technologies and MilliporeSigma.

| | Capture | Detector | Recombinant |
|---|---|---|---|
| Analyte | Antibody | Antibody | Protein |
| IL-10 | 506802 | 501501 | 217-IL-005 |
| | (Biolegend) | (Biolegend) | (R&D Systems) |
| IFN-γ | 507502 | MAB285 | 285-IF-100 |
| | (Biolegend) | (R&D Systems) | (R&D Systems) |
| IL-6 | MAB206 | BAF206 | 206-IL-010 |
| | (R&D Systems) | (R&D Systems) | (R&D Systems) |
| IL-1β | 508202 | 511704 | 201-LB-005 |
| | (Biolegend) | (Biolegend) | (R&D Systems) |
| IL-8 | 554716 | 554718 | 208-IL-010 |
| | (BD Biosciences) | (BD Biosciences) | (R&D Systems) |
| IL-12p70 | 511002 | 508801 | 219-IL-005 |
| | (Biolegend) | (Biolegend) | (R&D Systems) |
| IL-5 | 500902 | 501002 | 205-IL-005 |
| | (Biolegend) | (Biolegend) | (R&D Systems) |
| IL-18 | D044-3 | D045-6 | B003-5 |
| | (R&D Systems) | (R&D Systems) | (R&D Systems) |
| VEGF | M808 | BAF293 | DY293B-05 |
| | (Thermo Fisher Scientific) | (R&D Systems) | (R&D Systems) |

| | |
|---|---|
| Primer | 5′-Azide- TTTTTTTTTTTTTTTTAGACACCGTTCCT |
| | TGGACAGA*G*C (SEQ ID NO: 1) |
| Template | 5′-Phosphate- GAACGGTGTCTATTATGTCCTATCC |
| | TCAGCTATTATGTCCTATCCTCAGCTATTATGTCCTATC |
| | CTCAGCTCTGTCCAAG (SEQ ID NO: 2) |
| Probe | 5′-[ATTO 647N or ATTO 565]- TATTATGTCCT |
| | ATCCTCAGC -InvdT (SEQ ID NO: 3) |

### Preparation of Capture and Labeling Reagents.

Capture antibodies were buffer exchanged with Bead Conjugation Buffer (Quanterix) using a 50K Amicon Ultra-0.5 mL centrifugal filter (MilliporeSigma). After adding Bead Conjugation Buffer to antibody solution in the filter up to 500 μL, buffer exchange was carried out by centrifuging three times at 14,000×g for five minutes, with addition of 450 μL Bead Conjugation Buffer between centrifugation cycles. The buffer-exchanged antibody was recovered by inverting the filter into a new tube, centrifuging at 1000×g for two minutes, rinsing the filter with 50 μL Bead Conjugation Buffer, and centrifuging one more time at 1000×g for two minutes. The concentration of the buffer-exchanged antibody was then measured using a NanoDrop spectrophotometer. For each bead type, beads were washed three times with 300 μL Bead Wash Buffer (Quanterix) and two times with 300 μL Bead Conjugation Buffer (Quanterix) before resuspending in cold Bead Conjugation Buffer. Bead number and conjugation conditions for each analyte are shown in table below. A 1 mg vial of 1-ethyl-3-(3-dimethylaminopropyl) carbodiimide hydrochloride (EDC) (Thermo Fisher Scientific) was dissolved in 100 μL cold Bead Conjugation Buffer, and the desired volume was added to the beads. The beads were shaken for 30 minutes at either room temperature or 4° C. After EDC activation of the carboxyl groups on the beads, the beads were washed once with 300 μL cold Bead Conjugation Buffer before resuspension in the buffer-exchanged antibody solution. Antibody conjugation was carried out by shaking the beads for two hours at either room temperature or 4° C., followed by washing twice with 300 μL Bead Wash Buffer. The antibody-coupled beads were then blocked for thirty minutes at room temperature with shaking in 300 μL Bead Blocking Buffer (Quanterix). After washing once each with 300 μL Bead Wash Buffer and Bead Diluent (Quanterix), the beads were resuspended in Bead Diluent, counted with a Beckman Coulter Z1 Particle Counter, and stored at 4° C.

TABLE 8
Coupling conditions for the antibody-coated capture beads used in this
Example. For comparison of the eight-plex MOSAIC assay to Simoa, a four-
plex assay for IL-6, VEGF, IL-18, and IL-12p70 using 488, 647, 700, and
750 multiplex beads, respectively, from Quanterix, was performed.

| | | | | | | |
|---|---|---|---|---|---|---|
| | | | Starting | | | Capture |
| | | | Bead | Coupling | EDC | Antibody |
| Analyte | Bead Type | Bead Vendor | Number | Temperature | (μL) | (μg) |

Single-plex Assays

| | | | | | | |
|---|---|---|---|---|---|---|
| IL-10 | 488 multiplex | Quanterix | 2.8 × 10⁸ | room | 10 | 60 |
| | beads | | | temperature | | |
| IFN-γ | 488 multiplex | | 4.2 × 10⁸ | 4° C. | 9 | 60 |
| | beads | | | | | |
| IL-6 | 488 multiplex | | 2.8 × 10⁸ | room | 10 | 100 |
| | beads | | | temperature | | |
| IL-1β | 488 multiplex | | 4.2 × 10⁸ | 4° C. | 9 | 60 |
| | beads | | | | | |
| IL-8 | 700 multiplex | | 2.8 × 10⁸ | room | 6 | 80 |
| | beads | | | temperature | | |
| IL-12p70 | Singleplex beads | | 4.2 × 10⁸ | 4° C. | 9 | 60 |

Four-plex Assay

| | | | | | | |
|---|---|---|---|---|---|---|
| IL-6 | 488 multiplex | Quanterix | 2.8 × 10⁸ | room | 10 | 100 |
| | beads | | | temperature | | |
| IL-1β | 647 multiplex | | 4.2 × 10⁸ | 4° C. | 9 | 60 |
| | beads | | | | | |
| IL-10 | 700 multiplex | | 2.8 × 10⁸ | room | 5 | 60 |
| | beads | | | temperature | | |
| IFN-γ | 750 multiplex | | 4.2 × 10⁸ | 4° C. | 9 | 60 |
| | beads | | | | | |

Eight-plex Assay

| | | | | | | |
|---|---|---|---|---|---|---|
| IFN-γ | 700 multiplex | Quanterix | 4.2 × 10⁸ | 4° C. | 9 | 60 |
| | beads | | | | | |
| IL-1β | 647 multiplex | Quanterix | 4.2 × 10⁸ | 4° C. | 9 | 60 |
| | beads | | | | | |
| IL-5 | 750 multiplex | Quanterix | 4.2 × 10⁸ | 4° C. | 9 | 60 |
| | beads | | | | | |
| IL-6 | COMPEL ™ | Bangs | 2.8 × 10⁸ | room | 10 | 100 |
| | COOH-modified | Laboratories | | temperature | | |
| | Glacial Blue | | | | | |
| | beads, 3 μm | | | | | |
| IL-10 | 488 multiplex | Quanterix | 2.8 × 10⁸ | room | 10 | 60 |
| | beads | | | temperature | | |
| IL-12p70 | Singleplex beads | Quanterix | 4.2 × 10⁸ | 4° C. | 9 | 60 |
| IL-18 | QuantumPlex ™ | Bangs | 1.0 × 10⁸ | 4° C. | 15 | 2 |
| | M SP COOH | Laboratories | | | | |
| VEGF | COMPEL ™ | Bangs | 4.2 × 10⁸ | 4° C. | 5 | 60 |
| | COOH-modified | Laboratories | | | | |
| | Dragon Green | | | | | |
| | beads, 3 μm | | | | | |

Detector antibodies were obtained in biotinylated form except for the IFN-γ detector antibody, which was obtained in unmodified form. The IFN-γ detector antibody was biotinylated by reconstituting to 1 mg/mL in Biotinylation Reaction Buffer (Quanterix) and adding a 40-fold molar excess of NHS-PEG4-Biotin (Thermo Fisher Scientific) freshly dissolved in water. The biotinylation reaction was carried out for thirty minutes at room temperature, followed by purification of the biotinylated antibody with a 50K Amicon Ultra-0.5 mL centrifugal filter. Five centrifugation cycles of 14,000×g for five minutes with addition of 450 μL Biotinylation Reaction Buffer between cycles were performed, with subsequent recovery of the purified antibody via inversion of the filter into a new tube and centrifuging at 1000×g for two minutes. The filter was then rinsed with 50 μL Biotinylation Reaction Buffer before centrifuging one more time at 1000×g for two minutes and quantifying the antibody concentration with a NanoDrop spectrophotometer.

### Preparation of Streptavidin-DNA Conjugate.

A 5′ azide-modified primer was annealed to the DNA template for RCA by heating a solution of 33.8 μM primer and 40.6 μM template in NEBNext Quick Ligation Buffer (New England Biolabs) at 95° C. for two minutes and allowing to cool to room temperature over 90 minutes. Ligation was then performed with addition of T4 DNA ligase and incubation at room temperature for three hours. The ligation reaction was then heated at 65° C. for 10 minutes to inactivate the ligase, with subsequent cooling to room temperature. The ligation reaction was buffer exchanged into phosphate buffered saline (PBS) with 1 mM EDTA using a 7K MWCO Zeba spin desalting column (Thermo Fisher Scientific). For conjugation, streptavidin (Biolegend 280302) was buffer exchanged into PBS using a 10K Amicon Ultra-0.5 mL centrifugal filter, incubated with a 20-fold molar excess of dibenzocyclooctyne-PEG4-N-hydroxysuccinimidyl ester (DBCO-PEG4-NHS, MilliporeSigma) for 30 minutes at room temperature, and purified with a 10K Amicon Ultra-0.5 mL centrifugal filter in PBS with 1 mM EDTA. A two-fold molar excess of the ligated primer-template was then added to the DBCO-modified streptavidin and incubated overnight at 4° C. The conjugate was stored in aliquots at −80° C. in PBS with 5 mM EDTA, 0.1% BSA, and 0.02% sodium azide.

### Mosaic Assays.

MOSAIC assays were performed in a 96-well plate (Greiner Bio-One, 655096), with antibody-coated beads and detector antibodies diluted to the desired concentrations in Homebrew Sample Diluent (Quanterix). Sample volumes of 100 μL were used, with 10 μL of antibody-coated beads. For two-step assays, 10 μL detector antibody was added to each sample. The plate was sealed and shaken for one hour for target capture, followed by washing with System Wash Buffer 1 (Quanterix) using a BioTek 405 TS Microplate Washer. For three-step assays, 100 μL detector antibody was added to the beads after target capture and washing steps, followed by a 10-minute incubation and additional washing. The immunocomplex sandwiches were then labeled with streptavidin-DNA by adding 100 μL of the conjugate diluted in Sample Diluent with 5 mM EDTA to the beads and shaking for the desired time. The samples were then washed with System Wash Buffer 1 for eight cycles, transferred to a new 96-well plate, and washed an additional time with 200 μL System Wash Buffer 1 before being resuspended in 60 μL of the RCA reaction mixture. The RCA mixture consisted of 0.5 mM deoxynucleotide mix (New England Biolabs), 0.33 U/uL phi29 DNA polymerase (Lucigen), 0.2 mg/mL bovine serum albumin (BSA, New England Biolabs), 1 nM fluorescently labeled DNA probe (Integrated DNA Technologies), and 0.1% Tween-20 in 50 mM Tris-HCl (pH 7.5), 10 mM (NH₄)₂SO₄, and 10 mM MgCl₂. ATTO-647N and ATTO-565 labeled DNA probes were used for single target and multiplex MOSAIC assays, respectively. Upon addition of the RCA mixture to each sample, the plate was shaken for one hour at 37° C., followed by addition of 150 μL PBS with 0.1% Tween-20 and 5 mM EDTA to stop the reaction. Samples were washed one time with 200 μL of the same PBS-Tween-EDTA buffer and resuspended in 100 μL of the buffer with added 0.1% BSA. Samples were measured using a CytoFlex LX flow cytometer (Beckman Coulter) equipped with six lasers, in either tube or plate sampling mode. Bleach and buffer wells were included between different samples to minimize potential sample carryover. Multiplex MOSAIC assays were carried out following the same protocol as for the single-plex MOSAIC assays, with different fluorescent dye-encoded beads combined in the same sample.

Plasma and saliva samples were diluted in Sample Diluent or StartingBlock™ Blocking Buffer (Thermo Fisher Scientific), respectively, with protease inhibitor (Halt™ Protease Inhibitor Cocktail, Thermo Fisher Scientific). Plasma samples were obtained from BioIVT and the Mass General Brigham Biobank, and saliva samples were obtained from BioIVT. All human samples were de-identified and experiments were performed under Institutional Review Board approval by Mass General Brigham. All plasma and saliva samples were centrifuged at 2000×g for 10 minutes or 21000×g for 20 minutes, respectively, at 4° C. before diluting for measurements.

TABLE 9
Assay conditions used for the MOSAIC and Simoa assays in this Example. The same detector antibody
concentrations and target capture times were used for each corresponding MOSAIC and Simoa assay.

| | MOSAIC | | | MOSAIC | | Simoa | |
|---|---|---|---|---|---|---|---|
| | Assay | | Detector | | Incubation | Streptavidin- | Incubation |
| | Bead | Assay | Antibody | Streptavidin- | Times | f3-galactosidase | Times |
| Analyte | Number | Type | (μg/mL) | DNA (pM) | (minutes) | (pM) | (minutes) |

Single-plex Assays

| | | | | | | | |
|---|---|---|---|---|---|---|---|
| IL-10 | 2,000-100,000 | 2-step | 0.3 | 300 | 60-15 | 150 | 60-5 |
| IFN-γ | | 2-step | 0.3 | 300 | 60-15 | 50 | 60-5 |
| IL-6 | | 3-step | 0.3 | 150 | 60-10-10 | 150 | 60-10-10 |
| IL-1f3 | | 2-step | 0.6 | 300 | 60-15 | 150 | 60-5 |
| IL-8 | | 3-step | 0.1 | 150 | 60-10-10 | 150 | 60-10-10 |
| IL-12p70 | | 2-step | 0.3 | 300 | 60-15 | 50 | 60-5 |

Four-plex Assay

| | | | | | | | |
|---|---|---|---|---|---|---|---|
| IL-6 | 50,000 | 3-step | 0.2 | 150 pM | 60-10-10 | 50 pM | 60-10-5 |
| IL-1f3 | 50,000 | | 0.6 | | | | |
| IL-10 | 20,000 | | 0.2 | | | | |
| IFN-γ | 20,000 | | 0.3 | | | | |

Eight-plex Assay (plasma/saliva)

| | | | | | | | |
|---|---|---|---|---|---|---|---|
| IFN-γ | 10,000/20,000 | 3-step | 0.3/0.3 | 150 pM | 60-10-10 | 50 pM | 60-10-5 |
| IL-1f3 | 20,000/50,000 | | 0.6/0.06 | | | (run as two | |
| IL-5 | 10,000/20,000 | | 0.6/0.6 | | | four-plex | |
| IL-6 | 20,000/20,000 | | 0.15/0.15 | | | assays) | |
| IL-10 | 10,000/20,000 | | 0.2/0.2 | | | | |
| IL-12p70 | 20,000/20,000 | | 0.3/0.3 | | | | |
| IL-18 | 10,000/20,000 | | 0.025/0.025 | | | | |
| VEGF | 20,000/20,000 | | 0.15/0.05 | | | | |

### Simoa Assays.

All Simoa assays were performed on an HD-X Analyzer (Quanterix), with automated sample processing, image analysis, and calculations of average enzyme per bead (AEB). The same antibody-coated beads used in the single-plex MOSAIC assays were used for all Simoa assays, with the same detector antibody concentrations as in the MOSAIC assays. 100,000 antibody-coated beads and 400,000 helper (non-conjugated) beads were used for each single-plex Simoa assay, while 125,000 antibody-coated beads per analyte were used for each four-plex Simoa assay. Streptavidin-β-galactosidase (SpG) Concentrate (Quanterix) was diluted in SpG Diluent (Quanterix) to the desired concentration for each assay. All assay reagents and consumables were loaded to the HD-X Analyzer according to the manufacturer's instructions.

### Data Analysis.

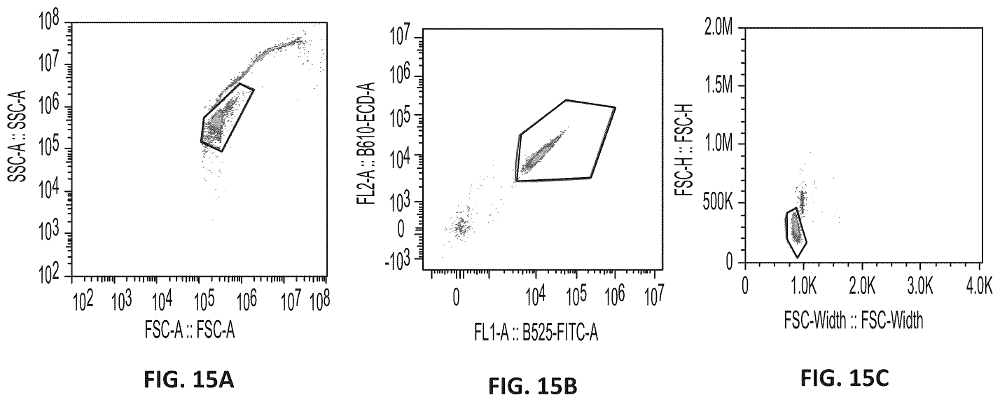

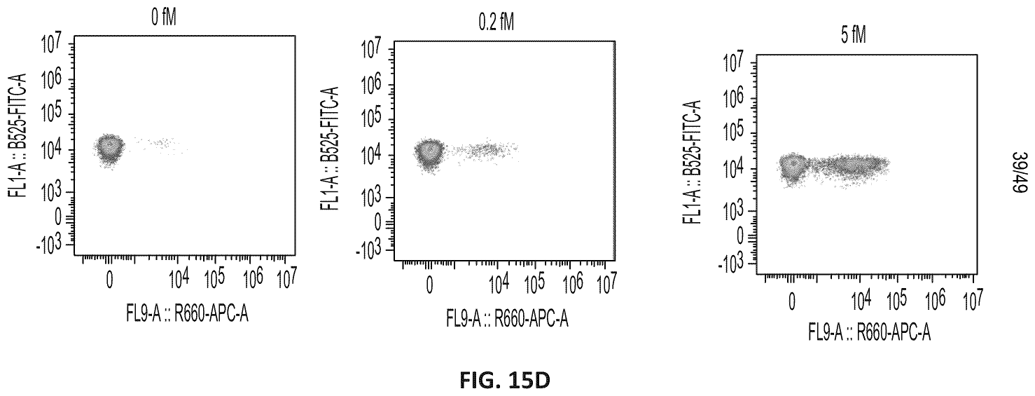

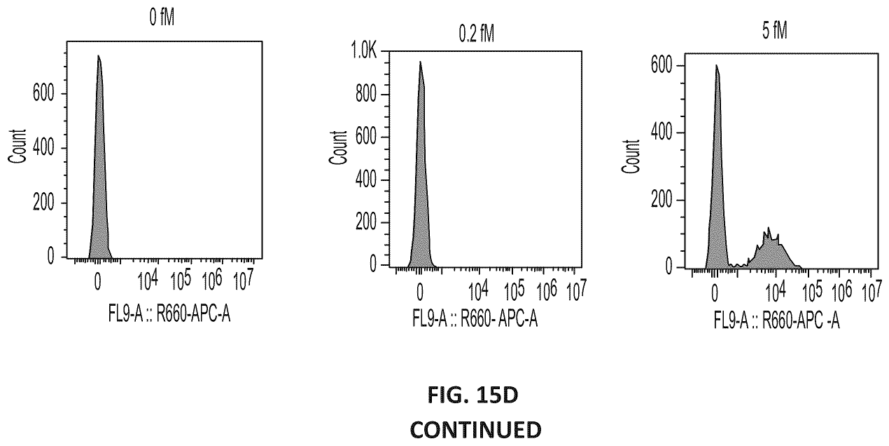

Flow cytometry data were first analyzed with FlowJo™ Software (Becton, Dickinson and Company); beads were identified using gates on forward scatter, side scatter, and bead fluorescence. Single beads were additionally gated using forward scatter (FIGS. 15A-D). The probe fluorescence intensities for each bead population were analyzed in Python with the packages jax, numpy, pandas, scikit-learn, and waltlabtools. A two-Gaussian mixture model was fit to the log-transformed fluorescence intensities for each well using expectation maximization. Two methods of counting “on” beads were calculated. In the first method, beads were assigned as “on” or “off” based on their predicted membership in one or the other Gaussian according to the expectation maximization algorithm. In the second method, beads were counted as “on” if they were at least five standard deviations above the mean of the lower (“off”) Gaussian peak. The first method yielded better results as long as two peaks were identifiable, but at very low numbers of “on” beads, the higher Gaussian was harder to fit, so the second method provided more reliable estimates. Accordingly, the metric used was a weighted average of the two, with a sinusoidal weighting function: the weight of the first method is given by sin⁴(πf/2), where f is the fraction of “on” beads according to the first method (Gaussian mixture assignment). The fraction of “on” beads was then converted to an average number of analyte molecules bound per bead using Poisson statistics, and mapped to concentration using a four-parameter logistic calibration curve. The LOD and LLOQ were calculated as three and ten standard deviations above the background, respectively, where a correction factor c₄(n) was applied for unbiased estimation of the standard deviation. Pearson correlation coefficients were calculated using GraphPad Prism.

More detailed version of the methods can be found in the supplementary method section below.

### Supplementary Methods

### Preparation of Antibody-Coated Capture Beads

1. Vortex the beads for 30 seconds and place them on the shaker.
2. If the antibody is lyophilized, reconstitute to 0.2 mg/mL in MES buffer.
3. If the antibody is in solution, buffer exchange.
   i. Measure the antibody stock concentration or mass using the spectrophotometer or the manufacturer's certificate of analysis. Calculate the volume of antibody stock solution containing 80 mg of antibody.
   ii. Add this volume, with enough MES buffer to bring the total volume to 500 μL, to a clean 50K Amicon filter in an Amicon microcentrifuge tube. Centrifuge at 14,000×g for 5 minutes.
   iii. Discard flow-through and quickly add 450 μL of MES buffer to the Amicon filter. Centrifuge at 14,000×g for 5 minutes. Repeat once more for a total of three 5-minute centrifugations.
   iv. Discard flow-through. Quickly invert a clean Amicon tube onto the top of the Amicon filter. Centrifuge the tube with the inverted Amicon filter at 1,000×g for 2 minutes.
   v. Rinse the filter membrane with 50 μL of MES buffer. Mix repeatedly. Centrifuge the tube with the inverted Amicon filter at 1,000×g for 2 minutes.
   vi. Measure the antibody concentration using the spectrophotometer, blanking with MES buffer. Pipette measure the volume of purified antibody. Dilute the antibody with MES buffer to bring it to 0.2 mg/mL.
4. Wash the beads.
   i. Determine the bead count using the particle size analyzer or the manufacturer's certificate of analysis. Calculate the volume of bead stock solution containing 4.2×10\hat{ }8 beads.
   ii. Transfer the volume of beads given in the table above into a test tube. Place the test tube on the magnetic separator.
   iii. Aspirate, remove from magnetic separator, add 300 μL of Bead Wash Buffer, vortex, pulse spin, and return to magnetic separator. Repeat two additional times with Bead Wash Buffer and two times with MES buffer.
   iv. Aspirate, remove from magnetic separator, and add 300 μL of MES buffer a third time, vortex and pulse spin, and leave beads in suspension.
5. Activate the beads.
   i. Carefully open one vial of EDC and slowly add 100 μL of MES buffer. Replace cap immediately and vortex briefly until dissolved.
   ii. Add 9 μL of reconstituted EDC to the tube of beads, vortex, and shake for 30 minutes at 4° C.
6. Conjugate the antibody to the beads.
   i. Pulse spin, place beads on magnetic separator, aspirate, remove from magnet, add 300 μL of MES buffer, vortex, pulse spin, and return to magnetic separator.
   ii. Aspirate, remove from magnetic separator, add the achieved batch volume of the buffer-exchanged antibody, and vortex. Shake for 2 hours at 4° C.
7. Block the beads.
   i. Pulse spin and place the beads on magnetic separator.
   ii. Aspirate, remove from magnetic separator, add 300 μL of Bead Wash Buffer, vortex, pulse spin, and return to magnetic separator. Repeat once.
   iii. Aspirate, remove from magnetic separator, and add 300 μL of Bead Blocking Buffer, and vortex. Shake for 45 minutes at room temperature.
8. Perform the final wash and resuspension.
   i. Pulse spin and place the beads on magnetic separator.
   ii. Aspirate, remove from magnetic separator, add 300 μL of Bead Wash Buffer, vortex, pulse spin, and return to magnetic separator. Repeat once.
   iii. Aspirate, remove from magnetic separator, add 300 μL of Bead Diluent, vortex, and pulse spin.
   iv. Determine the final bead count using the particle size analyzer.
      Note: Bead coupling can alternatively be performed at room temperature, in which 2.8×10′ starting beads are used, and EDC activation and antibody conjugation steps are performed at room temperature.

### Detection Antibody Biotinylation

1. If the antibody is lyophilized, reconstitute to 1 mg/mL in PBS.
2. If the antibody is in solution, buffer exchange.
   i. Measure the antibody stock concentration or mass using the spectrophotometer or the manufacturer's certificate of analysis. Calculate the volume of antibody stock solution containing 130 mg of antibody.
   ii. Add this volume, with enough PBS to bring the total volume to 500 μL, to a clean 50K Amicon filter in an Amicon microcentrifuge tube. Centrifuge at 14,000×g for 5 minutes.
   iii. Discard flow-through and quickly add 450 μL of PBS to the Amicon filter. Centrifuge at 14,000×g for 5 minutes. Repeat once more for a total of three 5-minute centrifugations.
   iv. Discard flow-through. Quickly invert a clean Amicon tube onto the top of the Amicon filter. Centrifuge the tube with the inverted Amicon filter at 1,000×g for 2 minutes.
   v. Rinse the filter membrane with 50 μL of PBS. Mix repeatedly. Centrifuge the tube with the inverted Amicon filter at 1,000×g for 2 minutes.
   vi. Measure the antibody concentration using the spectrophotometer, blanking with PBS. Pipette measure the volume of purified antibody. Dilute the antibody with PBS to bring it to 1 mg/mL.
3. Biotinylate the detection antibody.
   i. Reconstitute the biotin to an 8.9 mM stock concentration by adding 383 μL of deionized water.
   ii. Add 100 μL of antibody and 3 μL of biotin to a clean test tube, vortex, pulse spin, and incubate for 30 minutes at room temperature.
4. Purify the biotinylated antibody.
   i. Transfer the contents of the biotinylation reaction tube to a clean Amicon filter in an Amicon microcentrifuge tube. Wash the biotinylation reaction tube with 400 μL of PBS and transfer it to the Amicon filter as well. Centrifuge at 14,000×g for 5 minutes.
   ii. Discard flow-through and quickly add 450 μL of PBS to the Amicon filter. Centrifuge at 14,000×g for 5 minutes. Repeat three more times for a total of five 5-minute centrifugations.
   iii. Discard flow-through. Quickly invert a clean Amicon tube onto the top of the Amicon filter. Centrifuge the tube with the inverted Amicon filter at 1,000×g for 2 minutes.
   iv. Rinse the filter membrane with 50 μL of PBS. Mix repeatedly. Centrifuge the tube with the inverted Amicon filter at 1,000×g for 2 minutes.
   v. Measure the antibody concentration using the spectrophotometer, blanking with PBS.

### Preparation of Streptavidin-DNA Conjugate

1. Make a solution of 35.8 μM primer and 43.0 μM template in 1× Quick Ligation Buffer.
2. Heat the solution at 95° C. for two minutes and allow to cool to room temperature over 90 minutes.
3. Add T4 DNA ligase (6 μL of 2,000,000 U/mL per 100 μL reaction mixture) and incubate at room temperature for three hours.
4. Heat at 65° C. for 10 minutes to inactivate the ligase, and cool to room temperature.
5. Buffer exchange into PBS with 1 mM EDTA using a Zeba spin desalting column.
6. Measure concentration with the spectrophotometer, using the molecular weights of the primer and template and the initial primer concentration in the ligation reaction to determine the buffer exchanged primer concentration.
7. Buffer exchange streptavidin into PBS using a 10K Amicon filter and measure the concentration with the spectrophotometer.
8. Incubated streptavidin with a 20-fold molar excess of DBCO-PEG4-NHS, dissolved to 5 mg/mL in dimethyl sulfoxide, for 30 minutes at room temperature.
9. Purify DBCO-modified streptavidin with a 10K Amicon centrifugal filter in PBS with 1 mM EDTA, with four centrifuge cycles of 14,000×g for five minutes and a final centrifuge cycle of 14,000×g for ten minutes. Measure the concentration with the spectrophotometer.
10. Add a two-fold molar excess of the ligated primer-template (molar excess calculated relative to the primer concentration, determined in step 5) to the DBCO-modified streptavidin and incubate overnight at 4° C.
11. Store the conjugate in aliquots at −80° C. in PBS with 5 mM EDTA, 0.1% BSA, and 0.02% sodium azide.

### MOSAIC Assay Procedure

For all washes on the plate washer, use System Wash Buffer 1 and ensure that beads are held at the bottom of the plate by the magnetic attachment. Each wash cycle is 170 μL.
1. Vortex beads for 30 seconds and dilute in Bead Diluent to the appropriate concentration to yield the desired number of beads in 10 μL/sample. We recommend starting with 20,000 beads/sample, which is 2×10≢beads/mL. For multiplexing, mix the corresponding amount of each bead type.
2. Perform serial dilutions of protein standard in sample diluent for the calibration curve.
3. Centrifuge samples and dilute in sample diluent. We recommend:
   a. Plasma/serum: centrifuge at 2,000×g for 10 minutes and dilute 4-16 fold.
   b. Saliva: centrifuge at 21,000×g for 20 minutes and dilute 4-16 fold.
   c. Urine: centrifuge at 10,000×g for 5 minutes and dilute 1-2 fold.
4. Add 100 μL of diluted sample (or calibrator) and 10 μL of beads to each well.
5. If performing a two-step assay, dilute the biotinylated detection antibody to 3.6 μg/mL. Add 10 μL of the detection antibody to each well.
6. Seal the plate and incubate for one hour at room temperature, shaking at 650 rpm.
7. If performing a three-step assay:
   i. Wash the samples three times on the plate washer.
   ii. Dilute the biotinylated detection antibody to 0.3 μg/mL.
   iii. Put the plate on the plate magnet to draw beads to one side of the well. Aspirate and add 100 μL of the detection antibody to each well.
   iv. Seal the plate and incubate for 10 minutes at room temperature, shaking at 600 rpm.
8. Wash the samples six times on the plate washer.
9. Put the plate on the plate magnet to draw beads to one side of the well. Aspirate and add 100 μL of streptavidin-DNA conjugate, diluted to 300 μM (for a two-step assay) or 150 μM (for a three-step assay) in sample diluent with 5 mM EDTA, to each well.
10. Seal the plate and incubate for 15 minutes (for a two-step assay) or 10 minutes (for a three-step assay) at room temperature, shaking at 650 rpm.
11. Buffer exchange the phi29 polymerase with a Zeba desalting column into 10 mM Tris-HCl (pH 7.5), 100 mM KCl, 0.1 mM EDTA, 0.5% Tween-20, and 0.5% NP-40 to remove dithiothreitol.
12. Make the RCA reaction mixture:
   0.5 mM dNTP mix
   0.33 U/uL phi29 DNA polymerase
   0.2 mg/mL BSA
   1 nM fluorescently-labeled DNA probe
   0.1% Tween-20
   50 mM Tris-HCl
   10 mM ammonium sulfate
   10 mM magnesium chloride
13. Wash the samples eight times on the plate washer.
14. Pipette mix to resuspend beads and transfer samples to a new 96-well plate.
15. Put the plate on the plate magnet to draw beads to one side of the well. Aspirate and add 200 μL of System Wash Buffer. Aspirate, and resuspend in 60 μL of the RCA reaction mixture.
16. Seal the plate and incubate for one hour at 37° C., shaking at 650 rpm.
17. Add 150 μL PBS with 0.1% Tween-20 and 5 mM EDTA to each well to stop the reaction. Put the plate on the plate magnet to draw beads to one side of the well, and aspirate (do not aspirate completely dry). Add 200 μL of the same PBS-Tween-EDTA buffer, aspirate, and resuspend in 100 μL of the buffer with added 0.1% BSA.
18. Measure samples on the flow cytometer within 24 hours. Run bleach and either buffer or water between different samples to minimize potential sample carryover.
19. Identify bead populations using flow cytometry analysis software. We recommend gating beads based on size/shape (forward and side scatter), followed by fluorescence.
20. Separate on-beads from off-beads using gates in flow cytometry analysis software or the waltlabtools.mosaic Python module.
21. Using software such as the waltlabtools Python package:
   i. Convert the fraction of on-beads for each sample to the average number of molecules per bead (AMB): AMB=−log (1−fₒₙ).
   ii. Regress a 4-parameter logistic calibration curve to the calibrator concentrations and AMBs.
   iii. Use the calibration curve to calculate the concentrations of the samples.
      Incubation times; sample dilutions and diluents; assay protocol (two-step vs. three-step); and concentrations of beads, detection antibody, and streptavidin-DNA conjugate can be optimized for each assay or application.

### REFERENCES

1 Rissin, D. M. et al. Single-molecule enzyme-linked immunosorbent assay detects serum proteins at subfemtomolar concentrations. Nature Biotechnology 28, 595-599, doi:10.1038/nbt.1641 (2010).
2 Cohen, L. & Walt, D. R. Highly Sensitive and Multiplexed Protein Measurements. Chemical Reviews 119, 293-321, doi:10.1021/acs.chemrev.8b00257 (2019).
3 Yelleswarapu, V. et al. Mobile platform for rapid sub-picogram-per-milliliter, multiplexed, digital droplet detection of proteins. Proceedings of the National Academy of Sciences 116, 4489, doi:10.1073/pnas.1814110116 (2019).
4 Cohen, L. et al. Single Molecule Protein Detection with Attomolar Sensitivity Using Droplet Digital Enzyme-Linked Immunosorbent Assay. ACS Nano 14, 9491-9501, doi:10.1021/acsnano.0c02378 (2020).
Chang, L. et al. Single molecule enzyme-linked immunosorbent assays: Theoretical considerations. Journal of Immunological Methods 378, 102-115, doi:https://doi.org/10.1016/j.jim.2012.02.011 (2012).
6 Wu, C., Garden, P. M. & Walt, D. R. Ultrasensitive Detection of Attomolar Protein Concentrations by Dropcast Single Molecule Assays. Journal of the American Chemical Society 142, 12314-12323, doi:10.1021/jacs.0c04331 (2020).
7 Maley, A. M., Garden, P. M. & Walt, D. R. Simplified Digital Enzyme-Linked Immunosorbent Assay Using Tyramide Signal Amplification and Fibrin Hydrogels. ACS Sensors 5, 3037-3042, doi:10.1021/acssensors.Oc01661 (2020).
8 Kan, C. W. et al. Digital enzyme-linked immunosorbent assays with sub-attomolar detection limits based on low numbers of capture beads combined with high efficiency bead analysis. Lab on a Chip 20, 2122-2135, doi:10.1039/DOLC00267D (2020).
9 Smith, L. D. et al. High-Fidelity Single Molecule Quantification in a Flow Cytometer Using Multiparametric Optical Analysis. ACS Nano 14, 2324-2335, doi:10.1021/acsnano.9b09498 (2020).
10 Akama, K., Shirai, K. & Suzuki, S. Droplet-Free Digital Enzyme-Linked Immunosorbent Assay Based on a Tyramide Signal Amplification System. Analytical Chemistry 88, 7123-7129, doi:10.1021/acs.analchem.6b01148 (2016).
11 Yang, K. S. et al. Extracellular Vesicle Analysis Allows for Identification of Invasive IPMN. Gastroenterology 160, 1345-1358.e1311, doi:10.1053/j.gastro.2020.11.046 (2021).
12 Iyer, S. S. & Cheng, G. Role of interleukin 10 transcriptional regulation in inflammation and autoimmune disease. Crit Rev Immunol 32, 23-63, doi:10.1615/critrevimmunol.v32.i1.30 (2012).
13 Moore, K. W., de Waal Malefyt, R., Coffman, R. L. & O'Garra, A. Interleukin-10 and the Interleukin-10 Receptor. Annual Review of Immunology 19, 683-765, doi:10.1146/annurev.immunol.19.1.683 (2001).
14 Reynolds, D. in Encyclopedia of Biometrics (eds Stan Z. Li & Anil Jain) 659-663 (Springer US, 2009).
15 Rivnak, A. J. et al. A fully-automated, six-plex single molecule immunoassay for measuring cytokines in blood. Journal of Immunological Methods 424, 20-27, doi:https://doi.org/10.1016/j.jim.2015.04.017 (2015).
16 Ahmad, R. et al. A rapid triage test for active pulmonary tuberculosis in adult patients with persistent cough. Science Translational Medicine 11, eaaw8287, doi:10.1126/scitranslmed.aaw8287 (2019).
17 Norman, M. et al. Ultra-Sensitive High-Resolution Profiling of Anti-SARS-CoV-2 Antibodies 50 for Detecting Early Seroconversion in COVID-19 Patients. medRxiv, 2020.2004.2028.20083691, doi:10.1101/2020.04.28.20083691 (2020).
18 Tobos, C. I., Sheehan, A. J., Duffy, D. C. & Rissin, D. M. Customizable Multiplex Antibody Array Immunoassays with Attomolar Sensitivities. Analytical Chemistry 92, 5613-5619, doi:10.1021/acs.analchem.0c00631 (2020).
19 Gilboa, T., Maley, A. M., Ogata, A. F., Wu, C. & Walt, D. R. Sequential Protein Capture in Multiplex Single Molecule Arrays: A Strategy for Eliminating Assay Cross-Reactivity. Advanced Healthcare Materials n/a, 2001111, doi:10.1002/adhm.202001111 (2020).

### Example 2. Barcoded Multiplex MOSAIC Assay

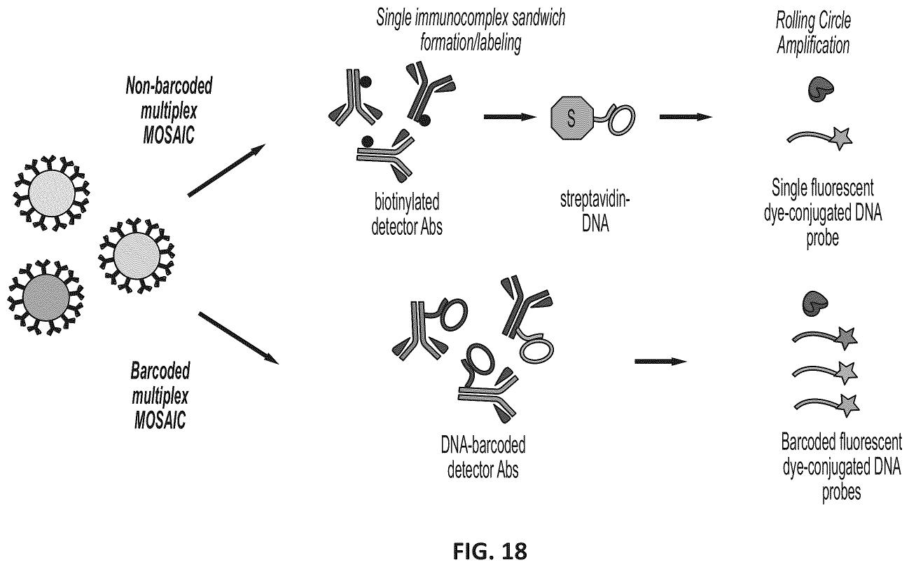

This example describes the barcoded multiplex MOSAIC assay. In barcoded multiplex MOSAIC assays, a unique pair of fluorescent dye-encoded capture bead and DNA template-barcoded detector antibody was used for each analyte (FIG. 18). The capture beads were prepared as described herein, and the antibody-DNA conjugates were prepared as detailed below. Each unique DNA template-conjugated antibody, when amplified during the RCA reaction, was labeled with a specific fluorescent dye-conjugated DNA probe or ratiometric combination of dye-conjugated DNA probes. Each probe color or ratiometric combination of colors can be distinguished using multiple detection channels in flow cytometry. Upon capture of single analyte molecules on beads, a mixture of detector antibodies conjugated with unique primer-template sequences was added, followed by washing and resuspension in an RCA reaction that contained a mixture of fluorescent dye-conjugated DNA probes. Each analyte corresponded to a unique pair of (1) capture bead color, fluorescence intensity, or size; and (2) fluorescent probe color or ratio of colors. As a result, only the “correct” matched pairs of capture bead and probe signal were classified as “on” beads for each analyte, with “wrong” pairs of capture bead and probe colors, i.e., cross-reactive binding events, being eliminated from the analysis. In contrast, in the non-barcoded multiplex MOSAIC format, all detector antibodies were biotinylated and labeled with the same streptavidin-DNA conjugate and fluorescent probe.

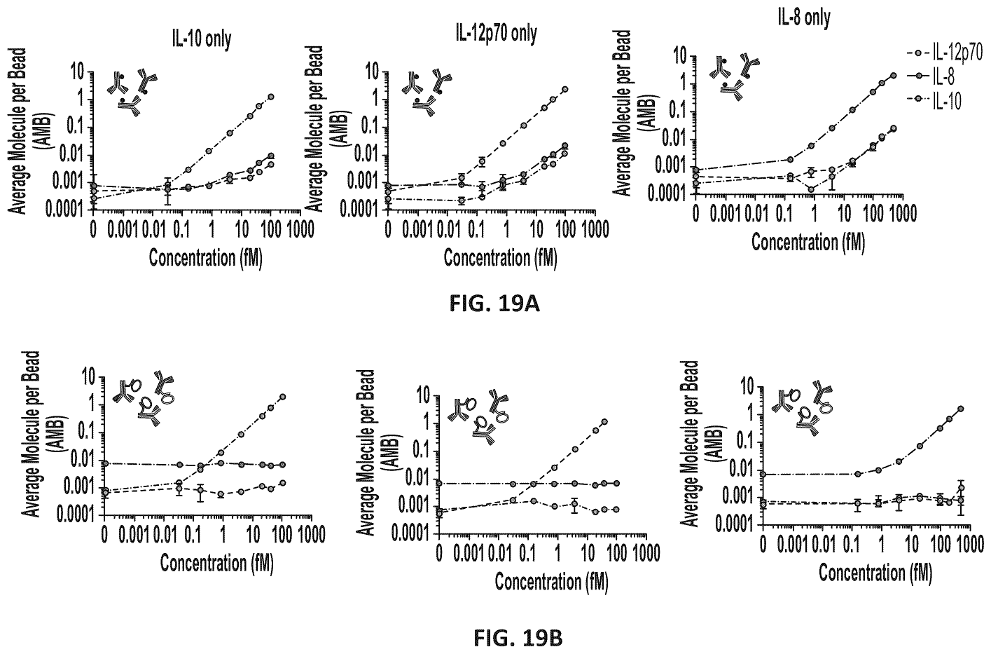

FIG. 19A and FIG. 19B depict the dropout curves with increasing concentrations of each individual target protein for non-barcoded multiplex MOSAIC (FIG. 19A) and barcoded multiplex MOSAIC (FIG. 19B). As shown by the figures, the inclusion of only the correct capture bead-probe color pairs in barcoded multiplex MOSAIC enables elimination of cross-reactive signals and, thus, providing more accurate multiplex measurements.

### Preparation of Antibody-DNA Conjugates.

For each detector antibody, a 5′ azide-modified primer was annealed to a unique DNA template by heating a solution of 30 μM primer and 30.3 μM template in NEBNext Quick Ligation Buffer (New England Biolabs) at 95° C. for two minutes and allowing to cool to room temperature over 90 minutes. Ligation was then performed with addition of T4 DNA ligase and incubation at room temperature for two hours. The ligation reaction was buffer exchanged into phosphate buffered saline (PBS) with 1 mM EDTA using a 7K MWCO Zeba spin desalting column (Thermo Fisher Scientific). For conjugation, the detector antibody was either reconstituted into PBS from lyophilized form or buffer exchanged into PBS using a 50K Amicon Ultra-0.5 mL centrifugal filter, incubated with a 20-fold molar excess of dibenzocyclooctyne-PEG4-N-hydroxysuccinimidyl ester (DBCO-PEG4-NHS, MilliporeSigma) for 30 minutes at room temperature, and purified with a 50K Amicon Ultra-0.5 mL centrifugal filter in PBS with 1 mM EDTA. A two-fold molar excess of the ligated primer-template was then added to the DBCO-modified antibody and incubated overnight at 4° C. The conjugate was stored in aliquots at −80° C. in PBS with 5 mM EDTA, 0.1% BSA, and 0.02% sodium azide.

What is claimed is:

1. A method of detecting a target analyte in a sample, the method comprising:
(a) contacting a sample containing or suspected of containing the target analyte with a plurality of beads comprising a capture moiety that specifically binds to the target analyte, under conditions and for a time sufficient for the target analyte in the sample to bind to the capture moiety,
wherein a plurality of the beads are associated with zero target analyte molecules;
wherein a plurality of the beads are associated with one target analyte molecule;
wherein at least about 20% of the beads are associated with either zero or one target analyte molecule;
(b) contacting the product of step (a) with a detecting moiety that binds to the target analyte,
(c) contacting the product of step (b) with a signal amplification moiety that binds to the detecting moiety to generate a detectable signal for each bead carrying the target analyte; and
(d) detecting the detectable signal by flow cytometry, thereby detecting the target analyte in the sample.

2. The method of claim 1,
(a) wherein the bead comprises a magnetic bead, a paramagnetic bead, a non-magnetic bead, a porous bead, or a glass bead;
(b) wherein the capture moiety comprises an antibody, an aptamer, an antibody mimetic, a polypeptide, a nucleic acid, a molecularly-imprinted polymer, a receptor, or a small molecule;
(c) wherein the detecting moiety comprises an antibody, an aptamer, an antibody mimetic, a polypeptide, a nucleic acid, a molecularly-imprinted polymer, a receptor, a binding protein, or a small molecule;
(d) wherein the signal amplification moiety comprises an enzyme and/or a nucleic acid molecule;
(e) wherein the detectable signal is generated by rolling circle amplification followed by hybridization with a complementary fluorescently labeled DNA probe; rolling circle transcription; hybridization chain reaction; loop-mediated isothermal amplification; radical polymerization; tyramide signal amplification (TSA); enzyme-catalyzed proximity labeling (PL) polymerization; labeling with a pre-amplified signal with fluorescently labeled enzymes, nanoparticles or nucleic acid concatemers; polymerization-based signal amplification; or magnetic bead-quantum dot immunoassays;
(f) wherein at least about 30%, 40%, 50%, 60%, 70%, 80%, 90%, 95%, 98%, or 99% of the beads are associated with either zero or one target analyte molecule; and/or
(g) wherein the beads comprising the capture moiety for the target analyte are different from beads comprising a capture moiety for a non-target analyte, and/or with different colors, shapes, or sizes.

3. The method of claim 1,
(a) wherein the detecting moiety and the signal amplification moiety are linked directly; or
(b) wherein the detecting moiety and the signal amplification moiety are linked by a non-covalent affinity binding pair, wherein the detecting moiety is linked to a first member of the non-covalent affinity binding pair, and the signal amplification moiety is linked to a second member of the non-covalent affinity binding pair.

4. The method of claim 3, wherein the non-covalent affinity binding pair is biotin-streptavidin, biotin-avidin, ligand-receptor, antigen-antibody, or antibody binding protein-antibody.

5. The method of claim 1, wherein the method reduces cross-reactivity or non-specific binding.

6. The method of claim 5, wherein the cross-reactivity or non-specific binding is reduced by detecting the beads and the detectable signal by flow cytometry.

7. The method of claim 1,
(a) wherein the target analyte is a protein, a nucleic acid, a polysaccharide, a lipid, a cell, a fatty acid, a therapeutic agent, an organism, a virus, a toxin, a peptide, an oligosaccharide, a lipoprotein, a glycoprotein, a glycan, or a hormone;
(b) wherein the sample comprises a biological sample;
wherein the biological sample is
(i) a body fluid selected from the group consisting of lymph, whole blood, plasma, serum, a blood fraction containing peripheral blood mononuclear cells, urine, saliva, semen, sweat, lacrimal fluid, synovial fluid, cerebrospinal fluid, feces, mucous, vaginal fluid, and spinal fluid, or
(ii) a breast tissue, a liver tissue, a pancreatic tissue, a cervix tissue, a lung tissue, a renal tissue, a colonic tissue, a brain tissue, a muscle tissue, a synovial tissue, skin, a hair follicle, bone marrow, a tumor tissue, a tissue lysate or homogenate, or an organ lysate or homogenate; or
(iii) plasma, or
(iv) saliva.

8. The method of claim 1, wherein steps (a), (b), (c), or any combination thereof, are performed sequentially or simultaneously.

9. The method of claim 1,
(a) further comprising detecting the beads comprising the capture moiety by flow cytometry; and/or
(b) further comprising measuring a concentration of the target analyte in the sample, wherein the concentration of the target analyte in the sample is proportional to the level of the detectable signal.

10. The method of claim 1,
(a) further comprising detecting or measuring a concentration of an additional target analyte in the sample;
(i) wherein the additional target analyte comprises one, two, three, four, five, six, seven, eight, nine, ten or more target analytes; and/or
(ii) wherein the additional target analyte is a protein, a nucleic acid, a polysaccharide, a lipid, a cell, a fatty acid, a therapeutic agent, an organism, a virus, a toxin, a peptide, an oligosaccharide, a lipoprotein, a glycoprotein, a glycan, or a hormone; and/or
(b) further comprising contacting the sample with (i) a plurality of beads comprising an additional capture moiety that specifically binds to the additional target analyte; (ii) an additional detecting moiety that binds to the additional target analyte, and (iii) an additional signal amplification moiety that binds to the additional detecting moiety to generate an additional detectable signal.

11. The method of claim 1,
(a) wherein the method has a limit of detection of about 0.1 aM to about 1 mM;
(b) wherein the limit of detection is about 0.1 aM to about 1 mM, about 0.1 aM to about 1 μM, about 0.1 aM to about 1 nM, about 0.1 aM to about 1 μM, about 0.1 aM to about 1 fM, about 0.1 aM to about 900 aM, about 0.1 aM to about 800 aM, about 0.1 aM to about 700 aM, about 0.1 aM to about 600 aM, about 0.1 aM to about 500 aM, about 0.1 aM to about 400 aM, about 0.1 aM to about 300 aM, about 0.1 aM to about 200 aM, or about 0.1 aM to about 100 aM; and/or
(c) wherein the limit of detection is about 1 fM, about 900 aM, about 800 aM, about 700 aM, about 600 aM, about 500 aM, about 400 aM, about 300 aM, about 200 aM, about 100 aM, about 90 aM, about 80 aM, about 70 aM, about 60 aM, about 50 aM, about 40 aM, about 30 aM, about 20 aM, about 10 aM, or about 1 aM, or about 0.1 aM.

12. The method of claim 1, wherein the signal detection takes less than about one minute per sample, less than about 45 seconds per sample, or less than about 30 seconds per sample.

13. The method of claim 1, (a) wherein the sample is contacted with about 2,000 to about 100,000 beads; and/or
(b) wherein the sample is contacted with about 2,000 beads, about 5,000 beads, about 10,000 beads, about 20,000 beads, about 50,000 beads, or about 100,000 beads.

14. The method of claim 1, (a) wherein the beads and the sample are incubated for about 1 min to about 48 h, about 1 min to about 10 h, or about 1 h to about 4 h; and/or
(b) wherein the beads and the sample are incubated for about 10 minutes, about 20 minutes, about 30 minutes, about 40 minutes, about 50 minutes, about 1 h, about 2 h, about 3 h, about 4 h, or about 5 h.

15. A method of detecting a first target analyte and a second target analyte in a sample, the method comprising:
(a) contacting a sample containing or suspected of containing the first target analyte and/or the second target analyte with (i) a plurality of first beads comprising a first capture moiety that specifically binds to the first target analyte, and (ii) a plurality of second beads comprising a second capture moiety that specifically binds to the second target analyte, under conditions and for a time sufficient for the first target analyte in the sample to bind to the first capture moiety and for the second target analyte in the sample to bind to the second capture moiety,
wherein a plurality of the first beads are associated with zero first target analyte molecules; wherein a plurality of the first beads are associated with one first target analyte molecule; wherein at least about 20% of the first beads are associated with either zero or one first target analyte molecule; and
wherein a plurality of the second beads are associated with zero second target analyte molecules; wherein a plurality of the second beads are associated with one second target analyte molecule; wherein at least about 20% of the second beads are associated with either zero or one second target analyte molecule;
(b) contacting the product of step (a) with (i) a first detecting moiety that binds to the first target analyte, and (ii) a second detecting moiety that binds to the second target analyte;
(c) contacting the product of step (b) with (i) a first signal amplification moiety that binds to the first detecting moiety to generate a first detectable signal for each bead carrying the first target analyte, and (ii) a second signal amplification moiety that binds to the second detecting moiety to generate a second detectable signal for each bead carrying the second target analyte; and
(d) detecting the first detectable signal and the second detectable signal by flow cytometry, thereby detecting the first target analyte and the second target analyte in the sample.

16. The method of claim 15,
(a) wherein the first detectable signal and the second detectable signal are different signals, and/or with different colors;
(b) wherein the first beads comprising the first capture moiety and the second beads comprising the second capture moiety are different, and/or with different colors, shapes, or sizes;
(c) wherein the method further comprises detecting the first beads comprising the first capture moiety and the second beads comprising the second capture moiety by flow cytometry; and/or
(d) wherein the method reduces cross-reactivity or non-specific binding.

17. The method of claim 15,
(a) wherein the method has a limit of detection of about 0.1 aM to about 1 mM;
(b) wherein the limit of detection is about 0.1 aM to about 1 mM, about 0.1 aM to about 1 μM, about 0.1 aM to about 1 nM, about 0.1 aM to about 1 μM, about 0.1 aM to about 1 fM, about 0.1 aM to about 900 aM, about 0.1 aM to about 800 aM, about 0.1 aM to about 700 aM, about 0.1 aM to about 600 aM, about 0.1 aM to about 500 aM, about 0.1 aM to about 400 aM, about 0.1 aM to about 300 aM, about 0.1 aM to about 200 aM, or about 0.1 aM to about 100 aM; and/or
(c) wherein the limit of detection is about 1 fM, about 900 aM, about 800 aM, about 700 aM, about 600 aM, about 500 aM, about 400 aM, about 300 aM, about 200 aM, about 100 aM, about 90 aM, about 80 aM, about 70 aM, about 60 aM, about 50 aM, about 40 aM, about 30 aM, about 20 aM, about 10 aM, or about 1 aM, or about 0.1 aM.

18. The method of claim 15, wherein the signal detection takes less than about one minute per sample, less than about 45 seconds per sample, or less than about 30 seconds per sample.

19. The method of claim 15, (a) wherein the sample is contacted with about 2,000 to about 100,000 beads; and/or (b) wherein the sample is contacted with about 2,000 beads, about 5,000 beads, about 10,000 beads, about 20,000 beads, about 50,000 beads, or about 100,000 beads.

20. The method of claim 15, (a) wherein the beads and the sample are incubated for about 1 min to about 48 h, about 1 min to about 10 h, or about 1 h to about 4 h; and/or (b) wherein the beads and the sample are incubated for about 10 minutes, about 20 minutes, about 30 minutes, about 40 minutes, about 50 minutes, about 1 h, about 2 h, about 3 h, about 4 h, or about 5 h.
